(19) United States
(12) Patent Application Publication
(10) Pub. No.: US 2026/0174024 A1
(43) Pub. Date: Jun. 25, 2026

(54) Brassica Plants Producing Elevated Levels of Polyunsaturated Fatty Acids

(71) Applicant: BASF PLANT SCIENCE COMPANY GMBH, Ludwigshafen (DE)

(72) Inventors: Richard Fletcher, Windsor, CO (US); Kristin P. Monser-Gray, Fort Collins, CO (US)

(21) Appl. No.: 19/420,423

(22) Filed: Dec. 15, 2025

Related U.S. Application Data
(63) Continuation of application No. 17/430,937, now abandoned.
(60) Provisional application No. 62/805,743, filed on Feb. 14, 2019.
(60) Provisional application No. 62/896,343, filed on Sep. 5, 2019.

(51) Int. Cl. A01H 1/00; A01H 1/04; C07K 14/415; C12N 15/82

(52) U.S. Cl. CPC A01H 1/104; A01H 1/045; C07K 14/415; C12N 15/8247

(57) ABSTRACT

Provided herein are Brassica plants that produce one or more of omega-3 docosapentaenoic acid (DHA), docosapentaenoic acid (DPA), and eicosapentaenoic acid (EPA), and methods of making such plants.

### CROSS-REFERENCE TO RELATED APPLICATIONS

This application is a continuation of U.S. application Ser. No. 17/430,937, which is the U.S. National Stage of International Application No. PCT/US2020/018413, filed Feb. 14, 2020, which claims the benefit U.S. Provisional Application Nos. 62/805,743 and 62/896,343, filed on Feb. 14, 2019 and Sep. 5, 2019, respectively; all of the aforementioned applications are hereby incorporated by reference herein in their entireties.

### SEQUENCE LISTING

The Sequence Listing, which is a part of the present disclosure, is submitted concurrently with the specification as an XML file. The name of the XML file containing the Sequence Listing is “230223A_SubSeqListing.xml”, which was created on Jan. 14, 2026, and is 239,560 bytes in size. The subject matter of the Sequence Listing is incorporated by reference herein in its entirety.

### TECHNICAL FIELD

This disclosure describes production of omega-3 docosahexaenoic acid (DHA), docosapentaenoic acid (DPA), and/or eicosapentaenoic acid (EPA) at elevated levels in seeds of transgenic Brassica plants. Seeds and oils obtained from such seeds that have higher levels of DHA, DPA, and/or EPA have certain beneficial effects in their fatty acid profiles, such as reductions in the levels of saturated fatty acids (e.g., stearic acid).

### BACKGROUND

Aquaculture is a fast-growing industry where shrimp and various fish such as salmon, tilapia, halibut, carp, channel catfish, trout, sea bream and sea bass can be grown under controlled conditions. Typically, farmed fish are fed formulations containing fish oil and/or omega-3 long chain polyunsaturated fatty acids such as eicosapentaenoic acid (EPA) and docosahexaenoic acid (DHA) to ensure that the farmed fish can deliver the health benefits of the omega-3 fish oils to consumers. Aquaculture production is expected to grow several times in the coming decades, while fishmeal and fish oil production will be about constant. As such, there is a need for alternative sources of omega-3 long chain polyunsaturated fatty acids.

### SUMMARY

This disclosure is based, at least in part, on the discovery that Brassica plants can be produced in which the seeds from these plants yield oils with elevated levels of long chain polyunsaturated fatty acids such as omega-3 docosahexaenoic acid (DHA), docosapentaenoic acid (DPA), and/or eicosapentaenoic acid (EPA). For example, the Brassica plants, or parts thereof, described herein can have higher levels of EPA, higher levels of DPA, higher levels of DHA, higher levels of EPA and DHA, higher levels of DHA and DPA, higher levels of DPA and EPA, or higher levels of EPA, DHA, and DPA.

In one aspect, this disclosure provides a Brassica plant or a part thereof comprising one or more exogenous polynucleotides heritably integrated into its genome, the exogenous polynucleotides comprising one or more expression cassettes having nucleotide sequences encoding one or more d12DES, one or more d6Elo, one or more d6Des, one or more d5Des, one or more d5Elo, one or more d4Des, and/or one or more o3Des. The plant can be the result of crossing a first parental Brassica plant that comprises the one or more exogenous polynucleotides with a second parental Brassica plant. The Brassica plant produces in its seeds a greater amount of one or more polyunsaturated fatty acids selected from the group consisting of EPA, DPA, and DHA than the first parental Brassica plant and/or the second parental Brassica plant. A part of a Brassica plant includes any parts derived from a plant, including cells, tissues, roots, stems, leaves, non-living harvest material, silage, seeds, seed meals and pollen.

In another aspect, provided is a method of producing a Brassica plant or a part thereof, the method comprising crossing a first Brassica parent plant producing one or more polyunsaturated fatty acids selected from the group consisting of EPA, DPA, and DHA in its seeds with a second Brassica parent plant to produce progeny plants producing one or more of EPA, DPA, and DHA. In some embodiments, the progeny Brassica plant produces greater levels of DHA and/or EPA than the first or second Brassica parent. In some embodiments, the first Brassica plant has one or more exogenous polynucleotides (e.g., T-DNAs) heritably integrated into its genome. In some embodiments, the second Brassica plant contributes at least one genomic sequence that confers in part, or in whole, the higher amount of one or more of EPA, DPA, and DHA. This genomic sequence can be referred to as a quantitative trait locus (QTL). The genomic sequence from the second parent can be all or a part of the genomic sequence selected from the group consisting of a) the genomic sequence on chromosome N1 between nucleotide positions 8879780 and 11922690; b) the genomic sequence on chromosome N1 between nucleotide positions 22823086 and 24045492; and c) the genomic sequence on chromosome N6 between nucleotide positions 19156645 and 20846412.

This disclosure also provides a Brassica plant or a part thereof that includes (i) one or more exogenous polynucleotides (e.g., T-DNAs) heritably integrated into its genome, the exogenous polynucleotides comprising one or more expression cassettes having nucleotide sequences encoding one or more desaturases and/or one or more elongases; and (ii) all or part of at least one genomic sequence of a B. napus parent genome that confers a higher amount of one or more polyunsaturated fatty acids selected from the group consisting of EPA, DPA, and DHA, wherein the genome sequence is selected from the group consisting of: a) the genomic sequence on chromosome N1 between nucleotide positions 8,879,780 and 11,922,690; b) the genomic sequence on chromosome N1 between nucleotide positions 22,823,086 and 24,045,492; and c) the genomic sequence on chromosome N6 between nucleotide positions 19,156,645 and 20,846,412; wherein seeds of the Brassica plant have a greater amount of one or more polyunsaturated fatty acids selected from the group consisting of EPA, DPA, and DHA than seeds of a control Brassica plant lacking (i) and/or (ii). In some embodiments, the genomic sequence comprises all or part of the genomic sequence on chromosome N1 between nucleotide positions 8,879,780 and 11,922,690 and the genomic sequence on chromosome N1 between nucleotide positions 22,823,086 and 24,045,492. In some embodiments, the genomic sequence comprises all or part of the genomic sequence on chromosome N1 between nucleotide positions 8,879,780 and 11,922,690 and the genomic sequence on chromosome N6 between nucleotide positions 19,156,645 and 20,846,412. In some embodiments, the genomic sequence comprises all or part of the genomic sequence on chromosome N1 between nucleotide positions 22,823,086 and 24,045,492 and the genomic sequence on chromosome N6 between nucleotide positions 19,156,645 and 20,846,412. In some embodiments, the genomic sequence can include from 25 to 50, 25 to 100, 50 to 200, 100 to 500, 250 to 1,000, 500 to 5,000, 2,000 to 10,000, 5,000 to 20,000, 10,000 to 100,000, 50,000 to 400,000, 25,000 to 1,000,000, 100,000 to 1,000,000, 200,000 to 1,000,000, or 500 to 1,000,000 contiguous nucleotides of the genomic sequence of the B. napus parent genome.

The genomic sequence on chromosome N1 between nucleotide positions 8,879,780 and 11,922,690 can include a single nucleotide polymorphism (SNP) at a position selected from the group 8,952,616, 9,040,901, 9,046,609, 9,048,617, 9,136,686, 9,143,608, 9,248,592, 9,347,120, 9,352,326, 9,454,361, 9,549,523, 9,641,936, 9,652,028, 9,794,198, 9,847,417, 9,921,975, 9,952,792, 10,052,015, 10,402,684, 10,425,211, 10,558,464, 10,613,015, 10,659,284, 10,706,805, 10,748,492, 10,852,010, 11,007,740, 11,047,958, 11,150,929, 11,269,217, 11,343,118, 11,455,979, 11,565,970, 11,659,776, 11,726,807, 11,850,103, and 11,956,477. In some embodiments, the genomic sequence includes 5, 10, 15, 20, 30, 35, or 40 SNPs at different positions selected from the group 8,952,616, 9,040,901, 9,046,609, 9,048,617, 9,136,686, 9,143,608, 9,248,592, 9,347,120, 9,352,326, 9,454,361, 9,549,523, 9,641,936, 9,652,028, 9,794,198, 9,847,417, 9,921,975, 9,952,792, 10,052,015, 10,402,684, 10,425,211, 10,558,464, 10,613,015, 10,659,284, 10,706,805, 10,748,492, 10,852,010, 11,007,740, 11,047,958, 11,150,929, 11,269,217, 11,343,118, 11,455,979, 11,565,970, 11,659,776, 11,726,807, 11,850,103, and 11,956,477.

The genomic sequence on chromosome N1 between nucleotide positions 8,879,780 and 11,922,690 can include at least one SNP at a position selected from the group 9,136,686, 9,641,936, 10,613,015, 9,040,901, 9,048,617, 9,352,326, 9,921,975, and 10,706,805. In some embodiments, the genomic sequence includes 2, 3, 4, 5, 6, 7, or 8 SNPs at different positions selected from the group consisting of 9,136,686, 9,641,936, 10,613,015, 9,040,901, 9,048,617, 9,352,326, 9,921,975, and 10,706,805.

The genomic sequence on chromosome N1 between nucleotide positions 22,823,086 and 24,045,492 can include a SNP at a position selected from the group 22,823,086, 22,880,595, 22,902,670, 22,949,738, 23,011,207, 23,044,228, 23,099,592, 23,176,771, 23,201,595, 23,257,618, 23,302,268, 23,367,822, 23,380,089, 23,457,696, 23,520,607, 23,552,773, 23,598,941, 23,670,623, 23,682,848, 23,745,365, 23,792,572, 23,855,829, 23,910,029, 23,947,522, 24,021,883. In some embodiments, the genomic sequence includes 5, 10, 15, 20, 25, or 30 SNPs at different positions selected from the group 22,823,086, 22,880,595, 22,902,670, 22,949,738, 23,011,207, 23,044,228, 23,099,592, 23,176,771, 23,201,595, 23,257,618, 23,302,268, 23,367,822, 23,380,089, 23,457,696, 23,520,607, 23,552,773, 23,598,941, 23,670,623, 23,682,848, 23,745,365, 23,792,572, 23,855,829, 23,910,029, 23,947,522, 24,021,883, 24,056,999.

The genomic sequence on chromosome N1 between nucleotide positions between nucleotide positions 22,823,086 and 24,045,492 can include a SNP at a position selected from the group 23,089,542, 23,089,635, 23,090,743, 23,090,785, 23,091,367, 23,092,042, 23,150,402, 23,150,595, 23,155,220, 23,155,766, 23,314,197, 23,318,357, 23,343,089, 23,679,276, 23,679,287, 23,679,396, 23,886,929, 23,925,895, 23,963,309, 24,029,270, 24,029,279, 24,029,294. In some embodiments, the genomic sequence includes 5, 10, 15, 20, or 25 SNPs at different positions selected from the group 23,089,542, 23,089,635, 23,090,743, 23,090,785, 23,091,367, 23,092,042, 23,150,402, 23,150,595, 23,155,220, 23,155,766, 23,314,197, 23,318,357, 23,343,089, 23,679,276, 23,679,287, 23,679,396, 23,886,929, 23,925,895, 23,963,309, 24,029,270, 24,029,279, 24,029,294.

The genomic sequence on chromosome N6 between nucleotide positions 19,156,645 and 20,846,412 can include a SNP at a position selected from the group 19,156,645, 19,199,109, 19,325,186, 19,402,086, 19,513,420, 19,583,431, 19,601,021, 19,706,563, 19,800,643, 19,906,666, 20,000,119, 20,095,002, 20,205,211, 20,300,571, 20,406,148, 20,407,023, 20,505,840, 20,601,198, 20,631,917, and 20,702,631. In some embodiments, the genomic sequence includes at least 5, 10, 15, 20, 25, 30 SNPs at different positions selected from the group 19,156,645, 19,199,109, 19,325,186, 19,402,086, 19,513,420, 19,583,431, 19,601,021, 19,706,563, 19,800,643, 19,906,666, 20,000,119, 20,095,002, 20,205,211, 20,300,571, 20,406,148, 20,407,023, 20,505,840, 20,601,198, 20,631,917, and 20,702,631.

The genomic sequence on chromosome N6 between nucleotide positions 19,156,645 and 20,846,412 can include a SNP at a position selected from the group 19,336,744, 19,336,819, 19,337,615, 19,350,156, 19,353,584, 19,353,648, 19,353,749, 19,476,836, 19,783,834, 19,784,007, 19,784,367, 19,784,633, 19,784,672, 19,784,688, 19,784,733, 19,800,525, 20,191,826, 20,300,548, 20,375,643, 20,766,637, 20,769,461, 20,770,769, 20,823,998, 20,825,959, 20,826,301, 20,827,570, 20,827,573.

In some embodiments, the genomic sequence includes at least 5, 10, 15, 20, 25, 30, 35, or 40 SNPs at different positions selected from the group 19,336,744, 19,336,819, 19,337,615, 19,350,156, 19,353,584, 19,353,648, 19,353,749, 19,476,836, 19,783,834, 19,784,007, 19,784,367, 19,784,633, 19,784,672, 19,784,688, 19,784,733, 19,800,525, 20,191,826, 20,300,548, 20,375,643, 20,766,637, 20,769,461, 20,770,769, 20,823,998, 20,825,959, 20,826,301, 20,827,570, 20,827,573.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. he materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic of the different enzymatic activities leading to the production of ARA, EPA and DHA.

FIG. 2A-FIG. 2D show the distribution of EPA, DPA and DHA contents from 279 Brassica accessions. Arrow shows the average content from the PUFA donor line, Kumily LBFLFK.

FIG. 3 is a Manhattan plot showing two genomic blocks on N01 for EPA.

FIG. 4 is a Manhattan plot showing the genomic block on N01 for DPA.

FIG. 5 is a Manhattan plot showing the two genomic blocks on N01 for DHA.

FIG. 6 is a Manhattan plot showing the genomic block on N06 for EPA.

### DETAILED DESCRIPTION

As described herein, Brassica plants can be produced in which the seeds from these plants yield oils with elevated levels of long chain polyunsaturated fatty acids. The term “polyunsaturated fatty acids (PUFA)” as used herein refers to fatty acids comprising at least two (e.g., at least three, four, five or six) double bonds in a fatty acid chain that is, for example, from 18 to 24 carbon atoms in length. In some embodiments, the term relates to very long chain PUFA (VLC-PUFA) having from 20 to 24 carbon atoms in the fatty acid chain. PUFAs can be, for example, dihomo-gamma linolenic acid (DHGLA, 20:3 (8,11,14)), arachidonic acid (ARA, 20:4 (5,8,11,14)), EPA (20:5 (5,8,11,14,17)), docosapentaenoic acid (DPA, 22:5 (4,7,10,13,16)), DHA (22:6 (4,7,10,13,16,19)), and/or eicosatetraenoic acid (ETA, 20:4 (8,11,14,17)). In some embodiments, seeds of Brassica plants provided herein can produce higher levels of EPA, higher levels of DPA, higher levels of DHA, higher levels of ARA, higher levels of EPA and DHA, higher levels of DHA and DPA, higher levels of DPA and EPA, higher levels of ARA, EPA, and DHA, or higher levels of EPA, DHA, and DPA.

In some embodiments, the Brassica plants or parts thereof can produce one or more intermediates of VLC-PUFA which occur during synthesis. Such intermediates can be formed from substrates by one or more activities of a desaturase, keto-acyl-CoA-synthase, keto-acyl-CoA-reductase, dehydratase, or enoyl-CoA-reductase polypeptide. In some embodiments, substrates can be linoleic acid (LA, 18:2 (9,12)), gamma linolenic acid (GLA 18:3 (6,9,12)), DHGLA, ARA, eicosadienoic acid 20:2 (11,14), ETA, or EPA.

In some embodiments, a Brassica plant or part thereof provided herein can be produced by crossing a first Brassica plant with a second Brassica plant and selecting progeny. In some embodiments, the first Brassica plant can include one or more expression cassettes comprising at least one polynucleotide sequence encoding one or more desaturases and/or one or more elongases. In some embodiments, the second Brassica plant contributes at least one genomic sequence that is in part or in whole responsible for the higher levels of DHA and/or EPA.

The term “polynucleotide” according to the present disclosure refers to a deoxyribonucleic acid or ribonucleic acid. Unless stated otherwise, “polynucleotide” herein refers to a single strand of a DNA polynucleotide or to a double stranded DNA polynucleotide. As used herein, the terms nucleotide/polynucleotide and nucleotide sequence/polynucleotide sequence are used interchangeably, and such terms encompass both double stranded and single stranded nucleic acids.

The term “desaturase” encompasses all enzymatic activities and enzymes catalyzing the desaturation of fatty acids with different lengths and numbers of unsaturated carbon atom double bonds. For example, a desaturase can be a delta 4 (d4)-desaturase that catalyzes the dehydrogenation of the 4th and 5th carbon atom; a delta 5 (d5)-desaturase catalyzing the dehydrogenation of the 5th and 6th carbon atom; a delta 6 (d6)-desaturase catalyzing the dehydrogenation of the 6th and 7th carbon atom; a delta 8 (d8)-desaturase catalyzing the dehydrogenation of the 8th and 9th carbon atom; a delta 9 (d9)-desaturase catalyzing the dehydrogenation of the 9th and 10th carbon atom; a delta 12 (d12)-desaturase catalyzing the dehydrogenation of the 12th and 13th carbon atom; or a delta 15 (d15)-desaturase catalyzing the dehydrogenation of the 15th and 16th carbon atom.

The terms “elongase” encompasses all enzymatic activities and enzymes catalyzing the elongation of fatty acids with different lengths and numbers of unsaturated carbon atom double bonds. In some embodiments, the term “elongase” refers to the activity of an elongase that introduces two carbon molecules into the carbon chain of a fatty acid.

In some embodiments, the one or more expression cassettes can have polynucleotide sequences encoding one or more d5Des, one or more d6Elo, one or more d5Des, one or more o3Des, one or more d5Elo and one or more d4Des, for example, for at least one CoA-dependent D4Des and one phospholipid-dependent d4Des. In some embodiments, one or more d12Des also are encoded.

In some embodiments, the one or more expression cassettes can have polynucleotide sequences encoding at least two d6Des, at least two d6Elo, and/or at least two o3Des. In some embodiments, the one or more expression cassettes also can encode at least one CoA-dependent d4Des and at least one phospholipid dependent d4Des.

Polynucleotides encoding polypeptides that exhibit delta-6-elongase activity have been described, for example, in WO2001/059128, WO2004/087902, WO2005/012316, and WO 2015/089587, which are incorporated herein in their entirety. Non-limiting exemplary delta-6-elongases include those from Physcomitrella patens and Pyramimonas cordata.

Polynucleotides encoding polypeptides which exhibit delta-5-desaturase (d5Des) activity have been described, for example, in WO2002/026946, WO2003/093482, and WO 2015/089587, which are incorporated herein in their entirety. Non-limiting exemplary delta-5-desaturases include those from Thraustochytrium sp., Pavlova salina, and Pyramimonas cordata.

Polynucleotides encoding polypeptides which exhibit delta-6-desaturase activity have been described in WO2005/012316, WO2005/083093, WO2006/008099 and WO2006/069710, and WO 2015/089587, which are incorporated herein in their entirety. Non-limiting exemplary delta-6-desaturases include those from Ostreococcus tauri, Micromonas pusilla, and Osreococcus lucimarinus.

Polynucleotides encoding polypeptides which exhibit delta-5-elongase activity have been described in WO2005/012316, WO2005/007845, WO2007/096387, WO2006/069710, and WO 2015/089587, which are incorporated herein in their entirety. Non-limiting exemplary delta-5-elongases include those from Ostreococcus tauri and Pyramimonas cordata.

Polynucleotides encoding polypeptides which exhibit delta-12-desaturase activity have been described for example in WO2006100241 and WO 2015/089587, which are incorporated herein in their entirety. Non-limiting exemplary delta-12-desaturases include those from Phytophthora sojae and Lachancea kluyveri.

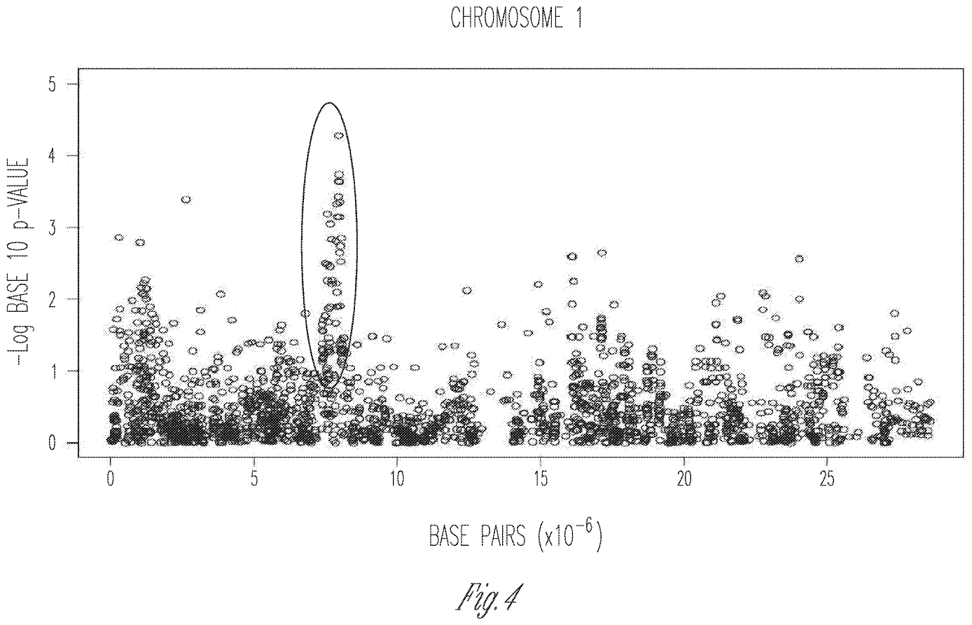

Polynucleotides encoding polypeptides which exhibit delta-4-desaturase (d4Des) activity have been described for example in WO2004/090123, WO2002026946, WO2003078639, WO2005007845, and WO 2015/089587, which are incorporated herein in their entirety. Non-limiting exemplary delta-4-desaturases include those from Euglena gracilis, Thraustochytrium sp., Pavlova lutheri, and Pavlova salina. See, e.g., delta-4 desaturase “PIDES 1” and FIGS. 3a-3d of WO2003078639 and FIGS. 3a, 3b of WO2005007845, respectively.

Polynucleotides encoding polypeptides which exhibit omega 3-desaturase (o3Des) activity have been described for example in WO2008/022963, WO2005012316, WO2005083053, and WO 2015/089587, which are incorporated herein in their entirety. Non-limiting exemplary omega-3-desaturases include those from Phytium irregular, Phytophthora infestans, and Pichia pastoris.

Polynucleotides encoding polypeptides which exhibit delta-15-desaturase activity have been described for example in WO2010/066703, which is incorporated herein in its entirety. Non-limiting exemplary delta-15 destaurases include the delta-15 desaturase from Cochliobolus heterostrophus C5.

Additional polynucleotides that encode polypeptides having desaturase or elongase activities as specified above can be obtained from various organisms, including but not limited to, organisms of genus Ostreococcus, Thraustochytrium, Euglena, Thalassiosira, Phytophthora, Phytium, Cochliobolus, or Physcomitrella. Orthologs, paralogs or other homologs having suitable desaturase or elongase activities may be identified from other species. In some embodiments, such orthologs, paralogs, or homologs are obtained from plants such as algae, for example Isochrysis, Mantoniella, or Crypthecodinium, algae/diatoms such as Phaeodactylum, mosses such as Ceratodon, or higher plants such as the Primulaceae such as Aleuritia, Calendula stellata, Osteospermum spinescens or Osteospermum hyoseroides, microorganisms such as fungi, such as Aspergillus, Entomophthora, Mucor or Mortierella, bacteria such as Shewanella, yeasts or animals. Non-limiting exemplary animals are nematodes such as Caenorhabditis, insects or vertebrates. Among the vertebrates, the nucleic acid molecules may, in some embodiments, be derived from Euteleostomi, Actinopterygii; Neopterygii; Teleostei; Euteleostei, Protacanthopterygii, Salmoniformes; Salmonidae or Oncorhynchus, such as from the order of the Salmoniformes, such as the family of the Salmonidae, such as the genus Salmo, for example from the genera and species Oncorhynchus mykiss, Trutta trutta or Salmo trutta fario. Moreover, the nucleic acid molecules may be obtained from the diatoms such as the genera Thalassiosira or Phaeodactylum.

The term “polynucleotide” as used herein further encompasses variants, muteins or derivatives of the aforementioned specific polynucleotides that are suitable for use in embodiments of the present disclosure.

Nucleic acid variants or derivatives according to the disclosure are polynucleotides which differ from a given reference polynucleotide by at least one nucleotide substitution, addition and/or deletion. If the reference polynucleotide codes for a protein, the function of this protein is conserved in the variant or derivative polynucleotide, such that a variant nucleic acid sequence shall still encode a polypeptide having a desaturase or elongase activity as specified above. Variants or derivatives also encompass polynucleotides comprising a nucleic acid sequence which is capable of hybridizing to the aforementioned specific nucleic acid sequences, for example, under stringent hybridization conditions. These stringent conditions are known in the art and can be found, for example, in Current Protocols in Molecular Biology, John Wiley & Sons, N. Y. (1989), 6.3.1-6.3.6. A non-limiting example for stringent hybridization conditions are hybridization conditions in 6× sodium chloride/sodium citrate (SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1 to 5′ SSC (pH 7.2). If organic solvent is present in the abovementioned buffer, for example 50% formamide, the temperature under standard conditions is approximately 42° C. The hybridization conditions for DNA: DNA hybrids are, for example, 0.1×SSC and 20° C. to 45° C., such between 30° C. and 45° C. The hybridization conditions for DNA:RNA hybrids are, for example, 0.1×SSC and 30° C. to 55° C., such as between 45° C. and 55° C. The abovementioned hybridization temperatures are determined for example for a nucleic acid with approximately 100 bp (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA, i.e. using degenerated primers against conserved domains of the polypeptides of the present disclosure. Conserved domains of a polypeptide suitable for use in embodiments of the present disclosure may be identified by a sequence comparison of the nucleic acid sequences of the polynucleotides or the amino acid sequences of the polypeptides of the present disclosure. Oligonucleotides suitable as PCR primers as well as suitable PCR conditions are described in the accompanying Examples. As a template, DNA or cDNA from bacteria, fungi, plants or animals may be used. Further, variants include polynucleotides comprising nucleic acid sequences which are at least 50%, at least 55%, at least 60%, at least 65%, at least 70%, at least 75%, at least 80%, at least 85%, at least 90%, at least 95%, at least 98% or at least 99% identical to the nucleic acid coding sequences shown in any one of the T-DNA sequences. Of course, the variants must retain the function of the respective enzyme, i.e., a variant of a delta-4-desaturase must retain delta-4-desaturase activity.

The percent identity values are, in some embodiments, calculated over the entire amino acid or nucleic acid sequence region. A series of programs based on a variety of algorithms is available to the skilled worker for comparing different sequences. In some embodiments, the percent identity between two amino acid sequences is determined using the Needleman and Wunsch algorithm (Needleman 1970, J. Mol. Biol. (48):444-453) which has been incorporated into the needle program in the EMBOSS software package (EMBOSS: The European Molecular Biology Open Software Suite, Rice,P., Longden,I., and Bleasby,A, Trends in Genetics 16(6), 276-277, 2000), a BLOSUM62 scoring matrix, and a gap opening penalty of 10 and a gap extension penalty of 0.5. Non-limiting example of parameters to be used for aligning two amino acid sequences using the needle program are the default parameters, including the EBLOSUM62 scoring matrix, a gap opening penalty of 10 and a gap extension penalty of 0.5. In yet another embodiment, the percent identity between two nucleotide sequences is determined using the needle program in the EMBOSS software package (EMBOSS: The European Molecular Biology Open Software Suite, Rice, P., Longden, I., and Bleasby, A, Trends in Genetics 16(6), 276-277, 2000), using the EDNAFULL scoring matrix and a gap opening penalty of 10 and a gap extension penalty of 0.5. A non-limiting example of parameters to be used in conjunction for aligning two nucleic acid sequences using the needle program are the default parameters, including the EDNAFULL scoring matrix, a gap opening penalty of 10 and a gap extension penalty of 0.5. The nucleic acid and protein sequences of the present disclosure can further be used as a “query sequence” to perform a search against public databases to, for example, identify other family members or related sequences. Such searches can be performed using the BLAST series of programs (version 2.2) of Altschul et al. (Altschul 1990, J. Mol. Biol. 215:403-10). BLAST using desaturase and elongase nucleic acid sequences of the disclosure as query sequence can be performed with the BLASTn, BLASTx or the tBLASTx program using default parameters to obtain either nucleotide sequences (BLASTn, tBLASTx) or amino acid sequences (BLASTx) homologous to desaturase and elongase sequences of the disclosure. BLAST using desaturase and elongase protein sequences of the disclosure as query sequence can be performed with the BLASTp or the tBLASTn program using default parameters to obtain either amino acid sequences (BLASTp) or nucleic acid sequences (tBLASTn) homologous to desaturase and elongase sequences of the disclosure. To obtain gapped alignments for comparison purposes, Gapped BLAST using default parameters can be utilized as described in Altschul et al. (Altschul 1997, Nucleic Acids Res. 25(17):3389-3402).

The variant polynucleotides or fragments referred to above, in some embodiments, encode polypeptides retaining desaturase or elongase activity to a significant extent, such as at least 10%, at least 20%, at least 30%, at least 40%, at least 50%, at least 60%, at least 70%, at least 80%, at least 90%, or at least 95% of the desaturase or elongase activity exhibited by any of the polypeptide comprised in any of the T-DNAs disclosed herein.

Further enzymes that may be used in embodiments of the present disclosure include, but are not limited to, acyltransferases and transacylases (see, for example, WO 2011161093), such as, for example, lysophosphatidic acid acyltransferase (LPAAT), diacylglycerol acyltransferase (DGAT), phospholipid diacylglycerol acyltransferase (PDAT), diacylglyceroldiacylglycerol transacylase (DDAT), and lysophospholipid acyltransferase (LPLAT). LPLATs can have activity as lysophosphophatidylethanolamine acyltransferase (LPEAT) and lysophosphatidylcholine acyltransferase (LPCAT).

The term “expression control sequence” as used herein refers to a nucleic acid sequence which is capable of governing, i.e., initiating and controlling, transcription of a nucleic acid sequence of interest, in the present case the nucleic sequences recited above. Such a sequence usually comprises or consists of a promoter or a combination of a promoter and enhancer sequences. Expression of a polynucleotide comprises transcription of the nucleic acid molecule, for example, into a translatable mRNA. Additional regulatory elements may include transcriptional as well as translational enhancers. The following promoters and expression control sequences may be, for example, used in an expression vector according to the present disclosure. The cos, tac, trp, tet, trp-tet, lpp, lac, lpp-lac, lacIq, T7, T5, T3, gal, trc, ara, SP6, λ-PR or λ-PL promoters are, for example, used in Gram-negative bacteria. In some embodiments, for Gram-positive bacteria, promoters amy and SPO2 may be used. In some embodiments, yeast or fungal promoters ADC1, AOX1r, GAL1, MFα, AC, P-60, CYC1, GAPDH, TEF, rp28, ADH may be used. In some embodiments, for animal cell or organism expression, the promoters CMV-, SV40-, RSV-promoter (Rous sarcoma virus), CMV-enhancer, SV40-enhancer may be used. From plants the promoters CaMV/35S (Franck 1980, Cell 21: 285-2941, PRP1 (Ward 1993, Plant. Mol. Biol. 22), SSU, OCS, lib4, usp, STLS1, B33, nos or the ubiquitin or phaseolin promoter. In some embodiments, inducible promoters may be used, such as the promoters described in EP 0388186 A1 (i.e. a benzylsulfonamide-inducible promoter), Gatz 1992, Plant J. 2:397-404 (i.e. a tetracyclin-inducible promoter), EP 0335528 A1 (i.e. an abscisic-acid-inducible promoter) or WO 93/21334 (i.e., an ethanol- or cyclohexenol-inducible promoter). Further suitable plant promoters are the promoter of cytosolic FBPase or the ST-LSI promoter from potato (Stockhaus 1989, EMBO J. 8, 2445), the phosphoribosyl-pyrophosphate amidotransferase promoter from Glycine max (Genbank accession No. U87999) or the node-specific promoter described in EP 0249676 A1. In some embodiments, promoters which enable the expression in tissues which are involved in the biosynthesis of fatty acids are used. In some embodiments, seed-specific promoters are used, such as the USP promoter in accordance with the practice, but also other promoters such as the LeB4, DC3, phaseolin or napin promoters. In some embodiments, seed-specific promoters which can be used for monocotyledonous or dicotyledonous plants and which are described in U.S. Pat. No. 5,608,152 (napin promoter from oilseed rape), WO 98/45461 (oleosin promoter from Arobidopsis, U.S. Pat. No. 5,504,200 (phaseolin promoter from Phaseolus vulgaris), WO 91/13980 (Bce4 promoter from Brassica), by Baeumlein et al., Plant J., 2, 2, 1992:233-239 (LeB4 promoter from a legume), these promoters being suitable for dicots, may be used. The following promoters are suitable for monocots: lpt-2 or lpt-1 promoter from barley (WO 95/15389 and WO 95/23230), hordein promoter from barley and other promoters which are suitable, and which are described in WO 99/16890. In principle, it is possible to use all-natural promoters together with their regulatory sequences, such as those mentioned above, for the novel process. Likewise, it is possible and advantageous to use synthetic promoters, either additionally or alone, especially when they mediate a seed-specific expression, such as, for example, as described in WO 99/16890. In a particular embodiment, seed-specific promoters are utilized to enhance the production of the desired PUFA or VLC-PUFA.

The term “operatively linked” as used herein means that the expression control sequence and the nucleic acid of interest are linked so that the expression of the said nucleic acid of interest can be governed by the said expression control sequence, i.e. the expression control sequence shall be functionally linked to the said nucleic acid sequence to be expressed. Accordingly, the expression control sequence and, the nucleic acid sequence to be expressed may be physically linked to each other, e.g., by inserting the expression control sequence at the 5′end of the nucleic acid sequence to be expressed. Alternatively, the expression control sequence and the nucleic acid to be expressed may be merely in physical proximity so that the expression control sequence is capable of governing the expression of at least one nucleic acid sequence of interest. The expression control sequence and the nucleic acid to be expressed are, in some embodiments, separated by not more than 500 bp, 300 bp, 100 bp, 80 bp, 60 bp, 40 bp, 20 bp, 10 bp or 5 bp.

Polynucleotides of the present disclosure can include, in addition to a promotor, a terminator sequence operatively linked to polynucleotides which encode the enzymes, e.g., the desaturases and/or elongases, described herein.

The term “terminator” as used herein refers to a nucleic acid sequence which is capable of terminating transcription. These sequences will cause dissociation of the transcription machinery from the nucleic acid sequence to be transcribed. In some embodiments, the terminator shall be active in plants and, in particular, in plant seeds. Suitable terminators are known in the art and include polyadenylation signals such as the SV40-poly-A site or the tk-poly-A site or one of the plant specific signals indicated in Loke et al. (Loke 2005, Plant Physiol 138, pp. 1457-1468), downstream of the nucleic acid sequence to be expressed.

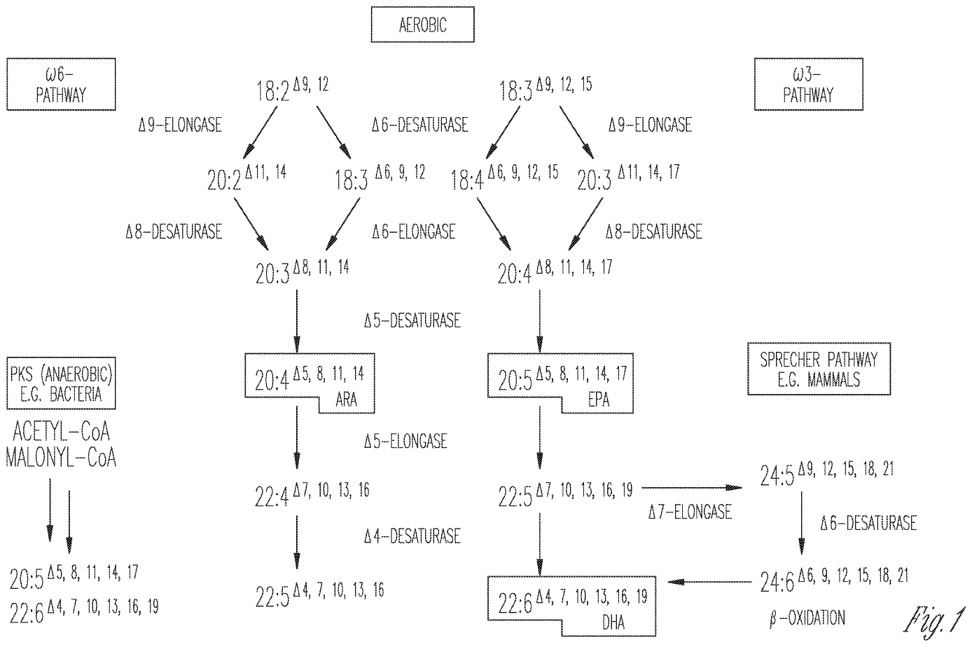

Recombinant nucleic acid molecules that encode desaturases and elongases described in FIG. 1 are suitable for use in embodiments of the present disclosure. As used herein, “recombinant” means the combination of nucleic acid sequences using techniques available to those of ordinary skill in molecular biology, to produce one or more expression cassette(s) (alternatively designated herein as gene constructs) or one or more vector(s) comprising polynucleotides encoding the desaturases and elongases described in FIG. 1, which are operably linked with expression control sequences such as promoters, to effect expression of the desaturase and elongase polynucleotides in a host cell.

Disclosed herein are recombinant polynucleotides (such as T-DNAs) for expression of desaturases and elongases in a Brassica plant. In some embodiments, a T-DNA comprises a left and a right border element and at least one expression cassette comprising a promotor, operatively linked to polynucleotides encoding various combinations of the desaturases and elongases, and downstream thereof other regulatory elements including but not limited to a terminator.

A “T-DNA” as used herein is a nucleic acid capable of eventual integration into the genetic material (genome) of a Brassica plant through transformation using methods available to those skilled in the art of molecular biology.

For example, a T-DNA suitable for use in embodiments of the present disclosure may be comprised in a circular nucleic acid, e.g. a plasmid, such that an additional nucleic acid section is present between the left and right border elements. The additional nucleic acid section may include one or more genetic elements for replication of the total nucleic acid, i.e. the nucleic acid molecule comprising the T-DNA and the additional nucleic acid section, in one or more host microorganisms, for example, in a microorganism of genus Escherichia, such as E. coli, and/or Agrobacterium. Such circular nucleic acids comprising a T-DNA of the present disclosure are particularly useful as transformation vectors.

In some embodiments, the T-DNA length is sufficiently large to introduce a number of enzymes, e.g. desaturase and elongase, genes in the form of expression cassettes, such that each individual gene is operably liked to at least one promotor and at least one terminator, as is shown in the examples below.

A T-DNA can comprise the coding sequences of one or more single genes. For example, T-DNA comprising the coding sequences of one or more single genes can be combined with other T-DNA comprising one or more other genes. The T-DNAs suitable for use in embodiments of the present disclosure may comprise one or more expression cassettes encoding for one or more d5Des, one or more d6Elo, one or more d5Des, one or more o3Des, one or more d5Elo and one or more d4Des, for example, for at least one CoA-dependent D4Des and one phospholipid-dependent d4Des. In some embodiments, the T-DNA encodes also one or more d12Des.

In one embodiment, the Brassica plant of the present disclosure or a part thereof (e.g., root, stem, leave, seed, flower, cell etc.) comprises one or more T-DNAs which encode for at least two d6Des, at least two d6Elo, and/or at least two o3Des. In one embodiment, the Brassica plant or a part thereof described herein includes a T-DNA comprising one or more expression cassettes encoding at least one CoA-dependent d4Des and at least one phospholipid dependent d4Des.

In one embodiment, at least one T-DNA suitable for use comprises an expression cassette which encodes at least one d12Des. In one embodiment, the T-DNA or T-DNAs comprise one or more expression cassettes encoding one or more one or more d5Des (e.g., delta 5 desaturase from Thraustochytrium sp., Tc_GA), o3Des (e.g., omega 3 desaturase from Pythium irregular, Pir_GA), d6Elo (delta 6 elongase from Thalassiosira pseudonana, Tp_GA) and/or d6Elo (e.g., delta-6 elongase from Physcomitrella patens, Pp_GA).

According to the disclosure, the T-DNA may also comprise, instead of one or more of the coding sequences discussed herein, a functional homolog thereof. A functional homolog of a coding sequence is a sequence coding for a polypeptide having the same metabolic function as the replaced coding sequence. As a non-limiting example, a functional homolog of a delta-5-desaturase would be another delta-5-desaturase, and a functional homolog of a delta-5-elongase would be another delta-5-elongase. A functional homolog of a plant seed specific promotor is another plant seed specific promotor. The functional homolog of a terminator, correspondingly, is a sequence for ending transcription of a nucleic acid sequence.

Certain T-DNA sequences suitable for use in embodiments of the present disclosure are described in PCT/EP2015/076632 (published as WO/2016/075327).

In some embodiments, constructs comprising a T-DNA vector comprising certain desaturases and elongases described herein can be transformed into a plant cell by microorganism-mediated transformation, for example, by Agrobacterium-mediated transformation. In some embodiments, the microorganism is a disarmed strain of genus Agrobacterium, such as species Agrobacterium tumefaciens or species Agrobacterium rhizogenes. Suitable Agrobacterium strains for use are for example described in WO06024509A2, and methods for plant transformation using such microorganisms are for example described in WO13014585A1, incorporated herein by reference.

The term “vector” encompasses phage, plasmid, viral vectors as well as artificial chromosomes, such as bacterial or yeast artificial chromosomes. Moreover, the term also relates to targeting constructs which allow for random or site-directed integration of the targeting construct into genomic DNA. Such target constructs, in some embodiments, comprise DNA of sufficient length for either homolgous or heterologous recombination as described in detail below. The vector suitable for use in some embodiments further comprises selectable markers for propagation and/or selection in a host. The vector may be incorporated into a host cell by various techniques well known in the art. It is to be understood that the vector may further comprise nucleic acid sequences which allow for homologous recombination or heterologous insertion. Vectors can be introduced into prokaryotic or eukaryotic cells via conventional transformation or transfection techniques. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, natural competence, carbon-based clusters, chemically mediated transfer, electroporation or particle bombardment. Suitable methods for the transformation or transfection of host cells, including plant cells, can be found in Sambrook et al. (Molecular Cloning: A Laboratory Manual, 2nd ed., Cold Spring Harbor Laboratory, Cold Spring Harbor Laboratory Press, Cold Spring Harbor, NY, 1989) and other laboratory manuals, such as Methods in Molecular Biology, 1995, Vol. 44, Agrobacterium protocols, Ed.: Gartland and Davey, Humana Press, Totowa, New Jersey. Alternatively, a plasmid vector may be introduced by heat shock or electroporation techniques. Should the vector be a virus, it may be packaged in vitro using an appropriate packaging cell line prior to application to host cells.

In some embodiments, the vector referred to herein is suitable as a cloning vector, i.e. replicable in microbial systems. Such vectors ensure efficient cloning in bacteria and, in some embodiments, yeasts or fungi and make possible the stable transformation of plants. Those which must be mentioned are, in particular, various binary and co-integrated vector systems which are suitable for the T DNA-mediated transformation. Such vector systems are characterized in that they contain at least the vir genes, which are involved in the Agrobacterium-mediated transformation, and the sequences which delimit the T-DNA (T-DNA border). These vector systems, in some embodiments, also comprise further cis-regulatory regions such as promoters and terminators and/or selection markers with which suitable transformed host cells or organisms can be identified. While co-integrated vector systems have vir genes and T-DNA sequences arranged on the same vector, binary systems are based on at least two vectors, one of which bears vir genes, but no T-DNA, while a second one bears T-DNA, but no vir gene. As a consequence, the last-mentioned vectors are relatively small, easy to manipulate and can be replicated both in E. coli and in Agrobacterium. These binary vectors include vectors from the pBIB-HYG, pPZP, pBecks, pGreen series. In some embodiments, used in accordance with the disclosure are Bin19, pBI101, pBinAR, pGPTV and pCAMBIA. An overview of binary vectors and their use can be found in Hellens et al, Trends in Plant Science (2000)5, 446-451. Furthermore, by using appropriate cloning vectors, the polynucleotides can be introduced into host cells or organisms such as plants or animals and, thus, be used in the transformation of plants, such as those which are published, and cited, in: Plant Molecular Biology and Biotechnology (CRC Press, Boca Raton, Florida), chapter 6/7, pp. 71-119 (1993); F. F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus 1991, Annu. Rev. Plant Physiol. Plant Molec. Biol. 42, 205-225. The binary BAC (BiBAC) vector, suitable for transforming large T-DNAs into plants, is described in U.S. Pat. Nos. 5,733,744 and 5,977,439.

An expression vector, i.e. a vector which comprises the polynucleotide of the disclosure having the nucleic acid sequence operatively linked to an expression control sequence (also called “expression cassette”) allowing expression in prokaryotic plant cells or isolated fractions thereof.

A Brassica plant or seed may comprise, integrated in its genome, a T-DNA capable of effecting expression of polynucleotides expressing the desaturases and elongases, such as the desaturases and elongases described in FIG. 1.

In some embodiments, the plants of the present disclosure are transgenic, i.e. they comprise genetic material not present in corresponding wild type plant or arranged differently in corresponding wild type plant, for example differing in the number of genetic elements. For example, the plants of the present disclosure can comprise promotors also found in wild type plants, but the plants of the present disclosure comprise such promotor operatively linked to a coding sequence such that this combination of promotor and coding sequence is not found in the corresponding wild type plant.

The Brassica plants of the present disclosure may comprise one or more T-DNA(s) described herein comprising expression cassettes which include one or more genes encoding for one or more d5Des, one or more d6Elo, one or more d5Des, one or more o3Des, one or more d5Elo and one or more D4Des, such as for at least one CoA-dependent D4Des and one phospholipid-dependent d4Des. In one embodiment, at least one T-DNA comprises an expression cassette which encodes for at least one d12Des. In one embodiment, the T-DNA or T-DNAs comprise one or more expression cassettes encoding one or more d5Des (e.g., delta 5 desaturase from Thraustochytrium sp., Tc_GA), o3Des (e.g., omega 3 desaturase from Pythium irregular, Pir_GA), d6Elo (delta 6 elongase from Thalassiosira pseudonana, Tp_GA) and/or d6Elo (e.g., delta-6 elongase from Physcomitrella patens, Pp_GA).

Seeds of an event described in the example below have been deposited at ATCC under the provisions of the Budapest treaty on the International Recognition of the Deposit of Microorganisms for the Purposes of Patent Procedure, i.e. seeds of event “LBFLFK”=ATCC Designation “PTA-121703” (LBFLFK as described in PCT/EP2015/076632 (published as WO/2016/075327) and US 20180298400).

In some embodiments, a Brassica plant described herein can be produced using methods described in WO 2004/071467, WO 2015/089587 or WO 2016/075327, for producing Brassica lines. In some embodiments, a Brassica plant described herein can be produced using methods described in U.S. Pat. No. 7,807,849 B2 for producing Arabidopsis lines. In some embodiments, a Brassica plant described herein can be produced using methods described in WO 2013/153404, for producing Camelina lines.

In some embodiments, the Brassica plants provided herein can be a Brassica plant line. The term “line” refers to a group of plants that displays little to no genetic variation for at least one trait among individuals sharing that designation.

The Brassica plants and seeds disclosed herein are, in some embodiments, of a species comprising a genome of one or two members of the species Brassica oleracea, Brassica nigra, and Brassica rapa. In some embodiments, the Brassica plants and seeds disclosed herein are of the species Brassica napus, Brassica carinata, Brassica juncea, Brassica oleracea, Brassica nigra, or Brassica rapa. In some embodiments, the plants and seeds are of the species Brassica napus and Brassica carinata.

In some embodiments, a plant provided herein is a plant found in the “Tiangle of U”, i.e. a plant of genus Brassica: Brassica napus (AA CC genome; n=19), which is an amphidiploid plant of the Brassica genus, but is thought to have resulted from hybridization of Brassica rapa (AA genome; n=10) and Brassica oleracea (CC genome; n=9). Brassica juncea (AA BB genome; n=18) is an amphidiploid plant of the Brassica genus that is generally thought to have resulted from the hybridization of Brassica rapa and Brassica nigra (BB genome; n=8). Under some growing conditions, B. juncea may have certain superior traits to B. napus. These superior traits may include higher yield, better drought and heat tolerance and better disease resistance. Brassica carinata (BB CC genome; n=17) is an amphidiploid plant of the Brassica genus but is thought to have resulted from hybridization of Brassica nigra and Brassica oleracea. Under some growing conditions, B. carinata may have superior traits to B. napus.

In some embodiments, the Brassica plant provided herein is a “canola” plant. Canola herein generally refers to plants of Brassica species that have less than 2% (e.g., less than 1%, 0.5%, 0.2% or 0.1%) erucic acid (delta 13-22:1) by weight in seed oil and less than about 30 micromoles (e.g., less than 30, 25, 20 15, or 10 micromoles) of glucosinolates per gram of oil free meal (meal fraction). Typically, canola oil may include saturated fatty acids known as palmitic acid and stearic acid, a monounsaturated fatty acid known as oleic acid, and polyunsaturated fatty acids known as linoleic acid and linolenic acid. Canola oil may contain less than about 7% (w/w) total saturated fatty acids (mostly palmitic acid and stearic acid) and greater than 40% (w/w) oleic acid (as percentages of total fatty acids). Traditionally, canola crops include varieties of Brassica napus and Brassica rapa. Non-limiting exemplary Brassica plants of the present disclosure are spring canola (Brassica napus subsp. oleifera var. annua) and winter canola (Brassica napus subsp. oleifera var. biennis). Furthermore, a canola quality Brassica juncea variety, which has oil and meal qualities similar to other canola types, has been added to the canola crop family (U.S. Pat. Nos. 6,303,849; 7,423,198; all of which are incorporated herein by reference). Likewise, it is possible to establish canola quality B. carinata varieties by crossing canola quality variants of Brassica napus with Brassica nigra and appropriately selecting progeny thereof, optionally after further back-crossing with B. carinata, B. napus, and/or B. nigra.

This method allows to effectively incorporate genetic material of other members of family Brassicaceae, such as genus Brassica, into the genome of a plant comprising a T-DNA disclosed herein. The method is particularly useful for combining an event comprising a T-DNA with genetic material responsible for beneficial traits exhibited in other members of family Brassicaceae. Beneficial traits of other members of family Brassicaceae are exemplarily described herein, other beneficial traits or genes and/or regulatory elements involved in the manifestation of a beneficial trait may be described elsewhere. In some embodiments, a Brassica plant that produces higher levels of linolenic acid can be crossed with a Brassica plant that produces one or more of EPA, DPA, and DHA, such that the progeny produces higher levels of one or more of EPA, DPA and/or DHA than either parent plant. In some embodiments, a Brassica plant that produces low levels of linolenic acid can be crossed with a Brassica plant that produces one or more of EPA, DPA and/or DHA, and surprisingly, the progeny can produce higher levels of one or more of EPA, DPA and/or DHA than either parent plant. In some embodiments, a Brassica plant that produces higher levels of linoleic acid can be crossed with a Brassica plant that produces one or more of EPA, DPA and/or DHA, such that the progeny produces higher levels of one or more of EPA, DPA and/or DHA than either parent plant. In some embodiments, a Brassica plant that produces low levels of linoleic acid can be crossed with a Brassica plant that produces one or more of EPA, DPA and/or DHA, and surprisingly, the progeny can produce higher levels of one or more of EPA, DPA and/or DHA than either parent plant. In some embodiments, a Brassica plant that produces mid-range levels of linoleic acid can be crossed with a Brassica plant that produces one or more of EPA, DPA and/or DHA, and surprisingly, the progeny can produce higher levels of one or more of EPA, DPA and/or DHA than either parent plant, and in some embodiments, higher levels of DHA and/or EPA than a plant resulting from a cross of a high linoleic acid producing Brassica parent with a Brassica plant that produces one or more of EPA, DPA and/or DHA.

In some embodiments, the parent plant not comprising the T-DNA described herein is a parent that produces high linolenic acid, such as the rrm1367-003 line described in WO2015/066082. In some embodiments, the parent plant not comprising the T-DNA described herein can be a parent that produces low linolenic acid. In some embodiments, the parent plant not comprising the T-DNA described herein is a parent that produces low linoleic acid.

In some embodiments, a parent plant can have all or part of at least one genomic sequence of a B. napus parent genome that confers higher PUFA content, where the genomic sequence is selected from the group consisting of: a) the genomic sequence on chromosome N1 between nucleotide positions 8879780 and 11922690; b) the genomic sequence on chromosome N1 between nucleotide positions 22823086 and 24045492; and c) the genomic sequence on chromosome N6 between nucleotide positions 19156645 and 20846412. In the present disclosure, nucleotide positions within a given chromosome are based on the position in the genomic sequence of Brassica napus cultivar DH12075.

In some embodiments, a Brassica plant produced as described herein comprises (i) a T-DNA as described herein and (ii) all or part of at least one genomic sequence of a B. napus parent genome that confers higher PUFA content, where the genomic sequence is selected from the group consisting of: a) the genomic sequence on chromosome N1 between nucleotide positions 8879780 and 11922690; b) the genomic sequence on chromosome N1 between nucleotide positions 22823086 and 24045492; and c) the genomic sequence on chromosome N6 between nucleotide positions 19156645 and 20846412.

In some embodiments, the genomic sequence of a B. napus parent genome that confers higher PUFA can include, for example, from 25 to 50, 25 to 100, 50 to 200, 100 to 500, 250 to 1,000, 500 to 5,000, 2,000 to 10,000, 5,000 to 20,000, 10,000 to 100,000, 50,000 to 400,000, 25,000 to 1,000,000, 100,000 to 1,000,000, 200,000 to 1,000,000, or 500 to 1,000,000 contiguous nucleotides or longer of a region of chromosome N1 (e.g., the genomic sequence on chromosome N1 between nucleotide positions 8879780 and 11922690 and/or the genomic sequence on chromosome N1 between nucleotide positions 22823086 and 24045492) and/or a region of chromosome N6 (e.g., the genomic sequence on chromosome N6 between nucleotide positions 19156645 and 20846412).

In some embodiments, one or more single nucleotide polymorphisms (SNPs) can be present in all or part of at least one genomic sequence of a B. napus parent genome that confers higher PUFA content. The presence of one or more such SNPs can be used in selecting suitable parents and progeny. A SNP can occur within coding and non-coding regions, including exons, introns, and untranslated sequences. Examples of SNPs include substitutions of one or more nucleotides, deletions of one or more nucleotides, and insertions of one or more nucleotides. In some embodiments, a nucleotide substitution can be a transition, in which a purine nucleotide is substituted for another purine (e.g., A to G or G to A), or a pyrimidine nucleotide is substituted for another pyrimidine (e.g., C to T or T to C). In some embodiments, a nucleotide substitution can be a transversion, in which a purine nucleotide is substituted for a pyrimidine or a pyrimidine nucleotide is substituted for a purine nucleotide (e.g., G to T, or C to G). A nucleotide substitution within a coding sequence that results in the substitution of an amino acid also can be referred to as a non-synonymous SNP.

In some embodiments, a Brassica plant can include all or part of the genomic sequence on chromosome N1 between nucleotide positions 8879780 and 11922690 that confers higher PUFA content. In some embodiments, the genomic sequence that confers higher PUFA content can include one or more SNPs (e.g., two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more different SNPs) between nucleotide positions 8879780 and 11922690 on chromosome N1. Table 9 provides examples of SNPs within chromosome N1 that are distributed throughout the genomic sequence between nucleotide positions 8879780 and 11922690, including SNPs at positions 8,952,616, 9,040,901, 9,046,609, 9,048,617, 9,136,686, 9,143,608, 9,248,592, 9,347,120, 9,352,326, 9,454,361, 9,549,523, 9,641,936, 9,652,028, 9,794,198, 9,847,417, 9,921,975, 9,952,792, 10,052,015, 10,402,684, 10,425,211, 10,558,464, 10,613,015, 10,659,284, 10,706,805, 10,748,492, 10,852,010, 11,007,740, 11,047,958, 11,150,929, 11,269,217, 11,343,118, 11,455,979, 11,565,970, 11,659,776, 11,726,807, and 11,850,103. Table 10 provides examples of SNPs in candidate genes (e.g., genes that encode products involved in lipid biosynthesis or a related pathway in the parent which increases PUFA) within chromosome N1 between nucleotide positions 8879780 and 11922690 including positions 9,136,686, 9,641,936, 10,613,015, 9,040,901, 9,048,617, 9,352,326, 9,921,975, and 10,706,805. In some embodiments, all or part of the genomic sequence on chromosome N1 between nucleotide positions 8879780 and 11922690 that confers higher PUFA content can include one or more non-synonymous SNPs at positions 9,136,686, 9,143,608, 9,454,361, 9,952,792, 9,549,523 9,641,936, 9,652,028, 10,613,015, 9,352,326, 9,794,198, 9,847,417, 9,921,975, 10,402,684, 10,706,805, 10,659,284, 10,748,492, 11,007,740, 11,047,958, 11,150,929, 11,269,217, 11,455,979, 11,659,776, or 11,850,103.

In some embodiments, a Brassica plant can include all or part of the genomic sequence on chromosome N1 between nucleotide positions 22,823,086 and 24,045,492 that confers higher PUFA content. The genomic sequence associated with higher PUFA content can include one or more SNPs (e.g., two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more different SNPs) between nucleotide positions nucleotide positions 22,823,086 and 24,045,492 on chromosome N1. Table 11 provides examples of SNPs within chromosome N1 that are distributed throughout the genomic sequence between nucleotide positions 22,823,086 and 24,045,492, including SNPs at positions 22,823,086, 22,880,595, 22,902,670, 22,949,738, 23,011,207, 23,044,228, 23,099,592, 23,176,771, 23,201,595, 23,257,618, 23,302,268, 23,367,822, 23,380,089, 23,457,696, 23,520,607, 23,552,773, 23,598,941, 23,670,623, 23,682,848, 23,745,365, 23,792,572, 23,855,829, 23,910,029, 23,947,522, and 24,021,883. Table 12 provides examples of SNPs in candidate genes (e.g., genes that encode products involved in lipid biosynthesis or a related pathway in the parent which increases PUFA) within chromosome N1 between nucleotide positions 22,823,086 and 24,045,492 including positions 23,089,542, 23,089,635, 23,090,743, 23,090,785, 23,091,367, 23,092,042, 23,150,402, 23,150,595, 23,155,220, 23,155,766, 23,314,197, 23,318,357, 23,343,089, 23,679,276, 23,679,287, 23,679,396, 23,886,929, 23,925,895, 23,963,309, 24,029,270, 24,029,279, and 24,029,294. In some embodiments, all or part of the genomic sequence on chromosome N1 between nucleotide positions 22,823,086 and 24,045,492 that confers higher PUFA content can include one or more non-synonymous SNPs at positions 23,089,542, 23,089,635, 23,090,743, 23,090,785, 23,091,367, 23,092,042, 23,099,592, 23,150,402, 23,150,595, 23,155,220, 23,155,766, 23,201,595, 23,257,618, 23,314,197, 23,318,357, 23,380,089, 23,457,696, 23,520,607, 23,552,773, 23,598,941, 23,679,276, 23,679,287, 23,679,396, 23,682,848, 23,745,365, 23,855,829, 23,925,895, 23,947,522, 24,021,883, 24,029,270, 24,029,279, or 24,029,294.

In some embodiments, a Brassica plant can include all or part of the genomic sequence on chromosome N6 between nucleotide positions 19,156,645 and 20,846,412 that confers higher PUFA content. The genomic sequence that confers higher PUFA content can include one or more SNPs (e.g., two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more different SNPs) between nucleotide positions nucleotide positions 19,156,645 and 20,846,412 on chromosome N6. Table 13 provides examples of SNPs within chromosome N6 that are distributed throughout the genomic sequence between nucleotide positions 19,156,645 and 20,846,412, including SNPs at positions 19,156,645, 19,199,109, 19,325,186, 19,402,086, 19,513,420, 19,583,431, 19,601,021, 19,706,563, 19,800,643, 19,906,666, 20,000,119, 20,095,002, 20,205,211, 20,300,571, 20,406,148, 20,407,023, 20,505,840, 20,601,198, 20,631,917, and 20,702,631. Table 14 provides examples of SNPs in candidate genes (e.g., genes that encode products involved in lipid biosynthesis or a related pathway in the parent which increases PUFA) within chromosome N6 between nucleotide positions 19,156,645 and 20,846,412 including positions 19,336,744, 19,336,819, 19,337,615, 19,350,156, 19,353,584, 19,353,648, 19,353,749, 19,476,836, 19,783,834, 19,784,007, 19,784,367, 19,784,633, 19,784,672, 19,784,688, 19,784,733, 19,800,525, 20,191,826, 20,300,548, 20,375,643, 20,766,637, 20,769,461, 20,770,769, 20,823,998, 20,825,959, 20,826,301, 20,827,570, 20,827,573, and 20,912,356. In some embodiments, all or part of the genomic sequence on chromosome N6 between nucleotide positions 19,156,645 and 20,846,412 that confers higher PUFA content can include one or more non-synonymous SNPs at positions 19,325,186, 19,336,744, 19,336,819, 19,337,615, 19,350,156, 19,353,584, 19,353,648, 19,353,749, 19,402,086, 19,513,420, 19,783,834, 19,784,007, 19,784,367, 19,784,633, 19,784,672, 19,784,688, 19,784,733, 19,800,525, 19,906,666, 20,000,119, 20,095,002, 20,300,548, 20,375,643, 20,766,637, 20,769,461, 20,770,769, 20,823,998, 20,825,959, 20,826,301, 20,827,570, or 20,827,573.

In some embodiments, a Brassica plant can include all or part of the genomic sequence on chromosome N1 between nucleotide positions 8879780 and 11922690 that confers higher PUFA content and all or part of the genomic sequence on chromosome N1 between nucleotide positions 22,823,086 and 24,045,492 that confers higher PUFA content. Examples of SNPs that can be found in each of these regions are described above.

In some embodiments, a Brassica plant can include all or part of the genomic sequence on chromosome N1 between nucleotide positions 8879780 and 11922690 that confers higher PUFA content and all or part of the genomic sequence on chromosome N6 between nucleotide positions 19,156,645 and 20,846,412 that confers higher PUFA content. Examples of SNPs that can be found in each of these regions are described above.

In some embodiments, a Brassica plant can include all or part of the genomic sequence on chromosome N1 between nucleotide positions 22,823,086 and 24,045,492 that confers higher PUFA content and can include all or part of the genomic sequence on chromosome N6 between nucleotide positions 19,156,645 and 20,846,412 that confers higher PUFA content. Examples of SNPs that can be found in each of these regions are described above.

In some embodiments, the Brassica plants provided herein (e.g., Brassica napus plants) produce seeds with an EPA content of at least about 6%, at least about 7%, at least about 8%, at least about 9%, at least about 10%, at least about 11%, at least about 12%, at least about 13%, at least about 15%, at least about 16%, or at least about 17% based on total weight of fatty acids (C14-C22). In some embodiments, the EPA content can range from at least about 6% to about 18% (e.g., about 8% to about 18%, about 10% to about 18%, about 12% to about 18%, about 12.5% to about 17.5%, or about 12.5% to about 15%).

In some embodiments, the Brassica plants provided herein (e.g., Brassica napus plants) produce seeds with a DHA content of at least about 0.9%, at least about 1.0%, at least about 1.2%, at least about 1.3%, at least about 1.4%, at least about 1.5%, at least about 1.6%, at least about 1.7%, at least about 1.8%, at least about 1.9%, or at least about 2% based on total weight of fatty acids (C14-C22). In some embodiments, the DHA content can range from about 0.9% to about 2% (e.g., about 0.9% to about 1.5%, about 1.0% to about 2.0%, about 1.0% to about 1.9%, or about 1.2% to about 1.9%).

In some embodiments, the Brassica plants provided herein (e.g., Brassica napus plants) produce seeds with a DPA content of at least about 3.5%, at least about 4.0%, at least about 4.5%, at least about 5.0%, at least about 5.5%, or at least about 6% based on total weight of fatty acids (C14-C22). In some embodiments, the DPA content can range from about 3.5% to about 6%, or from about 4.0% to about 6% (e.g., about 4% to about 5%, about 4.75% to about 6.0%, about 4.75% to about 5.75%, or about 5.0% to about 6.0%).

In some embodiments, the Brassica plants provided herein (e.g., Brassica napus plants) produce seeds with a DHA content of about 0.5% to about 2.8% and/or an EPA content of about 3.5% to about 15.0% EPA. In some embodiments, the DHA content can range from about 0.9 to about 1.5% and/or an EPA content of about 12.5% to about 15.0%.

In some embodiments, the Brassica plants provided herein (e.g., Brassica napus plants) produce seeds with an EPA, DPA, and DHA content of at least about 17%, at least about 18%, at least about 19%, at least about 20%, at least about 21%, at least about 22%, or at least about 23%. In some embodiments, the Brassica plants produce seeds with an EPA, DPA, and DHA content that ranges from about 19% to about 24% (e.g., about 20% to about 24%, about 20% to about 23%, or about 21% to about 23%).

Brassica plants described herein that produce seeds having higher levels of EPA, DPA, and/or DHA compared to corresponding control plants (e.g., plants lacking the T-DNA expression construct and/or lacking the genomic sequence(s) from chromosome N1 and/or chromosome N6 that confers higher PUFA content), and the parts thereof, can be used for feed purposes such as aquaculture feed, e.g. as described in AU2011289381A and members of the patent family thereof.

In some embodiments, a Brassica plant provided herein is tolerant of an herbicide such as an imidazolinone, dicamba, cyclohexanedione, a sulfonylurea, glyphosate, glufosinate, phenoxy propionic acid, L-phosphinothricin, a triazolinone, a triazolpyrimidine, a pyrimidinylthiobenzoate, and benzonitrile. For example, Brassica plants can include a polynucleotide that encodes a product (e.g., a mutant acetohydroxyacid synthase) that confers resistance to an herbicide (e.g., an imidazolinones, a sulfonylureas, a pyrimidinylthiobenzoate, a triazolinone, or a triazolopyrimidine). See, for example, Tans et al., Pest Manag Sci. 61(3):246-57 (2005) and Hu et al., PLoS One. 12(9): e0184917 (2017).

The present disclosure also relates to oil comprising a polyunsaturated fatty acid obtainable from the plants described herein. The term “oil” refers to a fatty acid mixture comprising unsaturated and/or saturated fatty acids which are esterified to triglycerides. In some embodiments, the triglycerides in the oil of the disclosure comprise PUFA or VLC-PUFA moieties as referred to above. The amount of esterified PUFA and/or VLC-PUFA is, in some embodiments, approximately 30%, or at least 50%, or at least 60%, 70%, 80% or more. The oil may further comprise free fatty acids, such as the PUFA and VLC-PUFA referred to above.

The oils according to the disclosure can have an EPA content of at least about 6%, at least about 7%, at least about 8%, at least about 9%, at least about 10%, at least about 11%, at least about 12%, at least about 13%, at least about 15%, at least about 16%, or at least about 17% based on the total fatty acid content. The oils according to the disclosure can have a DHA content of at least about 0.9%, at least about 1.0%, at least about 1.2%, at least about 1.3%, at least about 1.4%, at least about 1.5%, at least about 1.6%, at least about 1.7%, at least about 1.8%, at least about 1.9%, or at least about 2% based on the total fatty acid content. In some embodiments, the oils of the disclosure can have a DHA content of about 0.5 to about 2.8% and/or EPA content of about 3.5% to about 15.0% EPA. In some embodiments, an oil of the disclosure can have a DHA content of about 0.9 to about 1.5% and/or an EPA content of about 12.5% to about 15.0% EPA. In some embodiments, an oil of the disclosure can have an EPA, DPA, and DHA content of at least about 15%, at least about 16%, at least about 17%, at least about 18%, at least about 19%, at least about 20%, at least about 21%, at least about 22%, or at least about 23%. In some embodiments, an oil of the disclosure can have an EPA, DPA, and DHA content that ranges from about 19% to about 24% (e.g., about 20% to about 24%, about 20% to about 23%, or about 21% to about 23%).

In some embodiments, the plants, such as the progeny, can be hybrids or inbreds. The term hybrid relates to a cultivar or plant-breeding progeny based upon the controlled cross-pollination between or among distinct parent lines, so that the resulting seed inherits its genetic composition from those parent lines. Seed for a particular hybrid can be repeatedly and predictably produced when repeatedly making controlled cross-pollinations from the same stable female and male parent genotypes. While inbred refers to a relatively stable plant genotype resulting from doubled haploids, successive generations of controlled self-pollination, successive generations of controlled backcrossing to a recurrent parent, or other method to develop homozygosity. Backcrossing refers to a process in which a breeder repeatedly crosses hybrid progeny back to one of the parents; for example, a first-generation hybrid F1 crossed back to one of the parental genotypes of the F1 hybrid. The production of hybrid plants is well known/available to an art worker.

The invention will be further described in the following examples, which do not limit the scope of the invention described in the claims.

### Examples

### Example 1: Genome Mapping to Identify Quantitative Trait Loci (QTL) that Increase EPA, DPA and/or DHA

### Materials and Methods

### Plant Propagation

Brassica plants were grown in a growth chamber (Conviron GR192) in Berger B7 soil with 100 ppm fertilizer at every watering (Jack's 20-20-20) from rosette stage through end of flowering. Chamber conditions were 16-hour day length with 22° C. day temperature and 19° C. night temperature, or at 28° C. day temperature and 15° C. night temperature. Seed was harvested at full maturity.

### Cross Pollination

Homozygous PUFA donor LBFLFK (LBFLFK bears two insertions of the VC-LTM593-1qcz plasmid at two different loci; LBFLFK and the genetic elements of VC-LTM593-1qcz rc and the function of each element are provided in PCT/EP2015/076632 (published as WO/2016/075327) was used as pollen donor in a cross with many unique Brassica accessions to make F1 seed. The F1 seed fatty acid profile was determined by gas chromatography.

### Genotypic Analysis

Leaf samples were taken from each accession prior to flowering. DNA was extracted from each leaf sample using DNeasy minipreps (Qiagen) and was analyzed for single nucleotide polymorphisms (SNPs) by Illumina Infinium 60k Brassica array and KASP (competitive allele-specific polymerase chain reaction (PCR), LGC).

### Phenotypic Analysis

Fatty acid profile of seed was measured by gas chromatography using ˜30 seeds and standard fatty acid methyl ester preparation (adapted from AOCS method Ce 1-62) immediately following crushing. GLC-566 (NuChek Prep) was used for the standard and fatty acid profiles were determined by ChemStation software (Agilent) as a percent of total fatty acids.

The fatty acid composition of seeds was determined by a modification of American Oil Chemist's Society (AOCS) protocol Ce 1-62. In the procedure fatty acids present as acylglycerols are converted to fatty acid methyl esters, which are analyzed by gas liquid chromatography (GLC or GC). For each sample to be analyzed 20-30 seeds are placed in a 15 ml centrifuge tube along with two steel ball bearings. The tube is capped and shaken for 30 seconds or until the seeds are visibly crushed. Approximately 0.6 mL of 2 N KOH in methanol is added to the tube, and the tube is shaken again for approximately one minute. The tube and its contents are placed in a water bath at 70±5° C. for 2 min. After removing the tube from the bath 4 mL of water saturated with sodium chloride and 2.0 mL of isooctane with 100 ppm of BHT are added, the tube is shaken and centrifuged for 1 min. in a tabletop centrifuge. A portion of the isooctane supernatant is transferred to a gas chromatographic (GC) vial and capped. Vials are stored at 0-4° C. until analysis, but no more than five days.

Fatty acid methyl esters were subject to analysis on a GC on an instrument equipped with a 20 m×0.18 mm×0.2 μm DB-225 (50% Cyanopropylphenyl) column from Agilent Technologies. An injector temperature of 250° C. was applied and 1 μl was injected with a split of 50:1 using 0.8 ml/min Hydrogen column flow (constant flow mode). Initial temperature is 190° C./0 min->15 C°/min->220° C.->220° C./9 min. and a flame ionization detector. The instrument is calibrated with a fatty acid methyl ester standard, such as NuChek Prep Catalog number GLC 566.

The content of fatty acids having from 14 carbon atoms (C14 fatty acids) to 24 carbon atoms (C24 fatty acids) is determined using the integrated peak area for each type of fatty acid reported normalized to the total peak area for those fatty acids.

The levels of particular acids are provided herein in percentages. Unless specifically noted otherwise, such percentages are weight percentages based on the total fatty acids in the seed oil, as calculated experimentally. Thus, for example, if a percentage of a specific species of fatty acid is provided, e.g., oleic acid, this is a w/w percentage based on the total fatty acids detected in the seed oil.

### Genetic Mapping

### QTL (Quantitative Trait Loci) Mapping

A high-density single nucleotide polymorphism (SNP) Illumina Infinium array containing 52,157 markers (Clarke et al. 2016) was used in this study to genotype a total of 288 Brassica accessions. DNA was isolated, quantified and hybridized to the array as described in the manufacturer's protocol (Illumina Inc., San Diego, CA). The arrays were scanned using an Illumina HiScan or BeadArray Reader, and SNP data were analyzed using the Genotyping module of the GenomeStudio software package. A total of 47,304 SNPs passed quality analysis and were used to perform the GWAS (Genome-Wide Association Study) analysis in the R environment (R Development Core Team, 2015). The GAPIT package, a genomic association and prediction integrated tool (Version 2; Lipka et al. 2012) and the GWAS function in the rrBLUP package (Endelman, 2011) were both used to identify genomic blocks conferring the PUFA phenotype including EPA, DPA and DHA content. Oil trait distribution and correlation analyses were also carried out in an R environment.

### QTL Validation

Two backcrossing (BC₁) populations were developed from crosses between the PUFA donor line, Kumily LBFLFK, and two Brassica accessions carrying favorable alleles for EPA and DHA based on the association mapping results. Selections were genotyped to confirm copy number of the PUFA events. LBFLFK contains two PUFA loci (e.g., two insertions of the construct carrying the PUFA pathway). Lines that were homozygous for both loci or heterozygous for both loci were chosen for mapping. A total of 658 BC₁ lines were selected from both populations (Table 1).

TABLE 1
Number of homozygous and heterozygous lines selected from each
BC1 population and used for QTL mapping.

| Line | # Selected |
|---|---|
| 2 | 127 homozygous 2 loci, 130 heterozygous |
| | 2 loci |
| 4 | 174 homozygous 2 loci, 227 heterozygous |
| | 2 loci |

DNA from these 658 BC1 lines was genotyped with 1434 genome-wide Ion AmpliSeq sequencing (Life Technologies) SNP markers along with 85 KASP (LGC, UK) SNP markers located within the QTL regions identified from GWAS analysis. A subset of SNPs was polymorphic for each line.

Correlations between fatty acid composition and single SNP markers were calculated in Microsoft Excel (2010). Linkage maps of the experimental populations were constructed using the Kosambi function of JoinMap 3.0 (Kyazma). Quantitative trait loci (QTL) mapping was done in the R/qtl program of the R statistical package (Broman et al., 2003; Broman and Sen, 2009).

### Results

### Genome-Wide Association Study Mapping

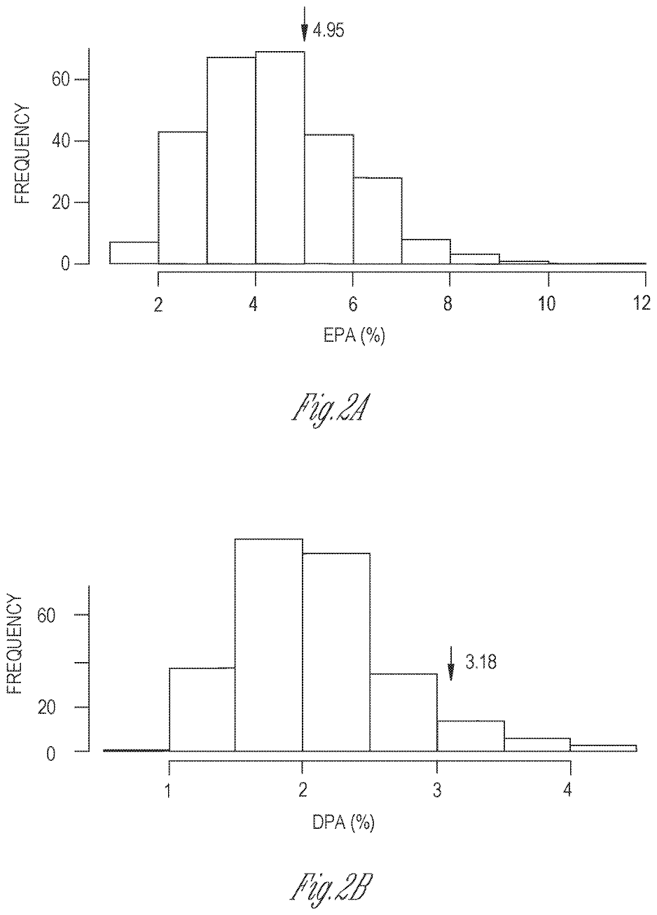

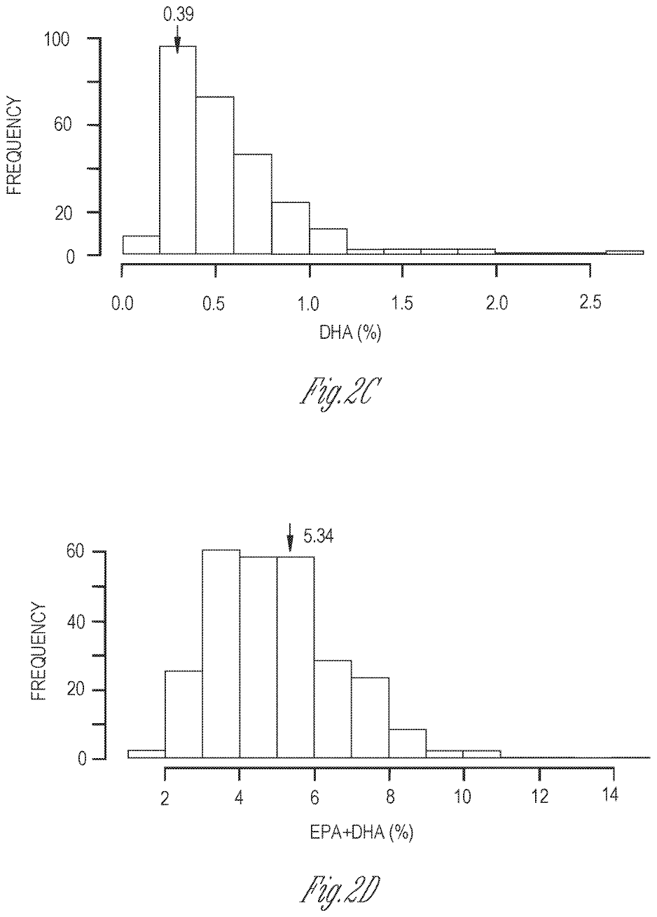

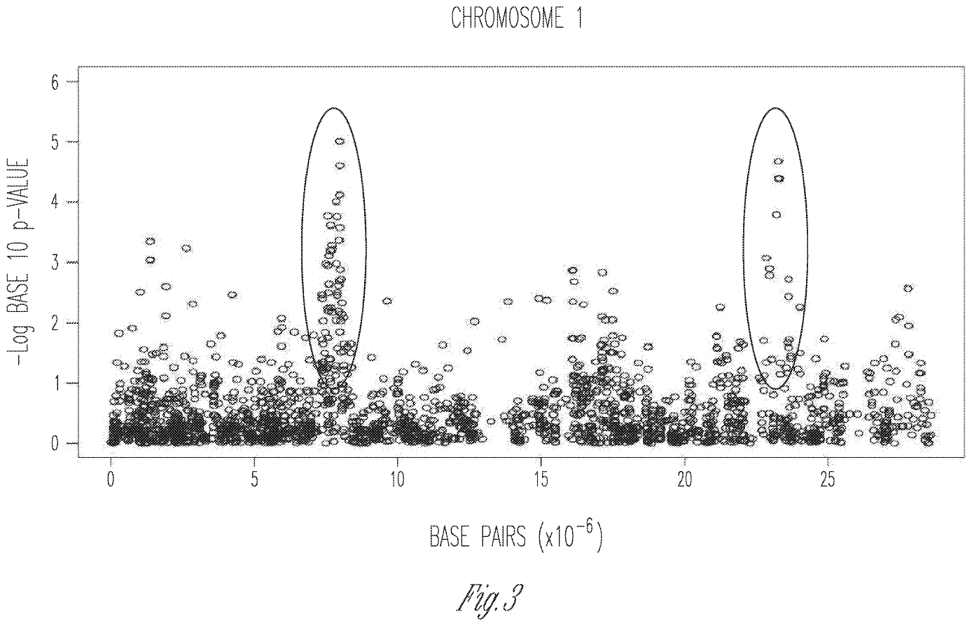

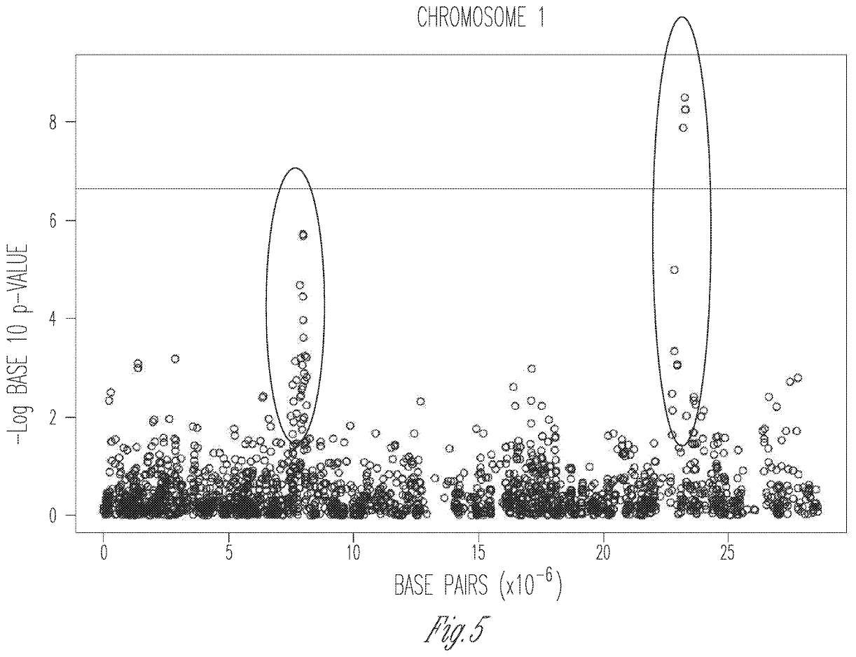

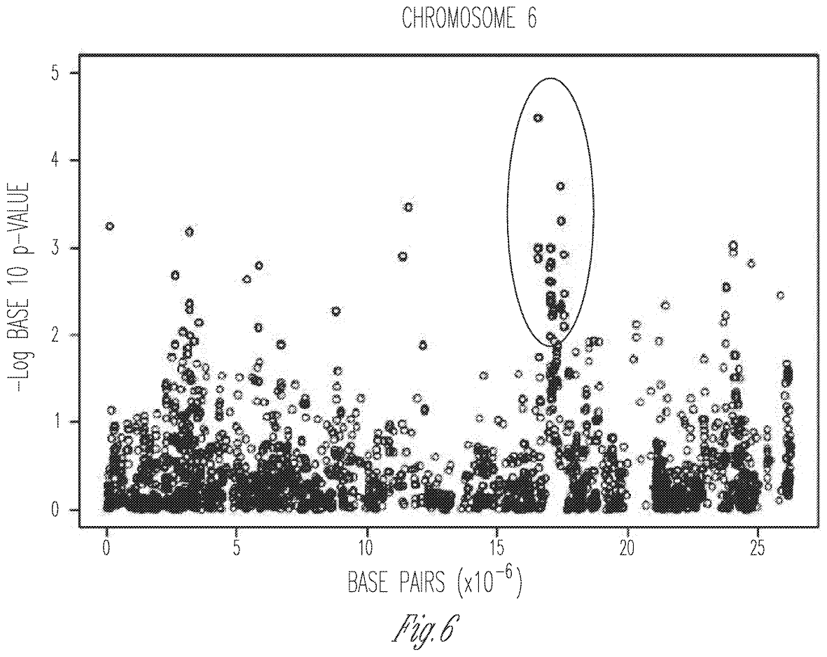

FIG. 2 shows the distribution of EPA, DPA and DHA from 279 Brassica accessions heterozygous for each LBFLFK insertion (arrow shows the average content from the PUFA donor line, Kumily LBFLFK). Genome wide association study analyses identified two significant associations on the A01 chromosome (FIGS. 3, 4 and 5) and one significant association on A06 (FIG. 6). Specific SNP markers for the QTL are specified in Tables 3, 4, and 5. Haplotype analysis identified haplotypes correlated with PUFA traits, including two favorable haplotypes corresponding to the two QTLs on A01, respectively, which increase the content of EPA, DPA and DHA. For example, accessions carrying the favorable haplotype corresponding to QTL1 had an average 55% increase in EPA (6.70% compared to 4.32%), 101% increase in DPA (4.16% compared to 2.07%), and 121% increase in DHA (1.17% compared to 0.53%). Lines carrying the favorable haplotype for the QTL on A05 had an average 71% increase in EPA (7.28% compared to 4.26%), 54% increase in DPA (3.10% compared to 2.01%), and 163% increase in DHA (1.34% compared to 0.51%). And finally, lines carrying the favorable haplotype for the QTL on A06 had an average 53% increase in EPA (6.79% compared to 4.43%), 79% increase in DPA (3.70% compared to 2.07%), and 162% increase in DHA (1.44% compared to 0.55%).

### QTL Validation

QTL scans of the BC1 population confirmed the presence of the loci identified in the GWAS study (QTL1, 2, and 3; Table 6). Here it can be seen that individuals heterozygous at each of the specified loci contained higher PUFA contents than individuals homozygous for the PUFA donor line, Kumily LBFLFK.

TABLE 2
Summary of EPA + DHA values of Brassica napus lines
carrying various combinations of QTL on A01.
“+” indicates the presence of the favorable (PUFA
increasing) genotype and “−” indicates the alternative allele.

| Line | QTL1-A01 | QTL2-A01 | EPA + DHA |
|---|---|---|---|
| Brassica rapa 1* | + | + | 14.7 |
| Brassica napus 2 | + | − | 12.1 |
| Brassica napus 3 | + | + | 11.6 |
| Brassica napus 4 | + | + | 10.8 |
| Brassica napus 5 | + | + | 10.7 |
| Brassica napus 6 | − | + | 10.4 |
| Brassica napus 7 | + | − | 9.8 |
| Brassica napus 8 | − | − | 9.4 |
| Brassica napus 9* | + | − | 9.3 |
| Brassica napus 10 | + | − | 9 |

TABLE 3
SNP locations of the first genomic block (QTL1) on NO1

| SNP markers | Chromosome | Position in DH12075 (V3.0) |
|---|---|---|
| Bn-A01-p22949106 | 1 | 22452876 |
| Bn-A01-p23195492 | 1 | 22672365 |
| Bn-A01-p23253678 | 1 | 22730061 |
| Bn-A01-p23259034 | 1 | 22735485 |
| Bn-A01-p23614494 | 1 | 23132864 |

TABLE 4
SNP locations of the 2^{nd} genomic block (QTL2) on N01

| SNP markers | Chromosome | Position in DH12075 (V3.0) |
|---|---|---|
| Bn-A01-p7961620 | 1 | 8008195 |
| Bn-A01-p7973418 | 1 | 8025818 |
| Bn-A01-p7974551 | 1 | 8027772 |
| Bn-A01-p7979458 | 1 | 8033912 |
| Bn-A01-p7983241 | 1 | 8037687 |
| Bn-A01-p7987687 | 1 | 8044316 |

TABLE 5
SNP location of the 3^{rd} genomic block (QTL3) on N06

| SNP markers | Chromosome | Position in DH12075 (3.0) |
|---|---|---|
| Bn-A06-p8634648 | 6 | 8317333 |
| Bn-A06-p8697187 | 6 | 8381641 |
| Bn-A06-p8697193 | 6 | 8381647 |
| Bn-A06-p8732612 | 6 | 8395518 |
| Bn-A06-p8766088 | 6 | 8429433 |
| Bn-A06-p14664213 | 6 | 16607904 |
| Bn-A06-p14809503 | 6 | 16742528 |
| Bn-A06-p14813811 | 6 | 16746780 |

TABLE 6
Markers and average PUFA values for alternate genotypes at each locus.

| | | Position in | | | | | EPA + |
|---|---|---|---|---|---|---|---|
| | | DH12075 | | | | | DPA + |
| | SNP Marker | (v3.0) | Genotype | C20:5 | C22:5 | C22:6 | DHA |
| QTL1 | Bn-A01-p14943499 | 14441849 | CC | 6.092 | 2.852 | 0.531 | 9.475 |
| | | | CT | 6.364 | 2.986 | 0.581 | 9.932 |
| | Bn-A01-p21393260 | 21381690 | GG | 5.955 | 2.815 | 0.517 | 9.287 |
| | | | GA | 7.106 | 3.242 | 0.639 | 10.988 |
| | 23_199_N1_G277T | 23302241 | GG | 5.993 | 2.835 | 0.519 | 9.347 |
| | | | TG | 6.533 | 3.027 | 0.602 | 10.162 |
| | Bn-A01-p26447438 | 24568625 | TT | 6.008 | 2.853 | 0.516 | 9.378 |
| | | | GT | 6.479 | 2.984 | 0.596 | 10.059 |
| QTL2 | Bn-A01-p7479483 | 7515195 | CC | 6.049 | 2.818 | 0.55 | 9.417 |
| | | | CA | 6.374 | 2.987 | 0.561 | 9.922 |
| | Bn-A01-p9614730 | 9123417 | GC | 6.052 | 2.833 | 0.535 | 9.421 |
| | | | GA | 6.411 | 2.992 | 0.577 | 9.98 |
| | Bn-A01-p9961553 | 9496833 | CC | 6.028 | 2.825 | 0.533 | 9.386 |
| | | | CT | 6.4 | 2.986 | 0.576 | 9.962 |
| | Bn-A01-p14943499 | 14441849 | CC | 6.092 | 2.852 | 0.531 | 9.475 |
| | | | CT | 6.364 | 2.986 | 0.581 | 9.932 |
| QTL3 | Bn-A06-p7499751 | 7197412 | TT | 5.548 | 2.608 | 0.494 | 8.649 |
| | | | GT | 6.208 | 2.821 | 0.565 | 9.594 |
| | Bn-A06-p7670696 | 7381938 | AA | 5.501 | 2.591 | 0.493 | 8.585 |
| | | | GA | 6.218 | 2.821 | 0.562 | 9.6 |
| | Bn-A06-p19185585 | 14926457 | TT | 5.573 | 2.607 | 0.499 | 8.679 |
| | | | CT | 6.146 | 2.803 | 0.555 | 9.504 |
| | Bn-A06-p19333055 | 15086022 | GG | 5.539 | 2.59 | 0.499 | 8.628 |
| | | | AG | 6.128 | 2.801 | 0.551 | 9.479 |
| | Bn-A06-p19562187 | 15302019 | TT | 5.568 | 2.61 | 0.498 | 8.676 |
| | | | GT | 6.145 | 2.8 | 0.556 | 9.501 |
| | Bn-A06-p14813811 | 16746780 | GG | 5.597 | 2.614 | 0.502 | 8.713 |
| | | | TG | 6.204 | 2.825 | 0.563 | 9.592 |
| | Bn-A06-p15712062 | 17641645 | GG | 5.524 | 2.571 | 0.499 | 8.594 |
| | | | TG | 6.174 | 2.826 | 0.557 | 9.558 |
| | Bn-A06-p16015727 | 17961288 | GG | 5.566 | 2.609 | 0.49 | 8.664 |
| | | | AG | 6.384 | 2.947 | 0.578 | 9.908 |
| | Bn-A06-p17114458 | 18994122 | GG | 5.528 | 2.585 | 0.49 | 8.602 |
| | | | AG | 6.187 | 2.823 | 0.565 | 9.574 |
| | Bn-A06-p18500596 | 20405390 | AA | 5.52 | 2.613 | 0.48 | 8.613 |
| | | | GA | 6.237 | 2.813 | 0.576 | 9.626 |
| | Bn-A06-p21490506 | 20769890 | TT | 5.565 | 2.626 | 0.479 | 8.669 |
| | | | CT | 6.197 | 2.801 | 0.578 | 9.576 |

Subsequent fine mapping analysis was performed to validate and narrow the QTL regions for QTL1, 2 and 3 to the genomic regions that are identified in Table 7. Fine mapping was performed by crossing the Parent 1 non-PUFA/Parent 2 PUFA F₁ with the elite female parent, backcrossing twice to elite female parent containing LBFLFK, selfing and using the BC₂S₂ and BC₂S₃ selfed populations to map. In each of these generations, selections were made using correlation of SNP genotype with VLC-PUFA as described in QTL Mapping.

Simultaneously, the QTL markers identified in mapping and fine mapping as described herein were used to introgress the QTL into elite parent lines, including the elite female parent shown in Table 8. For introgression, the elite parent line homozygous for LBFLFK (“Control” in Table 8) was crossed to Parent 2 which contained both LBFLFK and QTL1 and QTL2 and crossed to Parent 3 which contained both LBFLFK and QTL3. The resultant progeny were selected for the respective QTL and backcrossed twice to the elite parent line. The resultant progeny from each cross were then crossed to each other to combine LBFLFK, QTL1, QTL2 and QTL3 in a single plant. The progeny were selfed and plants homozygous for LBFLFK, QTL1, QTL2 and QTL3 were selected, selfed and were grown in chambers (28° C. day temperature and 15° C. night temperature), and the fatty acid profile was assessed in seeds harvested from the plants. As shown in Table 8, plants with QTL3 had an average 15% increase in overall PUFA content (18.07% compared to 15.67%), with an average 12% increase in EPA (12.66% compared to 11.32%), average 19% increase in DPA (4.26% compared to 3.57%), and average 46% increase in DHA (1.15% compared to 0.79%). Plants with both QTL1 and QTL2 had an average 10% increase in overall PUFA content (17.3% compared to 15.67%), with an average 12% increase in EPA (12.73% compared to 11.32%), average 2% increase in DPA (3.64% compared to 3.57%), and average 19% increase in DHA (0.94% compared to 0.79%). Plants with QTL1, QTL2, and QTL3 had an average 28% increase in overall PUFA content (20.04% compared to 15.67%), with an average 27% increase in EPA (14.32% compared to 11.32%), average 24% increase in DPA (4.41% compared to 3.57%), and average 65% increase in DHA (1.30% compared to 0.79%).

Plants homozygous for LBFLFK, QTL1, QTL2 and QTL3 were also field grown. The test plots contained three selections of a female parent containing the three QTL which have been described to confer the increase in EPA, DPA and DHA. Two control plots were included, which are of the same parental background but do not carry any of the three QTL. The field results confirm the results from the growth chamber (discussed above), where the addition of the three described genomic regions confer an increase in EPA, DPA and DHA in the field. The control plots averaged 9.44% EPA+DPA+DHA, while the plots with the three described QTL averaged 16.17% EPA+DPA+DHA, and the highest plot contained 17.74% EPA+DPA+DHA. These three QTL have demonstrated an increase in EPA+DPA+DHA of up to 87.9% over the control, with an average 58% increase in EPA (11.08% compared to 7.03%), average 83% increase in DPA (3.47% compared to 1.90%), and average 65% increase in DHA (0.90%6 compared to 0.34%). All fatty acid analyses were performed by GC as described, using approximately 30 seeds subsampled from the full plot sample.

TABLE 7
Markers identifying the refined genomic regions for
QTL1, 2 and 3.

| | Size | | QTL Position in |
|---|---|---|---|
| QTL | (MB) | SNP Boundaries | DH12075 |
| QTL1: N1.1 | 3 | Bn-A01-p9326695, | 8879780-11922690 |
| | | Bn-A01-p12510328 | |
| QTL2: N1.2 | 1.2 | Bn-A01-p23330944, | 22823086-24045492 |
| | | Bn-A01-p24835449 | |
| QTL3: N6 | 1.7 | Bn-A06-p17291868, | 19156645-20846412 |
| | | Bn-A06-p21415201 | |

TABLE 8
Average PUFA values for Brassica plants having the indicated QTL

| QTL | | EPA + DPA + DHA | EPA | DPA | DHA |
|---|---|---|---|---|---|
| N1.1, | avg | 20.04 | 14.32 | 4.41 | 1.30 |
| N1.2, | stdev | 1.74 | 1.34 | 0.51 | 0.26 |
| N6 | n | 17 | 17 | 17 | 17 |
| | max | 22.95 | 16.74 | 5.45 | 1.87 |
| N1.1, | avg | 17.30 | 12.73 | 3.64 | 0.93 |
| N1.2 | stdev | 2.23 | 1.65 | 0.52 | 0.18 |
| | n | 20 | 20 | 20 | 20 |
| | max | 21.96 | 16.05 | 4.48 | 1.42 |
| N6 | avg | 18.07 | 12.66 | 4.26 | 1.15 |
| | stdev | 2.40 | 1.95 | 0.42 | 0.22 |
| | n | 17 | 17 | 17 | 17 |
| | max | 20.88 | 15.26 | 4.87 | 1.45 |
| Control | avg | 15.67 | 11.32 | 3.57 | 0.79 |
| | stdev | 0.89 | 0.51 | 0.52 | 0.10 |
| | n | 5 | 5 | 5 | 5 |
| | max | 16.96 | 11.88 | 4.41 | 0.93 |

The region corresponding to each QTL was sequenced. The genomic sequence for each QTL was compared between the parent line with favourable alleles for increasing PUFA and the PUFA donor line, Kumily LBFLFK. Table 9 provides SNPs in chromosome N1 that were distributed across the QTL1 interval and Table 10 provides SNPs in chromosome N1 that are in candidate genes within QTL1. Table 11 provides SNPs in chromosome N1 that were distributed across the QTL2 interval and Table 12 provides SNPs in chromosome N1 that are in candidate genes within QTL2. Table 13 provides SNPs in chromosome N6 that were distributed across the QTL3 interval and Table 14 provides SNPs in chromosome N6 that are in candidate genes within QTL3. In each of these tables, “ref nt” refers to reference nucleotide in the PUFA donor line; “alt nt” refers to alternate nucleotide in the line with favourable alleles for increased PUFA; “AA change” refers to amino acid change; “type” refers to the polymorphism type; “'S” refers to transition; “TV” refers to transversion; and “subst” refers to substitution.

TABLE 9
SNPs in chromosome N1 distributed across the QTL1 interval

| Position | Ref Nt | Alt Nt | Sequence | Codon Change | AA Change | Type | Affected gene | Ortholog | Description |
|---|---|---|---|---|---|---|---|---|---|
| 8,750,203 | T | C | ATTTGAGAGTGGGAAA | TCT −> CCT | S −> P | TS | maker- | AT4G26170.1 | \| Symbols: \| FUNCTIONS IN: molecular |
| | | | AGACTTTGTAGGTAGTA | | | | N1- | | function unknown; INVOLVED IN: |
| | | | GATACAGAGTCCAAGA | | | | augustus- | | biological process unknown; LOCATED |
| | | | A[T/C]CTATCCAGGGTC | | | | gene- | | IN: chloroplast; EXPRESSED IN: 7 plant |
| | | | TTGGTGTAGCTGTCAAC | | | | 8.369 | | structures; EXPRESSED DURING: F |
| | | | ATTCATGACGCAGATGA | | | | | | mature embryo stage, petal |
| | | | CATT (SEQ ID NO: 1) | | | | | | differentiation and expansion stage, E |
| | | | | | | | | | expanded cotyledon stage, D bilateral |
| | | | | | | | | | stage; BEST Arabidopsis thaliana protein |
| | | | | | | | | | match is: effector of transcription2 |
| | | | | | | | | | (TAIR: AT5G56780.1); Has 75 Blast hits to |
| | | | | | | | | | 42 proteins in 17 species: Archae-0; |
| | | | | | | | | | Bacteria-2; Metazoa-2; Fungi-0; |
| | | | | | | | | | Plants-70; Viruses-0; Other |
| | | | | | | | | | Eukaryotes-1 (source: NCBI BLink). \| |
| | | | | | | | | | chr4: 13256930-13259382 FORWARD |
| | | | | | | | | | LENGTH = 2453 |
| 8,844,910 | C | T | CTGATACATTTAATAGA | CCG −> CCA | None | TS | maker- | AT4G26400.1 | \| Symbols: \| RING/U-box superfamily |
| | | | TTACCGTAAAACAAGTT | | | | N1- | | protein \| chr4: 13344808-13346597 |
| | | | TGCTGTACACCAGCTT[ | | | | augustus- | | REVERSE LENGTH = 1790 |
| | | | [C/T]GGAGGTGCTGAGAG | | | | gene- | | |
| | | | CACTTCTGCTTCTGCAG | | | | 8.434 | | |
| | | | AAACGGGAAGATAAAC | | | | | | |
| | | | AAC (SEQ ID NO: 2) | | | | | | |
| 8,952,616 | G | A | TTTGTCGTCAACGTCGC | None | None | TS | maker- | AT4G26590.1 | \| Symbols: ATOPT5, OPT5 \| oligopeptide |
| | | | CTTCTTCCACATCCCCCA | | | | N1- | | transporter 5 \| chr4: 13413973- |
| | | | TATAAACCTTAAACA[G/A] | | | | augustus- | | 13417055 REVERSE LENGTH = 3083 |
| | | | CAACAAGAGATTTTT | | | | gene- | | |
| | | | AATGGAAACTGATGAA | | | | 8.442 | | |
| | | | GACTGATCTAATAGTCA | | | | | | |
| | | | TA (SEQ ID NO: 3) | | | | | | |
| 9,046,609 | G | A | TCATTGCTAATGCTTTT | CAC −> CAT | None | TS | maker- | AT4G26760.1 | \| Symbols: MAP65-2 \| microtubule- |
| | | | GTTTTGAACACCATTTG | | | | N1- | | associated protein 65-2 \| |
| | | | CCTCATCTAAGCTCGG | | | | fgenesh- | | chr4: 13478592-13481808 REVERSE |
| | | | [G/A]TGAACTTCAGTGA | | | | gene- | | LENGTH = 3217 |
| | | | CCGTGGTCAAGAAATCT | | | | 9.391 | | |
| | | | AAACCAAGGACGGCGC | | | | | | |
| | | | ATAG (SEQ ID NO: 4) | | | | | | |
| 9,143,608 | G | A | AGTTGGACCTGAGCTTA | TGC −> TAC | C −> Y | TS | maker- | #N/A | #N/A |
| | | | GTTTCCTTAAAGACGTG | | | | N1- | | |
| | | | GGACGGGCTGAGTACT | | | | fgenesh- | | |
| | | | [G/A]CAGGGTTACCGAT | | | | gene- | | |
| | | | GAAGAAGCCTTAGAAG | | | | 9.335 | | |
| | | | GTGCTCTACTACTTACTT | | | | | | |
| | | | GGC (SEQ ID NO: 5) | | | | | | |
| 9,248,592 | C | T | ACATATCTTATTGGTTA | None | None | TS | None | None | None |
| | | | TACATGATCCTCGTTTCT | | | | | | |
| | | | GTCATCTTCATACTC | | | | | | |
| | | | [C/T]GAAACAAAAAAATTA | | | | | | |
| | | | AAAATCACCATATTAAT | | | | | | |
| | | | TGCAAAGTTGTATCAAT | | | | | | |
| | | | T (SEQ ID NO: 6) | | | | | | |
| 9,347,120 | G | A | CCGTTGAGAATCGTCCT | GAC −> GAT | None | TS | augustus_ | AT4G27410.2 | \| Symbols: RD26, ANAC072 \| NAC (No |
| | | | GAGCGAATTCATCCGA | | | | masked | | Apical Meristem) domain transcriptional |
| | | | GGAAGATCAAAAGACC | | | | -N1- | | regulator superfamily protein \| |
| | | | G[G/A]TCTTTCATCTCC | | | | abinit- | | chr4: 13707240-13709149 REVERSE |
| | | | GGGAACGAATCAAGAA | | | | gene- | | LENGTH = 1910 |
| | | | CGTCGTCGAGCTGAGA | | | | 9.68 | | |
| | | | CGAAGA (SEQ ID NO: 7) | | | | | | |
| 9,454,361 | C | T | CTCCCATCTGTCACTGC | GCA −> ACA | A −> T | TS | maker- | AT4G27540.1 | \| Symbols: PRA1.H \| prenylated RAB |
| | | | AGTACTTGAACAGCTCC | | | | N1- | | acceptor 1.H \| chr4: 13753210- |
| | | | CAAAGTGCTAAACTGG | | | | fgenesh- | | 13754745 REVERSE LENGTH = 1536 |
| | | | [C/T]TAACAACCCCACCA | | | | gene- | | |
| | | | ATGCAAGTGGCATCTG | | | | 9.420 | | |
| | | | ATACCTACAAAGAAACA | | | | | | |
| | | | CCA (SEQ ID NO: 8) | | | | | | |
| 9,549,523 | C | T | AACCATATCTACTCGTA | GAT −> AAT | D −> N | TS | augustus | AT4G27654.1 | \| Symbols: \| unknown protein; |
| | | | GGTTGGTCTTGATCGAT | | | | _masked | | FUNCTIONS IN: molecular function |
| | | | TCTAGTTGAGAAGTAT | | | | -N1- | | unknown; INVOLVED IN: biological |
| | | | [C/T]GATCGGTGCCAGTT | | | | abinit- | | process unknown; LOCATED IN: |
| | | | CACAAGTGTCGATCGAT | | | | gene- | | endomembrane system; EXPRESSED IN: |
| | | | GATGTGCGTCTTTTTTT | | | | 9.111 | | 17 plant structures; EXPRESSED |
| | | | GC (SEQ ID NO: 9) | | | | | | DURING: 9 growth stages; Has 30201 |
| | | | | | | | | | Blast hits to 17322 proteins in 780 |
| | | | | | | | | | species: Archae-12; Bacteria-1396; |
| | | | | | | | | | Metazoa-17338; Fungi-3422; Plants- |
| | | | | | | | | | 5037; Viruses-0; Other Eukaryotes- |
| | | | | | | | | | 2996 (source: NCBI BLink). \| |
| | | | | | | | | | chr4: 13811645-13812126 FORWARD |
| | | | | | | | | | LENGTH = 482 |
| 9,652,028 | T | G | TTCGCAGTGGTTCCGGT | GAT −> GAG | D −> E | TV | augustus_ | AT4G27750.1 | \| Symbols: ISI1 \| binding \| |
| | | | GCTTTTTCGACATTCGTT | | | | masked- | | chr4: 13841688-13843633 FORWARD |
| | | | GAAAGAAGGGGAGGA | | | | N1- | | LENGTH = 1946 |
| | | | [T/G]GATAGAGTGACGA | | | | abinit- | | |
| | | | GCTTGGATCACATATTC | | | | gene- | | |
| | | | AGCGTTGAGCCGATGA | | | | 9.134 | | |
| | | | AGAT (SEQ ID NO: 10) | | | | | | |
| 9,794,198 | C | T | ACCAAACTCTGGATACG | GCT −> GTT | A −> V | TS | maker- | AT4G28162.1 | \| Symbols: \| Potential natural antisense |
| | | | AAGCTTCTGGTCCAAGA | | | | N1- | | gene, locus overlaps with AT4G28160 \| |
| | | | TCCAGAGAAGATCAAG | | | | augustus- | | chr4: 13980327-13980820 REVERSE |
| | | | [C/T]TCCACCAGTGCCAC | | | | gene- | | LENGTH = 494 |
| | | | AACCATGACTTGAAAAC | | | | 9.249 | | |
| | | | AGTCAACAAATAAGCGT | | | | | | |
| | | | TT (SEQ ID NO: 11) | | | | | | |
| 9,847,417 | A | G | ATTTCGATAGAGTCGCT | ATT −> GTT | I −> V | TS | augustus_ | AT4G28310.1 | \| Symbols: \| unknown protein; BEST |
| | | | GGAGAGAGCGACTCCA | | | | masked- | | Arabidopsis thaliana protein match is: |
| | | | AAGCCATTAAGAAACC | | | | N1- | | unknown protein (TAIR: AT1G52270.1); |
| | | | G[A/G]TTGAATCAAAGT | | | | abinit- | | Has 30201 Blast hits to 17322 proteins |
| | | | CCATCGTGAAGGATAA | | | | gene- | | in 780 species: Archae-12; Bacteria- |
| | | | GAAGAAGAAGACTGAG | | | | 9.179 | | 1396; Metazoa-17338; Fungi-3422; |
| | | | TCAAGC (SEQ ID NO: | | | | | | Plants-5037; Viruses-0; Other |
| | | | 12) | | | | | | Eukaryotes-2996 (source: NCBI BLink). |
| | | | | | | | | | \| chr4: 14017408-14018208 FORWARD |
| | | | | | | | | | LENGTH = 801 |
| 9,952,792 | G | A | TGGGTAATCTCAGCCAC | GCC −> ACC | A −> T | TS | maker- | AT5G47390.1 | \| Symbols: \| myb-like transcription |
| | | | TACTCGGGCTCTGGGTT | | | | N1- | | factor family protein \| chr5: 19226790- |
| | | | GAGCGGGCTTGGCGGA | | | | augustus- | | 19228858 FORWARD LENGTH = 2069 |
| | | | [G/A]CCGGGTCGAACA | | | | gene- | | |
| | | | ATCCTGGTTCTCCCGGT | | | | 9.258 | | |
| | | | GATGGCCATGACCACG | | | | | | |
| | | | GCGTC (SEQ ID NO: 13) | | | | | | |
| 10,052,015 | A | C | ATAGTCAACAGCCGTCT | GTA −> GTC | None | TV | augustus_ | AT2G31290.2 | \| Symbols: \| Ubiquitin carboxyl-terminal |
| | | | CAAGAACATCATCCTAA | | | | masked- | | hydrolase family protein \| |
| | | | GGTGTCCCAACCAGGT | | | | N1- | | chr2: 13343846-13346070 FORWARD |
| | | | [A/C]GCACCCGTTAAGTT | | | | abinit- | | LENGTH = 2225 |
| | | | TCTACAGAAGAAGTTCA | | | | gene- | | |
| | | | AGACTCTTGATCTCCAA | | | | 10.331 | | |
| | | | GG (SEQ ID NO: 14) | | | | | | |
| 10,402,684 | C | A | TGCATAAGTACTACTCG | GGG −> TGG | G −> W | TV | maker- | AT4G16660.1 | \| Symbols: \| heat shock protein 70 (Hsp |
| | | | AACCCATGTCATAAAAT | | | | N1- | | 70) family protein \| chr4: 9376737- |
| | | | ATAACATGTCTCGACC | | | | augustus- | | 9381507 FORWARD LENGTH = 4771 |
| | | | [C/A]ATTAGAGAAATCCT | | | | gene- | | |
| | | | TATCAATCCCATACTGC | | | | 10.157 | | |
| | | | AAAGCCGCACCGGAAT | | | | | | |
| | | | GCT (SEQ ID NO: 15) | | | | | | |
| 10,425,211 | A | T | GAGCTCGAGTTCCTCAA | GCA −> GCT | None | TV | augustus_ | AT2G47330.1 | \| Symbols: \| P-loop containing |
| | | | CCACGAACTAAACAGTA | | | | masked- | | nucleoside triphosphate hydrolases |
| | | | GCGGGGAAGACAAAGC | | | | N1- | | superfamily protein \| chr2: 19428897- |
| | | | [A/T]GAAGAACTGTAAA | | | | abinit- | | 19431720 REVERSE LENGTH = 2824 |
| | | | GGTCAAGCAGAAGCTG | | | | gene- | | |
| | | | AAGAAGACGAAGATGA | | | | 10.401 | | |
| | | | GAAGCC (SEQ ID NO: 16) | | | | | | |
| 10,558,464 | T | C | AAGTTTGGTGTTGACCT | CAT −> CAC | None | TS | maker- | AT4G16470.1 | \| Symbols: \| Tetratricopeptide repeat |
| | | | GTTAACATGTTCTAGCT | | | | N1- | | (TPR)-like superfamily protein |
| | | | TTCAGGTAGAGCATCA | | | | fgenesh- | | chr4: 9287862-9289582 REVERSE |
| | | | [T/C]AGAAAGAAAGAGC | | | | gene- | | LENGTH = 1721 |
| | | | AGCCTGACAAGACACTA | | | | 10.31 | | |
| | | | CAAGGTCTTTGCATCAC | | | | | | |
| | | | TGG (SEQ ID NO: 17) | | | | | | |
| 10,659,284 | A | G | TCACAACCTAAGTCATA | ATT −> GTT | I −> V | TS | augustus_ | AT4G16350.1 | \| Symbols: CBL6, SCABP2 \| calcineurin |
| | | | TTCATATTACCAATTGC | | | | masked- | | B-like protein 6 \| chr4: 9242320- |
| | | | AGTTACTGTGAATGAG | | | | N1- | | 9243912 REVERSE LENGTH = 1593 |
| | | | [A/G]TTGAAGCTCTTTAC | | | | abinit- | | |
| | | | GAAATGTTCAAGAGCAT | | | | gene- | | |
| | | | CAGCAAAGACGGCCTT | | | | 10.449 | | |
| | | | ATC (SEQ ID NO: 18) | | | | | | |
| 10,748,492 | G | A | GACTATGCTGCTTTCGA | CCT −> TCT | P −> S | TS | fgenesh_ | AT3G22980.1 | \| Symbols: \| Ribosomal protein |
| | | | GAGTAAGAGCTTCACT | | | | masked- | | S5/Elongation factor G/III/V family |
| | | | GTACAATGCTGAGTCA | | | | N1- | | protein \| chr3: 8160156-8163316 |
| | | | G[G/A]TGGTGTCTCAGT | | | | abinit- | | REVERSE LENGTH = 3161 |
| | | | ATGATCTTCTGTGAAGC | | | | gene- | | |
| | | | CAAGTCTGTGAGAGAC | | | | 10.205 | | |
| | | | ATGAG (SEQ ID NO: 19) | | | | | | |
| 10,852,010 | G | A | TCACCAAACTCACATGT | TTC −> TTT | None | TS | augustus_ | #N/A | #N/A |
| | | | CTGTTTCTTGACGTCTCT | | | | masked- | | |
| | | | TGCACACACTTTGTA[G/A] | | | | N1- | | |
| | | | AAGATTGCAATAATA | | | | abinit- | | |
| | | | TTAAGCTTTCCTTGTTCC | | | | gene- | | |
| | | | ACACGTTTCTTCATCAT | | | | 10.476 | | |
| | | | (SEQ ID NO: 20) | | | | | | |
| 11,007,740 | G | A | TTCCATCAGGAGGTTCT | GGT −> AGT | G −> S | TS | augustus_ | AT4G15810.1 | \| Symbols: \| P-loop containing |
| | | | GATGATTTTATGGTGCA | | | | masked- | | nucleoside triphosphate hydrolases |
| | | | AAATGAGGGGAGTATA | | | | N1- | | superfamily protein \| chr4: 8989162- |
| | | | [G/A]GTTCTAAGCAACT | | | | abinit- | | 8992591 REVERSE LENGTH = 3430 |
| | | | CATCGAGGAGGATGGG | | | | gene- | | |
| | | | GGTGGTAGCATTCAAG | | | | 11.275 | | |
| | | | AATCT (SEQ ID NO: 21) | | | | | | |
| 11,047,958 | G | C | TTATCTCTAGCAAGTTA | AAG −> ACC | K −> N | TV | maker- | #N/A | #N/A |
| | | | CAATGAGATATTGGAT | | | | N1- | | |
| | | | GGAAGTGTTGATACTA | | | | augustus- | | |
| | | | A[G/C]TGTCTTATTGGT | | | | gene- | | |
| | | | ATATAATTITGAACTGC | | | | 11.77 | | |
| | | | TTCTCTTAGATTATTTTT | | | | | | |
| | | | ATC (SEQ ID NO: 22) | | | | | | |
| 11,150,929 | G | A | CTCTCCTCGCTTGGTGA | CCG −> TCG | P −> S | TS | maker- | AT4G15560.1 | \| Symbols: CLA1, DEF, CLA, DXS, DXPS2 \| |
| | | | AACCGGAGAGACCATT | | | | N1- | | Deoxyxylulose-5-phosphate synthase \| |
| | | | GGTTTGCCTCATTGTCG | | | | augustus- | | chr4: 8883907-8887565 FORWARD |
| | | | [G/A]CATCTTTCCTCTTCT | | | | gene- | | LENGTH = 3659 |
| | | | ACCGGTAAGAATCTTGT | | | | 11.116 | | |
| | | | GAGGATAAGACTGATG | | | | | | |
| | | | AC (SEQ ID NO: 23) | | | | | | |
| 11,269,217 | G | A | CCTGTCTCTAACATCTG | CGT −> TGT | R −> C | TS | augustus | #N/A | #N/A |
| | | | CTGCAGAGTTACCCGCT | | | | masked | | |
| | | | TCCACCATATCTCCAC | | | | -N1- | | |
| | | | [G/A]GAGCCTGTACGCGA | | | | abinit- | | |
| | | | TTAGGCGAAGAGGACT | | | | | | |
| | | | ATCTCTTGGTTCTTTCTT | | | | gene- | | |
| | | | GA (SEQ ID NO: 24) | | | | 11.325 | | |
| 11,343,118 | G | C | TCCGAAACCTTGACCAA | GTG −> GTC | None | TV | augustus_ | AT4G15390.1 | \| Symbols: \| HXXXD-type acyl- |
| | | | ATTCTACCCCCTCGCGG | | | | masked- | | transferase family protein \| |
| | | | GAAGAATCAACGGAGT | | | | N1- | | chr4: 8792812-8794293 REVERSE |
| | | | [G/C]ACTGTCGACTGTA | | | | abinit- | | LENGTH = 1482 |
| | | | ACGACGAAGGAGCTGT | | | | gene- | | |
| | | | TTTCGTCGACGCTCGTG | | | | 11.333 | | |
| | | | TCGA (SEQ ID NO: 25) | | | | | | |
| 11,455,979 | A | T | GGCTTGGTCTGGTAGG | TGT −> AGT | C −> S | TV | maker- | AT4G15060.1 | \| Symbols: \| CONTAINS InterPro |
| | | | CATAATGAAAGCCGCTT | | | | N1- | | DOMAIN/s: FBD (InterPro: IPR013596), F- |
| | | | GACATGTGTGATTAAAC | | | | fgenesh- | | box domain, Skp2-like |
| | | | [A/T]AACAAAGTTCTGT | | | | gene- | | (InterPro: IPR022364), FBD-like |
| | | | ATGTCAAGGTATGTCGA | | | | 11.54 | | (InterPro: IPR006566), Leucine-rich |
| | | | ATCGACTTCCATCTCCTC | | | | | | repeat 2 (InterPro: IPR013101); BEST |
| | | | CA (SEQ ID NO: 26) | | | | | | Arabidopsis thaliana protein match is: |
| | | | | | | | | | FBD, F-box and Leucine Rich Repeat |
| | | | | | | | | | domains containing protein |
| | | | | | | | | | (TAIR: AT1G55660.1); Has 30201 Blast |
| | | | | | | | | | hits to 17322 proteins in 780 species: |
| | | | | | | | | | Archae-12; Bacteria-1396; Metazoa- |
| | | | | | | | | | 17338; Fungi-3422; Plants-5037; |
| | | | | | | | | | Viruses-0; Other Eukaryotes-2996 |
| | | | | | | | | | (source: NCBI BLink). \| chr4: 8599035- |
| | | | | | | | | | 8601761 FORWARD LENGTH = 2727 |
| 11,565,970 | C | T | AAGAGCGATGGGCTTG | TCC −> TCT | None | TS | fgenesh_ | AT4G14920.1 | \| Symbols: \| Acyl-CoA N-acyltransferase |
| | | | GTGACTTTTCTTCCTTTC | | | | masked- | | with RING/FYVE/PHD-type zinc finger |
| | | | CGGAGACGAGTGATTC | | | | N1- | | protein \| chr4: 8531043-8535842 |
| | | | [C/T]GAGTCTAGCGATA | | | | abinit- | | REVERSE LENGTH = 4800 |
| | | | AGCCCCTCATGGCACAC | | | | gene- | | |
| | | | TATGGCAATGTAGAAG | | | | 11.201 | | |
| | | | AGCC (SEQ ID NO: 27) | | | | | | |
| 11,659,776 | C | G | GATCTGTCGAGCTTAGT | AAG −> AAC | K −> N | TV | maker- | AT4G14750.1 | \| Symbols: IQD19 \| IQ-domain 19 \| |
| | | | CGCTATCCACGGATAAT | | | | N1- | | chr4: 8470381-8472187 FORWARD |
| | | | ACTCATAATGACTTTC | | | | augustus- | | LENGTH = 1807 |
| | | | [C/G]TTTCCTAATTGAGAT | | | | gene- | | |
| | | | GAAGCTCTATGCACCCG | | | | 11.142 | | |
| | | | TACGGCCTTAAGTACAC | | | | | | |
| | | | C (SEQ ID NO: 28) | | | | | | |
| 11,726,807 | C | T | GTGTATATCGTTAACCC | CTG −> CTA | None | TS | maker- | AT4G14730.1 | \| Symbols: \| Bax inhibitor-1 family |
| | | | CACGACCATTGTAGCTG | | | | N1- | | protein \| chr4: 8448549-8450073 |
| | | | TGAGAACCGCAGCCTC | | | | augustus- | | FORWARD LENGTH = 1525 |
| | | | [C/T]AGAACAATCTTCCC | | | | gene- | | |
| | | | TGTTTTTTTTATTCACAA | | | | 11.144 | | |
| | | | GAGTTAGATTGTAACAT | | | | | | |
| | | | C (SEQ ID NO: 29) | | | | | | |
| 11,850,103 | A | G | AAGCACTCGACTTATAA | CTG −> CCG | L −> P | TS | augustus_ | AT4G25000.1 | \| Symbols: ATAMY1, AMY1 \| alpha- |
| | | | CCATGTTTGATATGTTT | | | | masked- | | amylase-like \| chr4: 12851969-12853845 |
| | | | CGACGATCTACCAGTC | | | | N1- | | REVERSE LENGTH = 1877 |
| | | | [CA/G]GCTTCTGTGAAGT | | | | abinit- | | |
| | | | CTGCTCAGCAACGTGGT | | | | gene- | | |
| | | | CAGCCCTTTCATGACCA | | | | 11.410 | | |
| | | | CTC (SEQ ID NO: 30) | | | | | | |
| 11,956,477 | C | T | CACCATCACCAGCTATG | CCA −> TCA | P −> S | TS | augustus_ | AT4G14580.1 | \| Symbols: CIPK4, SnRK3.3 \| CBL- |
| | | | GGCTTTCCATCTCCACC | | | | masked- | | interacting protein kinase 4 \| |
| | | | ATCACCAGAAAAAGCC[ | | | | N1- | | chr: 8367887-8369167 REVERSE |
| | | | [C/T]CAGGGACCATTCTG | | | | | | LENGTH = 1281 |
| | | | CTCGGTAAATACGAACT | | | | | | |
| | | | CGGTCGCCGATTAGGC | | | | | | |
| | | | AGT (SEQ ID NO: 31) | | | | | | |

TABLE 10
SNPs in chromosome N1 that are in candidate genes within QTL1

| | | | | | Amino | | | | |
|---|---|---|---|---|---|---|---|---|---|
| | Ref | Alt | | Codon | Acid | | Affected | | |
| Position | Nt | Nt | Sequence | Change | Change | Type | gene | Ortholog | Description |
| 9,136,686 | A | C | TAGTTGAGAA | AAG -> | K -> Q | TV | augustus_ | AT4G27030.1 | \| Symbols: |
| | | | ACGTTTTGTG | CAG | | | masked-N1- | | FAD4, FADA \| |
| | | | AGTCCTCCTC | | | | abinit- | | fatty acid |
| | | | TTTCCAACGA | | | | gene-9.32 | | desaturase A \| |
| | | | CCCAACTCTG | | | | | | chr4: 13571921- |
| | | | [A/C]AGTCT | | | | | | 13573070 FORWARD |
| | | | ACATGGACTC | | | | | | LENGTH = 1150 |
| | | | ACCGCTTATG | | | | | | |
| | | | GGTTGCAGCT | | | | | | |
| | | | GGTTGCACCA | | | | | | |
| | | | CCTTG (SEQ | | | | | | |
| | | | ID NO: 32) | | | | | | |
| 9,641,936 | G | A | TATTGATAAG | CCT -> | P -> L | TS | maker-N1- | AT5G42870.2 | \| Symbols: |
| | | | TATGGATTCG | CTT | | | augustus- | | PAH2 \| |
| | | | AGTACCAATT | | | | gene-9.307 | | phosphatidic acid |
| | | | CAATGGTAAA | | | | | | phosphohydrolase 2 \| |
| | | | AAAGATGTCA | | | | | | chr5: |
| | | | [G/A]GTCTT | | | | | | 17185478- |
| | | | ACCTGCTTTA | | | | | | 17189943 |
| | | | GATTATTTAA | | | | | | REVERSE |
| | | | GAAACTTCTT | | | | | | LENGTH = 4466 |
| | | | GTTAGATATG | | | | | | |
| | | | CCTGA (SEQ | | | | | | |
| | | | ID NO: 33) | | | | | | |
| 10,613,015 | C | G | TTTAAACAGG | CAG | Q -> E | TV | maker-N1- | AT4G16440.1 | \| Symbols: \| |
| | | | TTGCTTGAAC | GAG | | | augustus- | | ferredoxin |
| | | | GGTGGTGGGC | | | | gene-10.123 | | hydrogenases |
| | | | AGATTAAGCC | | | | | | \| chr4: |
| | | | AAAAACGGGA | | | | | | 9269094- |
| | | | [C/G]AGACT | | | | | | 9271669 |
| | | | CCGAAAGAAC | | | | | | REVERSE |
| | | | TGATCAACTC | | | | | | LENGTH = 2576 |
| | | | ACTTGAAGCT | | | | | | |
| | | | ACTTATATGA | | | | | | |
| | | | ATGAT (SEQ | | | | | | |
| | | | ID NO: 34) | | | | | | |
| 9,040,901 | A | G | AGGAGGATGA | AAT | None | TS | maker-N1- | AT5G55240.1 | \| Symbols: |
| | | | GAATTCGTGT | -> | | | fgenesh- | | ATPXG2 \| |
| | | | TACCGGAAGA | AAC | | | gene-9.389 | | ARABIDOPSIS |
| | | | GTAGCATAGC | | | | | | THALIANA |
| | | | TAAGGGCCAA | | | | | | PEROXYGENASE 2 \| |
| | | | [A/G]TTGAT | | | | | | chr5: |
| | | | TACAGCGGCT | | | | | | 22405926- |
| | | | GCGATAAGCG | | | | | | 22407351 |
| | | | ATACAATGAT | | | | | | FORWARD |
| | | | ATTGAAACCA | | | | | | LENGTH = 1426 |
| | | | AGCAT (SEQ | | | | | | |
| | | | ID NO: 35) | | | | | | |
| 9,048,617 | C | A | TATATTAATC | None | None | TV | maker-N1- | AT4G26770.1 | \| Symbols: \| |
| | | | CAGGACATTG | | | | augustus- | | Phosphatidate |
| | | | GCATTGTAAG | | | | gene-9.206 | | cytidylyltransferase |
| | | | AAAAATCATT | | | | | | family protein \| |
| | | | TTCAAAAGGG | | | | | | chr4: |
| | | | [C/A]AATGG | | | | | | 13482074- |
| | | | AGAAAGATCT | | | | | | 13484849 |
| | | | GAATCAGAAC | | | | | | FORWARD |
| | | | TCTCCACGAA | | | | | | LENGTH = 2776 |
| | | | TCAGAAAGCT | | | | | | |
| | | | AAGGG (SEQ | | | | | | |
| | | | ID NO: 36) | | | | | | |
| 9,352,326 | A | G | AGCAAATACA | TTT | F -> S | TS | augustus_ | AT4G27420.1 | \| Symbols: \| |
| | | | TATTATATTT | -> | | | masked-N1- | | ABC-2 type |
| | | | ATATATTAAA | TCT | | | abinit- | | transporter |
| | | | ATACCTGATC | | | | gene-9.69 | | family protein \| |
| | | | TTGTAAGTGA | | | | | | chr4: |
| | | | [A/G]AAAGG | | | | | | 13712199- |
| | | | TCCGTTTTCC | | | | | | 13714797 |
| | | | ACCAAAGGAG | | | | | | REVERSE |
| | | | ACCACATAAG | | | | | | LENGTH = 2599 |
| | | | AAAGACACGA | | | | | | |
| | | | CAAAA | | | | | | |
| | | | (SEQ ID | | | | | | |
| | | | NO: 37) | | | | | | |
| 9,921,975 | T | GA | GGATATGATT | ATG | M -> R | TV | maker-N1- | AT4G17480.1 | \| Symbols: \| |
| | G | | GAGACAGGGA | -> | | | augustus- | | alpha/beta- |
| | | | TTAATGATCA | AGA | | | gene-9.257 | | Hydrolases |
| | | | ATGCTCAAGC | | | | | | superfamily |
| | | | GTTGAAGCTA | | | | | | protein \| |
| | | | [TG/GA]AGC | | | | | | chr4: 9745006- |
| | | | TTCACACAGC | | | | | | 9746966 |
| | | | TCCTTAGGAA | | | | | | REVERSE |
| | | | CCTCTCCAGC | | | | | | LENGTH = 1961 |
| | | | TCCCCTGGCT | | | | | | |
| | | | ACTGCTT | | | | | | |
| | | | (SEQ ID | | | | | | |
| | | | NO: 38) | | | | | | |
| 10,706,805 | G | T | AAAGCAATCG | GAC | D -> E | TV | maker-N1- | AT1G54350.1 | \| Symbols: \| |
| | | | ACTCAGCATT | -> | | | augustus- | | ABC transporter |
| | | | TTCCCTGACG | GAA | | | gene-10.169 | | family |
| | | | CGGACAAGGT | | | | | | protein \| |
| | | | TGTAACGAAA | | | | | | chr1: |
| | | | [G/T]TCAGC | | | | | | 20286882-20290401 |
| | | | CTCTTTCTTC | | | | | | FORWARD |
| | | | TCTTGCATAA | | | | | | LENGTH = 3520 |
| | | | AGTTGAGATT | | | | | | |
| | | | CACTAGTCCC | | | | | | |
| | | | TGAGA (SEQ | | | | | | |
| | | | ID NO: 39) | | | | | | |

TABLE 11
SNPs in chromosome N1 distributed across the QTL2 interval

| | Ref | Alt | | Codon | AA | | Affected | | |
|---|---|---|---|---|---|---|---|---|---|
| Position | Nt | Nt | Sequence | Change | Change | Type | gene | Ortholog | Description |
| 22,823,086 | T | C | CACCATTTCTAGATC | None | None | TS | maker- | AT3G1700 | \| Symbols: |
| | | | TATAGAATCAGAATT | | | | N1- | 0.1 | UBC32 \| ubiquitin- |
| | | | CGAATTGGATCTTCG | | | | augustus | | conjugating |
| | | | GTGAA[T/C]CTAATC | | | | -gene- | | enzyme 32 \| |
| | | | TCCAATATCATCTCC | | | | 22.349 | | chr3: |
| | | | TCTGATCTCTAAAGC | | | | | | 5797179- |
| | | | TTGAGGAGGATAAC | | | | | | 5799683 |
| | | | (SEQ ID NO: | | | | | | RD |
| | | | 40) | | | | | | LENGTH = 2505 |
| 22,880,595 | A | C | ATAGAGTAACGATT | GTT -> | None | TV | maker- | AT3G1683 | \| Symbols: |
| | | | ATCATCAACCTTAGT | GTG | | | N1- | 0.1 | TPR2 \| TOPLESS- |
| | | | GAACCCTGAGAGAT | | | | fgenesh- | | related 2 \| |
| | | | ACTTCTC[A/C]ACTT | | | | gene- | | chr3: |
| | | | CGTCCCATTCACCGG | | | | 22.246 | | 5731534- |
| | | | CGAGAGCTTTCTCTT | | | | | | 5737772 |
| | | | CAAAGTACTTTATAT | | | | | | RD |
| | | | T (SEQ ID NO: | | | | | | LENGTH = 6239 |
| | | | 41) | | | | | | |
| 22,902,670 | G | A | ATGTTTATTCATTTTT | None | None | TS | None | None | None |
| | | | CATAAGTTTTTTTTA | | | | | | |
| | | | GTTTTCATTTACATTT | | | | | | |
| | | | TAT[G/A]TATGATTT | | | | | | |
| | | | TATCAAACTACATAA | | | | | | |
| | | | ATAAAAAATCTACAA | | | | | | |
| | | | ACTTTAATCAAA | | | | | | |
| | | | (SEQ ID NO: | | | | | | |
| | | | 42) | | | | | | |
| 22,949,738 | C | T | CATTATCCTGAAGCA | ACG -> | None | TS | maker- | AT3G1671 | \| Symbols: |
| | | | ATGAGTTTCTAACCT | ACA | | | N1- | 0.1 | \| Pentatricopeptide |
| | | | GTAAATATACTTATA | | | | augustus | | repeat (PPR) |
| | | | ACCTG[C/T]GTGTCC | | | | -gene- | | superfamily |
| | | | TCTCCCCCAAAACTG | | | | 22.357 | | protein \| |
| | | | CAACTGAGCCATCAT | | | | | | chr3: |
| | | | ATGCTTGAGATCAA | | | | | | 5690020- |
| | | | (SEQ ID NO: | | | | | | 5691543 |
| | | | 43) | | | | | | RD |
| | | | | | | | | | LENGTH = 1524 |
| 23,011,207 | C | T | TCCTCCCACATAGGT | GTC -> | None | TS | maker- | AT3G1650 | \| Symbols: |
| | | | CAAATCAATAAGAA | GTT | | | N1- | 0.1 | PAP1, IAA26 \| |
| | | | TGGTGATGGTGTGA | | | | augustus | | phytochrome- |
| | | | AGCAAGT[C/T]GAA | | | | -gene- | | associated |
| | | | CCTAAGAGGGAAGGC | | | | 23.126 | | protein 1 \| |
| | | | ATGTTTGTAAAGATC | | | | | | chr3: |
| | | | AACATGGACAGTGT | | | | | | 5612500- |
| | | | TCC (SEQ ID NO: | | | | | | 5614410 REVERSE |
| | | | 44) | | | | | | LENGTH = 1911 |
| 23,044,228 | C | G | CTATATTTTATAACA | None | None | TV | None | None | None |
| | | | ACAACTTTCGCAATA | | | | | | |
| | | | TCATATTTTTATGGA | | | | | | |
| | | | TTAAT[C/G]ATGTGG | | | | | | |
| | | | TAATAAAAATTCTAG | | | | | | |
| | | | TTCTCCCAAATTAAG | | | | | | |
| | | | GTACAAAATTAATG | | | | | | |
| | | | (SEQ ID NO: | | | | | | |
| | | | 45) | | | | | | |
| 23,099,592 | A | G | CAGCAAGCCAGCCT | ATG -> | M -> V | TS | maker- | AT3G1625 | \| Symbols: |
| | | | CCAGTCGCAGATGA | GTG | | | N1- | 0.1 | NDF4 \| NDH |
| | | | GCCGAATGATGAAC | | | | augustus | | -dependent |
| | | | CTCCTGCT[A/G]TGG | | | | -gene- | | cyclic electron |
| | | | ATTTTGCGTTCGTCC | | | | 23.129 | | flow 1 \| |
| | | | ATGTAAGTAGTGAT | | | | | | chr3: |
| | | | CTTCACTCGCTAGTT | | | | | | 5506931- |
| | | | GCT (SEQ ID NO: | | | | | | 5508414 REVERSE |
| | | | 46) | | | | | | LENGTH = 1484 |
| 23,176,771 | A | G | CTCACAGAGCTATAC | TTT -> | None | TS | maker- | AT3G1609 | \| Symbols: |
| | | | ATAAACAAACCATCC | TTC | | | N1- | 0.1 | \| RING/U-box |
| | | | ACAACAAGAAGAAA | | | | fgenesh- | | superfamily |
| | | | CGCCAT[A/G]AACG | | | | gene- | | protein \| |
| | | | AAACAATACGAACG | | | | 23.75 | | chr3: |
| | | | TGCGCGAGCTTAGA | | | | | | 5456144- |
| | | | GACGTTAGGAGTCG | | | | | | 5458966 |
| | | | TCTC (SEQ ID NO: | | | | | | FORWARD |
| | | | 47) | | | | | | LENGTH = 2823 |
| 23,201,595 | G | A | TGATAAGGTTACTGC | AGT -> | S -> N | TS | maker- | AT3G1600 | \| Symbols: |
| | | | AAAGAAAGTTGTCA | AAT | | | N1- | 0.1 | MFP1 \| MAR |
| | | | GGAGGAGAAAGAG | | | | fgenesh- | | binding |
| | | | CAGTACTA[G/A]TTC | | | | gene- | | filament- |
| | | | TTGAGGAGACAAAG | | | | 23.12 | | like protein 1 \| |
| | | | GTTATAATCACCAGT | | | | | | chr3: |
| | | | TCTTGCTCCAGTTAT | | | | | | 5430889- |
| | | | TAT (SEQ ID NO: | | | | | | 5433817 REVERSE |
| | | | 48) | | | | | | LENGTH = 2929 |
| 23,257,618 | A | C | AGATAATGTGACTG | GAT -> | D -> A | TV | maker- | AT3G1592 | \| Symbols: |
| | | | ATTGGCATGAACTA | GCT | | | N1- | 0.1 | \| Phox (PX) |
| | | | ATCACAGAATCTGG | | | | augustus | | domain- |
| | | | ACTTCTTG[A/C]TAA | | | | -gene- | | containing |
| | | | GAGTCATTTTACTGA | | | | 23.138 | | protein \| |
| | | | TAGAGCTGCAGAAA | | | | | | chr3: |
| | | | CCGGAGAGGCATCA | | | | | | 5383596- |
| | | | ATAT (SEQ ID NO: | | | | | | 5387009 REVERSE |
| | | | 49) | | | | | | LENGTH = 3414 |
| 23,302,268 | G | A | CTGGAGCATGTACTC | CTA -> | None | TS | maker- | AT3G1583 | \| Symbols: |
| | | | CTCTACGCCCGTGAA | TTA | | | N1- | 0.1 | \| phosphatidic |
| | | | GAACAGAACTCCTA | | | | augustus | | acid |
| | | | CCGCTA[G/A]CAAAC | | | | -gene- | | phosphatase- |
| | | | ACGGTATCCAATGG | | | | 23.199 | | related/PAP2- |
| | | | ACTCTCACCACGTGG | | | | | | related \| |
| | | | ACAGGATTGCACAT | | | | | | chr3: |
| | | | CC (SEQ ID NO: | | | | | | 5354974- |
| | | | 50) | | | | | | 5356424 |
| | | | | | | | | | RD |
| | | | | | | | | | LENGTH = 1451 |
| 23,367,822 | T | C | GACTCAGTGAGAGC | TTT -> | None | TS | maker- | AT1G2803 | \| Symbols: |
| | | | CCAAGTCCGAAAAG | TTC | | | N1- | 0.1 | \| 2-oxoglutarate |
| | | | CCCTAGAAGAGTAC | | | | augustus | | (20G) and |
| | | | GGATGTTT[T/C]GAG | | | | -gene- | | Fe(II)-dependent |
| | | | GCCACGTTCGATGG | | | | 23.144 | | oxygenase |
| | | | AGTTTCAGCGGAGC | | | | | | superfamily |
| | | | TAAGGAAGGCTATT | | | | | | protein \| |
| | | | TTCAA (SEQ ID NO: | | | | | | chr1: |
| | | | 51) | | | | | | 9771793- |
| | | | | | | | | | 9773345 |
| | | | | | | | | | FORWARD |
| | | | | | | | | | LENGTH = 1553 |
| 23,380,089 | C | A | TCTAGGTTGCAGCCC | GTG -> | V -> L | TV | augustus | AT3G1554 | \| Symbols: |
| | | | AAATCCGGTAGCCTC | TTG | | | masked | 0.1 | IAA19, MSG2 \| |
| | | | GGATCTTTTCATAAT | | | | -N1- | | indole-3- |
| | | | CCTCA[C/A]CCTCCT | | | | abinit- | | acetic acid |
| | | | GCATGACTCTATGAA | | | | gene- | | inducible 19 \| |
| | | | CATTCTGCAAAATCA | | | | 23.488 | | chr3: |
| | | | AAATACCCAACCCA | | | | | | 5264024- |
| | | | (SEQ ID NO: | | | | | | 5265678 |
| | | | 177) | | | | | | RD |
| | | | | | | | | | LENGTH = 1655 |
| 23,457,696 | A | C | TGATCAGGCTTCTCT | AAT -> | N -> K | TV | augustus | AT3G1540 | \| Symbols: |
| | | | ATGTCATCAATCTTC | AAG | | | _masked | 0.2 | ATA20 \| |
| | | | TCTTTACCACCATCA | | | | -N1- | | anther 20 \| |
| | | | TTAAG[A/C]TTCTCT | | | | abinit- | | chr3: |
| | | | TTGTAATCAGGCATT | | | | gene- | | 5201644- |
| | | | ACTTTATGAGCATGC | | | | 23.498 | | 5203197 |
| | | | TTTTTTTCAGGATC | | | | | | RD |
| | | | (SEQ ID NO: | | | | | | LENGTH = 1554 |
| | | | 52) | | | | | | |
| 23,520,607 | G | A | AAAGAAGAGCTTAG | CTC -> | L -> F | TS | augustus | #N/A | #N/A |
| | | | TCAACGCATTGAAG | TTC | | | _masked | | |
| | | | AAAGATAGTCCGGT | | | | -N1- | | |
| | | | GGGCCAGA[G/A]TC | | | | abinit- | | |
| | | | TTGTTCTTTGTTTGA | | | | gene- | | |
| | | | ATTTGGCATTACCGG | | | | 23.516 | | |
| | | | GAAAGGCATGTGAG | | | | | | |
| | | | TGTG (SEQ ID NO: | | | | | | |
| | | | 53) | | | | | | |
| 23,552,773 | C | T | ACAGAACTGGTATT | CCG -> | P -> S | TS | maker- | AT3G1511 | \| Symbols: |
| | | | AATAGTAGTTGTAAT | TCG | | | N1- | 5.1 | \| unknown |
| | | | AACAATAGTGTCCA | | | | augustus | | protein; BEST |
| | | | GTGTTGT[C/T]CGAT | | | | -gene- | | Arabidopsis |
| | | | GGGAGGGAGTTTAC | | | | 23.153 | | thaliana |
| | | | AGAGAACTCAGACG | | | | | | protein |
| | | | TTACCTAGTTACATA | | | | | | match is: |
| | | | GGA (SEQ ID NO: | | | | | | unknown protein |
| | | | 54) | | | | | | (TAIR: |
| | | | | | | | | | AT1G53180.1); |
| | | | | | | | | | Has 47 |
| | | | | | | | | | Blast hits |
| | | | | | | | | | to 47 |
| | | | | | | | | | proteins in 15 |
| | | | | | | | | | species: |
| | | | | | | | | | Archae - 0; |
| | | | | | | | | | Bacteria - 0; |
| | | | | | | | | | Metazoa - 13; |
| | | | | | | | | | Fungi - 0; |
| | | | | | | | | | Plants - 30; |
| | | | | | | | | | Viruses - 0; |
| | | | | | | | | | Other |
| | | | | | | | | | Eukaryotes - 4 |
| | | | | | | | | | (source: |
| | | | | | | | | | NCBI BLink). \| |
| | | | | | | | | | chr3: |
| | | | | | | | | | 5085992- |
| | | | | | | | | | 5087489 REVERSE |
| | | | | | | | | | LENGTH = 1498 |
| 23,598,941 | A | G | CGGGGAGGACGGG | CTC -> | L -> A | subst | augustus | AT3G1492 | \| Symbols: |
| | | | AGGCTTTTTGACCTC | GCC | | | _masked | 0.1 | \| Peptide-N4- |
| | | | GAGGTAGGGAGTAG | | | | -N1- | | (N-acetyl-beta- |
| | | | GTGAGGTG[A/G]GG | | | | abinit- | | glucosaminyl) |
| | | | TTTTGGGATGGAGG | | | | gene- | | asparagine |
| | | | AGAGATGAGGAGA | | | | 23.533 | | amidase A |
| | | | GGGGAGAAATGTGG | | | | | | protein \| |
| | | | TGGTTTG (SEQ ID | | | | | | chr3: |
| | | | NO: | | | | | | 5018275- |
| | | | 55) | | | | | | 5020273 |
| | | | | | | | | | FORWARD |
| | | | | | | | | | LENGTH = 1999 |
| 23,670,623 | T | C | TGTGGCTTTGGGAA | CCA -> | None | TS | maker- | AT3G1464 | \| Symbols: |
| | | | ATCATAAACGTTGTT | CCG | | | N1- | 0.1 | CYP72A10 \| |
| | | | GAATACTTCTTTGAT | | | | augustus | | cytochrome |
| | | | TTGCTC[T/C]GGATC | | | | -gene- | | P450, |
| | | | CATTATGGTGATGGT | | | | 23.230 | | family 72, |
| | | | TGGTATAGGTCCAA | | | | | | subfamily A, |
| | | | ACCATGTATAGTAA | | | | | | polypeptide 10 \| |
| | | | GT (SEQ ID NO: | | | | | | chr3: |
| | | | 56) | | | | | | 4919856- |
| | | | | | | | | | 4921787 |
| | | | | | | | | | FORWARD |
| | | | | | | | | | LENGTH = 1932 |
| 23,682,848 | C | A | TGACGAGAGCACAG | CGA -> | R -> L | TV | maker- | AT3G5955 | \| Symbols: |
| | | | TCACGGAACAGATA | CTA | | | N1- | 0.1 | SYN3, ATRAD21.2, |
| | | | ATCAACTTGCTTTGA | | | | augustus | | ATSYN3 \| |
| | | | ATAGATT[C/A]GGAC | | | | -gene- | | Rad21/Rec8- |
| | | | AACACCAAGTAAAA | | | | 23.231 | | like family |
| | | | GATGACCAGACAAT | | | | | | protein \| |
| | | | CTCAATGCCAAGGT | | | | | | chr3: |
| | | | GCCT (SEQ ID NO: | | | | | | 21997054- |
| | | | 57) | | | | | | 22000678 |
| | | | | | | | | | RD |
| | | | | | | | | | LENGTH = 3625 |
| 23,745,365 | T | A | GGGATGCTAGGAAT | CAT -> | H -> Q | TV | maker- | AT3G1449 | \| Symbols: |
| | | | GTTTTTCGAGCCACG | CAA | | | N1- | 0.1 | \| Terpenoid |
| | | | ATATTCACTTGGAAG | | | | fgenesh- | | cyclases/Protein |
| | | | AATTCA[T/A]ACCGT | | | | gene- | | prenyltransferases |
| | | | TAAACTTACAATGGT | | | | 23.43 | | superfamily |
| | | | CCTCACTGTTGTGGA | | | | | | protein \| |
| | | | TGATACATGTGATGC | | | | | | chr3: |
| | | | (SEQ ID NO: | | | | | | 4863631- |
| | | | 58) | | | | | | 4865949 REVERSE |
| | | | | | | | | | LENGTH = 2319 |
| 23,792,572 | C | A | TCAATGGCGGCGGA | ACC -> | None | TV | fgenesh_ | AT3G1441 | \| Symbols: |
| | | | TCGGAGCAAAGGCG | ACA | | | masked- | 0.1 | \| Nucleotide/sugar |
| | | | TTGTGAGAGACGAG | | | | N1- | | transporter |
| | | | TTCGTGAC[C/A]TAT | | | | abinit- | | family protein \| |
| | | | GCTTACATTCTTCTCT | | | | gene- | | chr3: |
| | | | ACATCGCTCTCTCTA | | | | 23.282 | | 4815811- |
| | | | GCGGTCAAATCTTCT | | | | | | 4817980 |
| | | | T (SEQ ID NO: | | | | | | REVERSE |
| | | | 59) | | | | | | LENGTH = 2170 |
| 23,855,829 | G | A | CGGAACGGTCGACT | TGC -> | C -> Y | TS | fgenesh_ | AT3G1431 | \| Symbols: |
| | | | TCATCTTCGGAAACG | TAC | | | masked- | 0.1 | ATPME3, PME3 \| |
| | | | CCGCTGTCGTTCTCC | | | | N1- | | pectin |
| | | | AAAACT[G/A]CGAC | | | | abinit- | | methylesterase 3 \| |
| | | | ATCCACGCTCGCCGA | | | | gene- | | chr3: |
| | | | CCAAACTCCGGCCA | | | | 23.288 | | 4771902- |
| | | | GAAAAACATTGTCA | | | | | | 4775119 REVERSE |
| | | | CGG (SEQ ID NO: | | | | | | LENGTH = 3218 |
| | | | 60) | | | | | | |
| 23,910,029 | T | C | ATAGAGTCCGGTGG | AAT -> | None | TS | maker- | AT3G1423 | \| Symbols: |
| | | | TCAAGCTGAGAAGT | AAC | | | N1- | 0.3 | RAP2.2 \| related |
| | | | CTGCTAAGAGAAAG | | | | augustus | | to AP2 2 \| |
| | | | AGAAAGAA[T/C]CA | | | | -gene- | | chr3: |
| | | | GTACAGGGGGATTA | | | | 23.175 | | 4737146- |
| | | | GGCAGCGACCTTGG | | | | | | 4739136 REVERSE |
| | | | GGAAAATGGGCTGC | | | | | | LENGTH = 1991 |
| | | | TGAGAT (SEQ ID | | | | | | |
| | | | NO: | | | | | | |
| | | | 61) | | | | | | |
| 23,947,522 | C | T | ACGACGACCAGATC | CCG -> | P -> S | TS | maker- | AT3G1413 | \| Symbols: |
| | | | CGCTCTCACTCCGAC | TCG | | | N1- | 0.1 | \| Aldolase- |
| | | | TCACCCGATCCGTCT | | | | augustus | | type TIM barrel |
| | | | TCTTCT[C/T]CGCCG | | | | -gene- | | family protein \| |
| | | | CCGCCGTCGGGAAA | | | | 23.179 | | chr3: |
| | | | AGTCACGGTAACGG | | | | | | 4685646- |
| | | | TGGCTTTCCCAGGTC | | | | | | 4688309 |
| | | | CG (SEQ ID NO: | | | | | | REVERSE |
| | | | 62) | | | | | | LENGTH = 2664 |
| 24,021,883 | A | C | GAGAATCTTGAAAG | GTT -> | V -> G | TV | maker- | AT3G1396 | \| Symbols: |
| | | | CTTCTAGAAGCTTCT | GGT | | | N1- | 0.1 | AtGRF5, GRF5 \| |
| | | | GGAAAAAAAGTTCT | | | | augustus | | growth- |
| | | | TTCCCCA[A/C]CTCC | | | | -gene- | | regulating |
| | | | AACTCTCTCCCCAAT | | | | 24.75 | | factor 5 \| |
| | | | CCCTTGAAAATATCT | | | | | | chr3: |
| | | | ACAATTAATCATAAT | | | | | | 4608383- |
| | | | T (SEQ ID NO: | | | | | | 4610399 |
| | | | 63) | | | | | | RD |
| | | | | | | | | | LENGTH = 2017 |
| 24,056,999 | G | A | CACAGAACGCTAAC | TCC -> | S -> F | TS | fgenesh | AT3G1388 | \| Symbols: \| |
| | | | GCGCCAGCGTAAGT | TTC | | | masked- | 0.1 | Tetratricopeptide |
| | | | GAACTTATCGAGCTT | | | | N1- | | repeat |
| | | | CAAATTG[G/A]AATC | | | | abinit- | | (TPR)-like |
| | | | TCTAGCTTCTAGGAA | | | | gene- | | superfamily |
| | | | CAGCTTCATGGAGT | | | | 24.382 | | protein \| |
| | | | GTTCGTGAAACCCA | | | | | | chr3: |
| | | | ACT (SEQ ID NO: | | | | | | 4572180- |
| | | | 64) | | | | | | 4574490 |
| | | | | | | | | | RD |
| | | | | | | | | | LENGTH = 2311 |
| 24,111,397 | C | T | GGCAGAGACTAAAG | GCA -> | A -> T | TS | maker- | AT2G2847 | \| Symbols: |
| | | | AGACAAGAAACCCT | ACA | | | N1- | 0.2 | BGAL8 \| beta- |
| | | | AGAAGAAACAGAGC | | | | augustus | | galactosidase 8\| |
| | | | AGAAGCTG[C/T]TGC | | | | -gene- | | chr2: |
| | | | CATAGCCACATTCTT | | | | 24.79 | | 12168915- |
| | | | CATTACTGTTGAAAG | | | | | | 12173679 |
| | | | CAAAATATCTTGCTT | | | | | | REVERSE |
| | | | TA (SEQ ID NO: | | | | | | LENGTH = 4765 |
| | | | 65) | | | | | | |
| 24,153,185 | A | T | TAGTAGTGAATGCG | TAT -> | Y -> F | TV | augustus | AT3G1368 | \| Symbols: |
| | | | AGCATTTGATCTCCG | TTT | | | masked | 2.1 | LDL2 \| LSD1- |
| | | | CTGCTTATGAGTTTC | | | | -N1- | | like2 \| |
| | | | TGTTGT[A/T]TAAT | | | | abinit- | | chr3: |
| | | | GGTTTCATTAACTTT | | | | gene- | | 4479193- |
| | | | GGGGTGTCTCCGTTG | | | | 24.481 | | 4481509 |
| | | | TTTAATGGATATGTT | | | | | | REVERSE |
| | | | C | | | | | | LENGTH = 2317 |
| | | | (SEQ ID NO: | | | | | | |
| | | | 66) | | | | | | |
| 24,207,903 | C | T | CGATGAAATTTGATC | CCA -> | P -> S | TS | augustus | AT3G1368 | \| Symbols: |
| | | | TTCAAGGAATTGGA | TCA | | | masked | 0.1 | \| F-box and |
| | | | AACGAAAGAGACTT | | | | -N1- | | associated |
| | | | CATTGAT[C/T]CATC | | | | abinit- | | interaction |
| | | | TATAAAGCAAGTAA | | | | gene- | | domains- |
| | | | GTATACTTGACCAAG | | | | 24.492 | | containing |
| | | | TAGAGATCACTCAA | | | | | | protein\| |
| | | | GTA (SEQ ID NO: | | | | | | chr3: |
| | | | 67) | | | | | | 4477289- |
| | | | | | | | | | 4478721 REVERSE |
| | | | | | | | | | LENGTH = 1433 |
| 24,242,389 | G | T | GGAGGAGGGAAGA | AAG -> | K -> N | TV | augustus | AT3G1336 | \| Symbols: |
| | | | TTGATTCAGGTAGCC | AAT | | | masked | 0.1 | WIP3 \| WPP domain |
| | | | AAGGGAGAGATACC | | | | -N1- | | interacting |
| | | | ATCAAGAA[G/T]GG | | | | abinit- | | protein 3 \| |
| | | | GAGTGAAGAGCGCA | | | | gene- | | chr3: |
| | | | TAGATTCGGACTTGA | | | | 24.500 | | 4338359- |
| | | | GAAGTAGCGATTTT | | | | | | 4340412 REVERSE |
| | | | GTGTT (SEQ ID NO: | | | | | | LENGTH = 2054 |
| | | | 68) | | | | | | |
| 24,304,491 | G | C | GCTACGTTGCGCCGT | GAA -> | E -> Q | TV | maker- | AT3G1308 | \| Symbols: |
| | | | ATCTCATGGACAGTT | CAA | | | N1- | 0.4 | ATMRP3, |
| | | | TTGTTCAGTACCTGA | | | | augustus | | MRP3, ABCC3 \| |
| | | | ACGGT[G/C]AAAGG | | | | -gene- | | multidrug |
| | | | CAATACAAATACCAA | | | | 24.24 | | resistance- |
| | | | GGATACGTTTTGGTA | | | | | | associated protein |
| | | | ACGATTTTCTTCGTT | | | | | | 3 \| |
| | | | (SEQ ID NO: | | | | | | chr3: |
| | | | 69) | | | | | | 4195799- |
| | | | | | | | | | 4201265 REVERSE |
| | | | | | | | | | LENGTH = 5467 |
| 24,359,101 | T | G | CTCAATGATGAAGA | ATC -> | I -> S | TV | augustus | AT3G1295 | \| Symbols: |
| | | | AGATGAGAATGATG | AGC | | | masked | 5.1 | \| SAUR-like |
| | | | ATGCTGTTAAGGAG | | | | -N1- | | auxin-responsive |
| | | | ATGCAAGA[T/G]CG | | | | abinit- | | protein family \| |
| | | | TGTCCACACAATTAA | | | | gene- | | chr3: |
| | | | GTCGTTCATATTCCT | | | | 24.530 | | 4135500- |
| | | | ACACAAGCCTCAGA | | | | | | 4136143 |
| | | | TCCC (SEQ ID NO: | | | | | | REVERSE |
| | | | 70) | | | | | | LENGTH = 644 |
| 24,362,374 | T | A | ATCATATGGGGAGG | GGT -> | None | TV | augustus | AT3G1295 | \| Symbols: |
| | | | CACTGGTAGCCGTG | GGA | | | masked | 0.1 | \| Trypsin |
| | | | GGAGGTTGAAGCTG | | | | -N1- | | family protein \| |
| | | | AAAGTAGG[T/A]GA | | | | abinit- | | chr3: |
| | | | GTCTCCAGAGAGCT | | | | gene- | | 4132528- |
| | | | GGACTACTGGAGTT | | | | 24.531 | | 4135134 REVERSE |
| | | | GATCTGGGAAGGTT | | | | | | LENGTH = 2607 |
| | | | ACTTAC (SEQ ID NO: | | | | | | |
| | | | 71) | | | | | | |
| 24,456,688 | A | C | AAAGCTGCTTCGATC | AAT -> | N -> K | TV | augustus | AT3G1276 | \| Symbols: |
| | | | TGCCAATCACTGGCT | AAG | | | masked | 0.1 | \| CONTAINS |
| | | | TTAAGAGCCTGAAG | | | | -N1- | | InterPro |
| | | | AGCACT[A/C]TTCTC | | | | abinit- | | DOMAIN/s: |
| | | | ACTGCTCCCCCAGCA | | | | gene- | | Defective- |
| | | | CATTATTAAACAAAT | | | | 24.551 | | in-cullin |
| | | | CACACACACAAAAA | | | | | | neddylation |
| | | | A (SEQ ID NO: | | | | | | protein |
| | | | 72) | | | | | | (InterPro: |
| | | | | | | | | | IPR014764), |
| | | | | | | | | | Protein of |
| | | | | | | | | | unknown |
| | | | | | | | | | function |
| | | | | | | | | | DUF298 |
| | | | | | | | | | (InterPro: |
| | | | | | | | | | IPR005176), |
| | | | | | | | | | UBA-like |
| | | | | | | | | | (InterPro: |
| | | | | | | | | | IPR009060); |
| | | | | | | | | | BEST |
| | | | | | | | | | Arabidopsis |
| | | | | | | | | | thaliana |
| | | | | | | | | | protein |
| | | | | | | | | | match is: |
| | | | | | | | | | Domain of |
| | | | | | | | | | unknown function |
| | | | | | | | | | (DUF298) |
| | | | | | | | | | (TAIR: |
| | | | | | | | | | AT1G15860.2); |
| | | | | | | | | | Has 857 Blast |
| | | | | | | | | | hits |
| | | | | | | | | | to 855 proteins |
| | | | | | | | | | in 202 species: |
| | | | | | | | | | Archae -0; |
| | | | | | | | | | Bacteria - 0; |
| | | | | | | | | | Metazoa - 482; |
| | | | | | | | | | Fungi - 154; |
| | | | | | | | | | Plants - 139; |
| | | | | | | | | | Viruses - 0; |
| | | | | | | | | | Other |
| | | | | | | | | | Eukaryotes - 82 |
| | | | | | | | | | (source: |
| | | | | | | | | | NCBI BLink). \| |
| | | | | | | | | | chr3: |
| | | | | | | | | | 4054739- |
| | | | | | | | | | 4056980 |
| | | | | | | | | | RD |
| | | | | | | | | | LENGTH = 2242 |
| 24,500,169 | C | T | GTAAATAAAGAGTA | AGG -> | R -> K | TS | maker- | AT3G1264 | \| Symbols: |
| | | | ACAATCATTACATTG | AAG | | | N1- | 0.1 | \| RNA binding |
| | | | GCGACAAAGATTGT | | | | augustus | | (RRM/RBD/RNP |
| | | | TCGAGAC[C/T]TAGC | | | | -gene- | | motifs) |
| | | | ATCTTCCAAAGGGC | | | | 24.103 | | family |
| | | | GGCTAGTAGATAAT | | | | | | protein \| |
| | | | GTCCCTACAAATACC | | | | | | chr3: |
| | | | AGT (SEQ ID NO: | | | | | | 4014213- |
| | | | 73) | | | | | | 4017869 |
| | | | | | | | | | RD |
| | | | | | | | | | LENGTH = 3657 |

TABLE 12

| | Ref | Alt | | Codon | AA | | Affected | | |
|---|---|---|---|---|---|---|---|---|---|
| Position | nt | nt | Sequence | Change | Change | Type | gene | Ortholog | Description |
| 23,089,542 | C | G | CTTGAAAACGTAGA | GAC | D | TV | augustus_ | AT3G163 | \| Symbols: |
| | | | AGAAAGCGTTGCG | -> | -> H | | masked- | 40.2 | PDR1 \| |
| | | | TTTTATGAGAAGCA | CAC | | | N1- | | pleiotropic |
| | | | GTTCTCTGT[C/G]C | | | | abinit- | | drug |
| | | | CAACAGATCTTGAA | | | | gene- | | resistance 1 \| |
| | | | GAGGTCAGATTTG | | | | 23.422 | | chr3: |
| | | | GGGACAGAGTGTT | | | | | | 5539788- |
| | | | TGTTGAATA (SEQ | | | | | | 5546449 |
| | | | ID NO: | | | | | | FORWARD |
| | | | 74) | | | | | | LENGTH = 6662 |
| 23,089,635 | A | T | GTTGAATACGAGA | TCC | S | TV | augustus_ | AT3G163 | \| Symbols: |
| | | | GAAGCTGGATGTG | -> | -> T | | masked- | 40.2 | PDR1 \| |
| | | | ATTTGAATCTGTCG | ACC | | | N1- | | pleiotropic |
| | | | TAAGGGACGG[A/T | | | | abinit- | | drug |
| | | | GATTGGCACCGACA | | | | gene- | | resistance 1 \| |
| | | | TGGAAGGTTTTGG | | | | 23.422 | | chr3: |
| | | | ACTGTTTGG (SEQ | | | | | | 5539788- |
| | | | ID NO: | | | | | | 5546449 |
| | | | 75) | | | | | | FORWARD |
| | | | | | | | | | LENGTH = 6662 |
| 23,090,743 | T | G | AATGGGGAAAACT | ATT | I | TV | augustus_ | AT3G163 | \| Symbols: |
| | | | AGACCTGTTGTGAC | -> | -> L | | masked | 40.2 | PDR1 \| |
| | | | TCTTTTCTTTTGACC | CTT | | | - | | pleiotropic |
| | | | ACCTGAAA[T/G]TC | | | | N1- | | drug |
| | | | CTCGTGTCATTTCG | | | | abinit- | | resistance 1 \| |
| | | | TCTCCGACCATCGT | | | | gene- | | chr3: |
| | | | ATCTTTGCATAAGT | | | | 23.422 | | 5539788- |
| | | | CAAGCC (SEQ ID | | | | | | 5546449 |
| | | | NO: | | | | | | FORWARD |
| | | | 76) | | | | | | LENGTH = 6662 |
| 23,090,785 | A | T | ACCTGAAATTCCTC | TTA | L | TV | augustus_ | AT3G163 | \| Symbols: |
| | | | GTGTCATTTCGTCT | -> | -> 1 | | masked- | 40.2 | PDR1 \| |
| | | | CCGACCATCGTATC | ATA | | | N1- | | pleiotropic |
| | | | TTTGCATA[A/T]GT | | | | abinit- | | drug |
| | | | CAAGCCCTAATATC | | | | gene- | | resistance 1 \| |
| | | | TAAAATAGAAAATG | | | | 23.422 | | chr3: |
| | | | ATCAATAGTTGCCC | | | | | | 5539788- |
| | | | AAATTT (SEQ ID | | | | | | 5546449 |
| | | | NO: | | | | | | FORWARD |
| | | | 77) | | | | | | LENGTH = 6662 |
| 23,091,367 | T | C | GTTAGAGATATTAA | TAC | Y | TS | augustus_ | AT3G163 | \| Symbols: |
| | | | ATCTAGGTTAAAAG | -> | -> C | | masked- | 40.2 | PDR1 \| |
| | | | TGAGATCATTACTT | TGC | | | N1- | | pleiotropic |
| | | | TGAGAGTG[T/C]AG | | | | abinit- | | drug |
| | | | TCTGTGATGAGACT | | | | gene- | | resistance 1 \| |
| | | | GCTCTTGACATTCC | | | | 23.422 | | chr3: |
| | | | CAGCTGCGATGGA | | | | | | 5539788- |
| | | | CTTCATG (SEQ ID | | | | | | 5546449 |
| | | | NO: | | | | | | FORWARD |
| | | | 78) | | | | | | LENGTH = 6662 |
| 23,092,042 | A | G | GAGTTAATTACCTT | ATT | I | TS | augustus_ | AT3G163 | \| Symbols: |
| | | | GAGGGTTTCATGAT | -> | -> T | | masked- | 40.2 | PDR1 \| |
| | | | ACCAGAAGCTTCTC | ACT | | | N1- | | pleiotropic |
| | | | TAAGAATA[A/G]TG | | | | abinit- | | drug |
| | | | AGTTTAGTGGTTTT | | | | gene- | | resistance 1 \| |
| | | | TGCAAAGTTTAAAC | | | | 23.422 | | chr3: |
| | | | CAAATAAACGAAG | | | | | | 5539788- |
| | | | GCCTCTC (SEQ ID | | | | | | 5546449 |
| | | | NO: | | | | | | FORWARD |
| | | | 79) | | | | | | LENGTH = 6662 |
| 23,150,402 | C | G | GAGTGCAAGTGGA | GGA | G | TV | maker- | AT3G161 | \| Symbols: |
| | | | ACTGCTACGCCACC | -> | -> R | | N1- | 70.1 | \|AMP- |
| | | | AGTAAACCAAGTCC | CGA | | | augustus- | | dependent |
| | | | CAAGGACTC[C/G]T | | | | gene- | | synthetase |
| | | | GCAACAAACTCAGC | | | | 23.190 | | and ligase |
| | | | TGATGGTTTCGCCA | | | | | | family |
| | | | CAATTCCAACTCTA | | | | | | protein \| |
| | | | GCTCCTT (SEQ ID | | | | | | chr3: |
| | | | NO: | | | | | | 5476074- |
| | | | 80) | | | | | | 5480302 FORWARD |
| | | | | | | | | | LENGTH = 4229 |
| 23,150,595 | TC | CA | GGATAGGTTAGAA | GAT | D | Subst | maker- | AT3G161 | \| Symbols: |
| | | | GTAGAAATAGCAA | -> | -> C | | N1- | 70.1 | \| AMP- |
| | | | CAAAAATTCAAACA | TGT | | | augustus- | | dependent |
| | | | GATCACCATA[TC/C | | | | gene- | | synthetase |
| | | | A]TACATTITTAGTA | | | | 23.190 | | and ligase |
| | | | TCTTCCTTGTGGAA | | | | | | family |
| | | | CAACTTAGAGATGG | | | | | | protein \| chr3: |
| | | | TAAAGGCAG (SEQ | | | | | | 5476074- |
| | | | ID NO: | | | | | | 5480302 FORWARD |
| | | | 81) | | | | | | LENGTH = 4229 |
| 23,155,220 | G | T | CCACAAACCACCTC | GAC | D | TV | maker- | AT3G161 | \| Symbols: |
| | | | TCCTTTATTCGACA | -> | -> E | | N1- | 50.1 | \| N- |
| | | | CGGCAATGAGTCCA | GAA | | | augustus- | | terminal |
| | | | GCGAACCC[G/T]TC | | | | gene- | | nucleophile |
| | | | GTCGAGCCGATGCT | | | | 23.191 | | aminohydrolases |
| | | | TGATGACGTAATCA | | | | | | (Ntn hydrolases) |
| | | | ACCGCTTCTTGTAG | | | | | | superfamily |
| | | | CCCAAC (SEQ ID | | | | | | protein \| chr3: |
| | | | NO: | | | | | | 5471735- |
| | | | 82) | | | | | | 5473276 FORWARD |
| | | | | | | | | | LENGTH = 1542 |
| 23,155,766 | C | A | GCTTCTTTGGCCAG | GAG | E | TV | maker- | AT3G161 | \| Symbols: |
| | | | CTTGAGCATCCCCA | -> | -> D | | N1- | 50.1 | \| N- |
| | | | CGTTGTCTTCCGTG | GAT | | | augustus- | | terminal |
| | | | ACGAAGTA[C/A]TC | | | | gene- | | nucleophile |
| | | | GTTGTCCACAGTTT | | | | 23.191 | | aminohydrolases |
| | | | CAACTCCCTAATAA | | | | | | (Ntn hydrolases) |
| | | | AAACCAAATCGATT | | | | | | superfamily |
| | | | TCGGTT (SEQ ID | | | | | | protein \| chr3: |
| | | | NO: | | | | | | 5471735- |
| | | | 83) | | | | | | 5473276 FORWARD |
| | | | | | | | | | LENGTH = 1542 |
| 23,314,197 | T | C | CAGCAAGTGACCA | AAC | N | TS | augustus_ | AT3G157 | \| Symbols: |
| | | | GGAAGGTCATGTTC | -> | -> D | | masked- | 30.1 | PLDALPHA1, |
| | | | CAGTGACTCACTTG | GAC | | | N1- | | PLD \| |
| | | | AGTAAAAGT[T/C]C | | | | abinit- | | phospholipase |
| | | | CAGTACTTATCAGC | | | | gene- | | D alpha 1 \| chr3: |
| | | | AATACGGTTAACTT | | | | 23.470 | | 5330322- |
| | | | TCTCAATGCATTCC | | | | | | 5333745 FORWARD |
| | | | AAGCTTG (SEQ ID | | | | | | LENGTH = 3424 |
| | | | NO: | | | | | | |
| | | | 84) | | | | | | |
| 23,318,357 | C | T | TTGTAGATCGATCG | GTT | V | TS | augustus_ | AT3G157 | \| Symbols: |
| | | | TTGCGTAGAGCTGT | -> | -> 1 | | masked- | 30.1 | PLDALPHA1, |
| | | | GTTTCTCCTTTGCC | ATT | | | N1- | | PLD \| |
| | | | GAAACCAA[C/T]TG | | | | abinit- | | phospholipase |
| | | | CCTCTTCAACATTT | | | | gene- | | D alpha 1 \| |
| | | | GCTCTAATCTGGAA | | | | 23.470 | | chr3: |
| | | | TTTAAAACAGAGGT | | | | | | 5330322- |
| | | | TAAAGA (SEQ ID | | | | | | 5333745 |
| | | | NO: | | | | | | FORWARD |
| | | | 85) | | | | | | LENGTH = 3424 |
| 23,343,089 | C | T | TGGTGCTTTCCTTT | AGG | None | TS | fgenesh | AT3G156 | \| Symbols: |
| | | | AGGCCTCACAACAT | -> | | | maske | 50.1 | \| alpha/beta- |
| | | | AAGTCCTTCCAAAC | AGA | | | d- | | Hydrolases |
| | | | TCATATCC[C/T]CTT | | | | N1- | | superfamily |
| | | | GTACTTCTACTACC | | | | abinit- | | protein \| chr3: |
| | | | TACAAATATTTTGC | | | | gene- | | 5305926- |
| | | | CAGAAACATATTCA | | | | 23.322 | | 5307968 FORWARD |
| | | | AGCAG (SEQ ID | | | | | | LENGTH = 2043 |
| | | | NO: | | | | | | |
| | | | 86) | | | | | | |
| 23,679,276 | T | G | GGAAGTCGAGAGA | TTT | F | TV | maker- | AT1G535 | \| Symbols: |
| | | | TGATGAGTGCTCAA | -> | -> V | | N1- | 90.1 | NTMC2TYPE6.1, |
| | | | CGGTTCCTGAGAAT | GTT | | | augustus- | | NTMC2T6.1 \| |
| | | | GAGTCTGTG[T/G]T | | | | gene- | | Calcium- |
| | | | TGGTTCAGAATGTC | | | | 23.161 | | dependent lipid- |
| | | | AGTTTGAATATTCT | | | | | | binding (CaLB |
| | | | GATGATGATGATAC | | | | | | domain) |
| | | | TGCACAT (SEQ ID | | | | | | family |
| | | | NO: | | | | | | protein \| |
| | | | 87) | | | | | | chr1: |
| | | | | | | | | | 19996315- |
| | | | | | | | | | 20000265 FORWARD |
| | | | | | | | | | LENGTH = 3951 |
| 23,679,287 | A | T | GATGATGAGTGCTC | GAA | E | TV | maker- | AT1G535 | \| Symbols: |
| | | | AACGGTTCCTGAGA | -> | -> D | | N1- | 90.1 | NTMC2TYPE6.1, |
| | | | ATGAGTCTGTGTTT | GAT | | | augustus- | | NTMC2T6.1 \| |
| | | | GGTTCAGA[A/T]TG | | | | gene- | | Calcium- |
| | | | TCAGTTTGAATATT | | | | 23.161 | | dependent |
| | | | CTGATGATGATGAT | | | | | | lipid- |
| | | | ACTGCACATGGAA | | | | | | binding (CaLB |
| | | | GTGTGCA (SEQ ID | | | | | | domain) |
| | | | NO: | | | | | | family |
| | | | 88) | | | | | | protein \| chr1: |
| | | | | | | | | | 19996315- |
| | | | | | | | | | 20000265 FORWARD |
| | | | | | | | | | LENGTH = 3951 |
| 23,679,396 | C | A | CAAGGAAAGCAAA | CTT | L | TV | maker- | AT1G535 | \| Symbols: |
| | | | AGATGAAGGTAAA | -> | -> 1 | | N1- | 90.1 | NTMC2TYPE6.1, |
| | | | GGTGTGAGGGCAG | ATT | | | augustus- | | NTMC2T6.1 \| |
| | | | GAGAAGATGGT[C/ | | | | gene- | | Calcium- |
| | | | A]TTGTGAATACAT | | | | 23.161 | | dependent lipid- |
| | | | CAGCAAACTCTAAA | | | | | | binding (CaLB |
| | | | GAAGATTCTAGAG | | | | | | domain) family |
| | | | GTGTTACACAT | | | | | | protein \| chr1: |
| | | | (SEQ ID NO: | | | | | | 19996315- |
| | | | 89) | | | | | | 20000265 FORWARD |
| | | | | | | | | | LENGTH = 3951 |
| 23,886,929 | G | A | CGATCCTCGCTACG | TAC | None | TS | maker- | AT3G142 | \| Symbols: |
| | | | GAACTCTCCACTTC | -> | | | N1- | 70.1 | FAB1B \|phosphati- |
| | | | CAAAGCTAGTGGA | TAT | | | augustus- | | dylinositol- |
| | | | AGGCCGCAA[G/A]T | | | | gene- | | 4-phosphate 5- |
| | | | ATCCCCACGCACCT | | | | 23.235 | | kinase family |
| | | | GAGGCATCTCCCTC | | | | | | protein \| chr3: |
| | | | ATTATCTTCTTCCTC | | | | | | 4753925- |
| | | | GTCAAA (SEQ ID | | | | | | 4761456 FORWARD |
| | | | NO: | | | | | | LENGTH = 7532 |
| | | | 90) | | | | | | |
| 23,925,895 | C | A | AGATAAAAACCGA | CCA | P | TV | augustus_ | AT3G142 | \| Symbols: |
| | | | ACCTTTTGGAAGGT | -> | -> Q | | masked- | 05.1 | \| Phosphoinositide |
| | | | TCTCAAGCTAGACA | CAA | | | N1- | | phosphatase |
| | | | GAACAGAGC[C/A] | | | | abinit- | | family |
| | | | AACAGAGCTCAACT | | | | gene- | | protein \| |
| | | | TTCACCAAGACTCA | | | | 23.587 | | chr3: |
| | | | ACTCCTTACACAGA | | | | | | 4715707- |
| | | | ATCCGAAT (SEQ ID | | | | | | 4720951 REVERSE |
| | | | NO: | | | | | | LENGTH = 5245 |
| | | | 91) | | | | | | |
| 23,963,309 | CTT | - | CACATTAAACCGGA | GCT,TC | AS | Deletion | augustus_ | AT3G140 | \| Symbols: |
| | | - | AGCTAATCGCCGGT | T | -> A | (tandem | masked- | 75.2 | \| Mono- |
| | | - | GTCGACGACGAGA | -> | | repeat) | N1- | | /di- |
| | | | GCTCCGAAG[CTT/] | GCT | | | abinit- | | acylglycerol |
| | | | CTTCTTCTTCTACTA | | | | gene- | | lipase, |
| | | | CTCATGGTGCTTCT | | | | 23.596 | | N- |
| | | | TTGAGGATTGATCG | | | | | | terminal; |
| | | | TGTTTCG (SEQ ID | | | | | | Lipase, |
| | | | NO: | | | | | | class 3 \| |
| | | | 92) | | | | | | chr3: |
| | | | | | | | | | 4663569- |
| | | | | | | | | | 4667079 REVERSE |
| | | | | | | | | | LENGTH = 3511 |
| 24,029,270 | G | A | ATTATCTATTGATA | CCT | P | TS | maker- | AT3G139 | \| Symbols: |
| | | | CCTGAGTCTTAAGC | -> | -> S | | N1- | 50.1 | \| unknown |
| | | | CGCTGATGACTAGT | TCT | | | augustus- | | protein; BEST |
| | | | AGAATGAG[G/A]CT | | | | gene- | | Arabidopsis |
| | | | CATCGAAGCAGAA | | | | 24.76 | | thaliana |
| | | | AATAAAACGGTACT | | | | | | protein |
| | | | GCTAATAACCAATC | | | | | | match is: |
| | | | CAAGATA (SEQ ID | | | | | | unknown |
| | | | NO: | | | | | | protein (TAIR: |
| | | | 93) | | | | | | AT4G13266.1); |
| | | | | | | | | | Has 339 Blast |
| | | | | | | | | | hits to 265 |
| | | | | | | | | | proteins in 12 |
| | | | | | | | | | species: |
| | | | | | | | | | Archae -0; |
| | | | | | | | | | Bacteria - 0; |
| | | | | | | | | | Metazoa - 0; |
| | | | | | | | | | Fungi - 0; |
| | | | | | | | | | Plants - 339; |
| | | | | | | | | | Viruses - 0; |
| | | | | | | | | | Other |
| | | | | | | | | | Eukaryotes - 0 |
| | | | | | | | | | (source: |
| | | | | | | | | | NCBI BLink). \| |
| | | | | | | | | | chr3: |
| | | | | | | | | | 4604149- |
| | | | | | | | | | 4605425 FORWARD |
| | | | | | | | | | LENGTH = 1277 |
| 24,029,279 | A | T | TGATACCTGAGTCT | TTC | F | TV | maker- | AT3G139 | \| Symbols: |
| | | | TAAGCCGCTGATGA | -> | -> 1 | | N1- | 50.1 | \| unknown |
| | | | CTAGTAGAATGAG | ATC | | | augustus- | | protein; BEST |
| | | | GCTCATCGA[A/T]G | | | | gene- | | Arabidopsis |
| | | | CAGAAAATAAAAC | | | | 24.76 | | thaliana |
| | | | GGTACTGCTAATAA | | | | | | protein |
| | | | CCAATCCAAGATAT | | | | | | match is: |
| | | | ATAGAGAC (SEQ ID | | | | | | unknown |
| | | | NO: | | | | | | protein (TAIR: |
| | | | 94) | | | | | | AT4G13266.1); |
| | | | | | | | | | Has 339 Blast |
| | | | | | | | | | hits to 265 |
| | | | | | | | | | proteins in 12 |
| | | | | | | | | | species: |
| | | | | | | | | | Archae - 0; |
| | | | | | | | | | Bacteria - 0; |
| | | | | | | | | | Metazoa - 0; |
| | | | | | | | | | Fungi - 0; |
| | | | | | | | | | Plants - 339; |
| | | | | | | | | | Viruses - 0; |
| | | | | | | | | | Other |
| | | | | | | | | | Eukaryotes - 0 |
| | | | | | | | | | (source: |
| | | | | | | | | | NCBI BLink). \| |
| | | | | | | | | | chr3: |
| | | | | | | | | | 4604149- |
| | | | | | | | | | 4605425 FORWARD |
| | | | | | | | | | LENGTH = 1277 |
| 24,029,294 | G | T | AAGCCGCTGATGAC | CGT | R | TV | maker- | AT3G139 | \| Symbols: |
| | | | TAGTAGAATGAGG | -> | -> S | | N1- | 50.1 | \| unknown |
| | | | CTCATCGAAGCAGA | AGT | | | augustus- | | protein; BEST |
| | | | AAATAAAAC[G/T]G | | | | gene- | | Arabidopsis |
| | | | TACTGCTAATAACC | | | | 24.76 | | thaliana |
| | | | AATCCAAGATATAT | | | | | | protein match is: |
| | | | AGAGACCACGCTTG | | | | | | unknown protein |
| | | | GAACTCT (SEQ ID | | | | | | (TAIR: |
| | | | NO: | | | | | | AT4G13266.1); |
| | | | 95) | | | | | | Has 339 Blast |
| | | | | | | | | | hits |
| | | | | | | | | | to 265 proteins |
| | | | | | | | | | in 12 |
| | | | | | | | | | species: |
| | | | | | | | | | Archae - 0; |
| | | | | | | | | | Bacteria - 0; |
| | | | | | | | | | Metazoa - 0; |
| | | | | | | | | | Fungi - 0; |
| | | | | | | | | | Plants - 339; |
| | | | | | | | | | Viruses - 0; |
| | | | | | | | | | Other |
| | | | | | | | | | Eukaryotes - 0 |
| | | | | | | | | | (source: |
| | | | | | | | | | NCBI BLink). \| |
| | | | | | | | | | chr3: |
| | | | | | | | | | 4604149- |
| | | | | | | | | | 4605425 |
| | | | | | | | | | FORWARD |
| | | | | | | | | | LENGTH = 1277 |
| 24,078,887 | C | T | CCTCTGTATTTGAC | TCA | S | TS | maker- | AT3G076 | \| Symbols: |
| | | | ATCTCTTACATCTAT | -> | -> L | | N1- | 90.1 | \| 6- |
| | | | AAATGTCCCTTATT | TTA | | | augustus- | | phosphogluconate |
| | | | TTTACTT[C/T]ACTG | | | | gene- | | dehydrogenase |
| | | | TAAGTTTGGACCCA | | | | 24.6 | | family protein \| |
| | | | AATCTTGTCTTGTT | | | | | | chr3: |
| | | | GAAAATTCTGCCAT | | | | | | 2457108- |
| | | | TGAA (SEQ ID NO: | | | | | | 2459552 |
| | | | 96) | | | | | | FORWARD |
| | | | | | | | | | LENGTH = 2445 |
| 24,355,223 | T | C | GGTTTCGGCCTTTC | None | None | TS | fgenesh | AT3G132 | \| Symbols: |
| | | | AAAAAAAACTGTTA | | | | _masked- | 20.1 | WBC27, |
| | | | ACCAAATTTCGAAA | | | | N1- | | ABCG26 \| ABC- |
| | | | TGAACGAA[T/C]CG | | | | abinit- | | 2 type |
| | | | AATTAATCAAAATT | | | | gene- | | transporter |
| | | | TTGAACTGAAATTA | | | | 24.288 | | family |
| | | | CCATAGTTTAATTG | | | | | | protein \| |
| | | | AATTTT (SEQ ID | | | | | | chr3: |
| | | | NO: | | | | | | 4247968- |
| | | | 97) | | | | | | 4250703 |
| | | | | | | | | | REVERSE |
| | | | | | | | | | LENGTH = 2736 |
| 24,497,060 | A | T | CGCAAACGCCAAGT | TCA | S | TV | maker- | AT3G126 | \| Symbols: |
| | | | CAGGAGCATTAATC | -> | -> T | | N1- | 60.1 | FLA14 \| |
| | | | GCTGAAACTCCACT | ACA | | | augustus- | | FASCICLIN- |
| | | | TGTTGTTG[A/T]GT | | | | gene- | | like |
| | | | TGGCTTTTGCAGCA | | | | 24.102 | | arabinogalactan |
| | | | GAAGGTGGCTTAG | | | | | | protein 14 |
| | | | CAGTAGCGGCAGT | | | | | | precursor \| |
| | | | AGCAGCAG (SEQ ID | | | | | | chr3: |
| | | | NO: | | | | | | 4019060- |
| | | | 98) | | | | | | 4019827 |
| | | | | | | | | | FORWARD |
| | | | | | | | | | LENGTH = 768 |
| 24,542,627 | T | A | GAGGTTTCTACACT | TGC | C | TV | maker- | AT3G125 | \| Symbols: |
| | | | TATGATGCCTTTAT | -> | -> S | | N1- | 00.1 | ATHCHIB, PR3, PR- |
| | | | CACCGCCGCTAAAT | AGC | | | augustus- | | 3, CHI- |
| | | | ATTTCCCT[T/A]GCT | | | | gene- | | B, B- |
| | | | TCTGCAACAATGGA | | | | 24.42 | | CHI, HCHIB |
| | | | GACACTGCCGCAA | | | | | | basic chitinase \| |
| | | | GGAAGAAAGAGCT | | | | | | chr3: |
| | | | CTCTGCC (SEQ ID | | | | | | 3962382- |
| | | | NO: | | | | | | 3963984 REVERSE |
| | | | 99) | | | | | | LENGTH = 1603 |
| 24,717,963 | A | T | AATCGCAAATGTTT | ATG | M | TV | fgenesh | AT3G119 | \| Symbols: |
| | | | CCTGTCACAGGGAC | -> | -> K | | masked- | 80.1 | MS2, FAR2 \| |
| | | | AAGCTTGTCTAACA | AAG | | | N1- | | Jojoba acyl CoA |
| | | | TGAAAGAC[A/T]TG | | | | abinit- | | reductase- |
| | | | AAAGATGCTCCATG | | | | gene- | | related male |
| | | | AGTCTCTCTTAGAT | | | | 24.356 | | sterility |
| | | | TTTTAAAAAGCTCT | | | | | | protein \| chr3: |
| | | | GCATCT (SEQ ID | | | | | | 3814236- |
| | | | NO: | | | | | | 3817117 FORWARD |
| | | | 100) | | | | | | LENGTH = 2882 |
| 24,717,966 | A | T | CGCAAATGTTTCCT | TTC | F | TV | fgenesh | AT3G119 | \| Symbols: |
| | | | GTCACAGGGACAA | -> | -> Y | | masked- | 80.1 | MS2, FAR2 \| |
| | | | GCTTGTCTAACATG | TAC | | | N1- | | Jojoba acyl CoA |
| | | | AAAGACATG[A/T]A | | | | abinit- | | reductase- |
| | | | AGATGCTCCATGAG | | | | gene- | | related male |
| | | | TCTCTCTTAGATTTT | | | | 24.356 | | sterility |
| | | | TAAAAAGCTCTGCA | | | | | | protein \| chr3: |
| | | | TCTAAC (SEQ ID | | | | | | 3814236- |
| | | | NO: 101) | | | | | | 3817117 FORWARD |
| | | | | | | | | | LENGTH = 2882 |

TABLE 13
SNPs in chromosome N6 distributed across the QTL3 interval

| Position | Ref Nt | Alt Nt | Sequence | Codon Change | AA Change | Type | Affected gene | Ortholog | Description |
|---|---|---|---|---|---|---|---|---|---|
| 19,027,546 | G | A | TATCTTATTAAAATA | None | None | TS | None | None | None |
| | | | GAAATACATTTAAA | | | | | | |
| | | | GAATATTTGGAAAC | | | | | | |
| | | | ATAAATA[G/A]CAGT | | | | | | |
| | | | AAAAAAATATAATAT | | | | | | |
| | | | TATTTGAAAACATAG | | | | | | |
| | | | ATATCAATATATTAA | | | | | | |
| | | | A (SEQ ID NO: 102) | | | | | | |
| 19,085,314 | C | T | CTTAGACAAAGACTC | GGT −> AGT | G −> S | TS | maker- | #N/A | #N/A |
| | | | AGCGGACTCTTTGTG | | | | N6- | | |
| | | | CACAATAGTAACGG | | | | augustus- | | |
| | | | CATCAC[C/T]GTCTT | | | | gene- | | |
| | | | CAGGCACAGGAAGA | | | | 19.60 | | |
| | | | GACGAAATGGTTTC | | | | | | |
| | | | AGATTCCCTGGGAG | | | | | | |
| | | | GAC (SEQ ID NO: 103) | | | | | | |
| 19,156,645 | G | T | TTCAAGTCCCTAACC | None | None | TV | maker- | AT5G24740.1 | \| Symbols: \| Protein of unknown function |
| | | | TCTCTCAAAGCCATA | | | | N6- | | (DUF1162) \| chr5: 8469951-8489703 |
| | | | AAAGCTCTTAACTAG | | | | augustus- | | REVERSE LENGTH = 19753 |
| | | | AGAAT[G/T]AATTCA | | | | gene- | | |
| | | | AAGACCAATAGTAT | | | | 19.65 | | |
| | | | GTTCTTCATTCCCAC | | | | | | |
| | | | TATTTATAGCCTATC | | | | | | |
| | | | (SEQ ID NO: 104) | | | | | | |
| 19,199,109 | G | A | GATCATGTTCCGTTT | CGG −> CGA | None | TS | maker- | AT5G24830.1 | \| Symbols: \| Tetratricopeptide repeat |
| | | | TGGATACTTATGAG | | | | N6- | | (TPR)-like superfamily protein \| |
| | | | GAAATACACCAAGT | | | | augustus- | | chr5: 8530978-8533867 FORWARD |
| | | | TGAGGCG[G/A]CTA | | | | gene- | | LENGTH = 2890 |
| | | | GACGAGGCGTATTT | | | | 19.10 | | |
| | | | AGCTTACAAAAAAT | | | | | | |
| | | | GGCTAATGACGAGG | | | | | | |
| | | | AGCAG (SEQ ID NO: 105) | | | | | | |
| 19,325,186 | G | C | GCGTTGAAAAACTTC | GAC −> GAG | D −> E | TV | maker- | AT5G10930.1 | \| Symbols: CIPK5, SnRK3.24 \| CBL- |
| | | | GGCGACACCACCAC | | | | N6- | | interacting protein kinase 5 \| |
| | | | CGCCGGTACCGCCG | | | | fgenesh- | | chr5: 3445366-3447114 REVERSE |
| | | | CTCCGTC[G/C]TCCT | | | | gene- | | LENGTH = 1749 |
| | | | CCTCCTCGCCGTCGT | | | | 19.350 | | |
| | | | TAACGACCGTGATC | | | | | | |
| | | | ATTGGAGCCTTGAA | | | | | | |
| | | | GCT (SEQ ID NO: 106) | | | | | | |
| 19,402,086 | T | C | TTAAGCTCAAGAAA | TTC −> CTC | F −> L | TS | augustuS_ | #N/A | #N/A |
| | | | GCCAAAGTCAGACC | | | | masked- | | |
| | | | TGTTCTGTTCTCGTG | | | | N6- | | |
| | | | TCCGACG[T/C]TCCG | | | | abinit- | | |
| | | | GTGGGGAAACCGGC | | | | gene- | | |
| | | | AGCCACGGTCGAGA | | | | 19.165 | | |
| | | | AAAGAAGCTGAATC | | | | | | |
| | | | AGCG (SEQ ID NO: 107) | | | | | | |
| 19,513,420 | A | C | TATATATAGGTGGAT | ATT −> CTT | I−> L | TV | maker- | AT5G25430.1 | \| Symbols: \| HCO3-transporter family \| |
| | | | CTCTGAGTGCTGTG | | | | N6- | | chr5: 8851251-8854259 FORWARD |
| | | | GAAACATTAGCTTCT | | | | augustus- | | LENGTH = 3009 |
| | | | ACTTCT[A/C]TTTGC | | | | gene- | | |
| | | | GGAATCATCCACGC | | | | 19.27 | | |
| | | | CATCTTTGGTGGACA | | | | | | |
| | | | GCCATTGTTGATACT | | | | | | |
| | | | T (SEQ ID NO: 108) | | | | | | |
| 19,583,431 | T | C | TGTTTTAATAGGACG | None | None | TS | None | None | None |
| | | | AGCTATGGGAAGCC | | | | | | |
| | | | TGTTTAACAATGATG | | | | | | |
| | | | GGCCTG[T/C]CTGCA | | | | | | |
| | | | AACCCTGAAACGTCT | | | | | | |
| | | | CCCCAGAATTTAGCA | | | | | | |
| | | | TTGTACAAACTTTAA | | | | | | |
| | | | (SEQ ID NO: 109) | | | | | | |
| 19,601,021 | G | A | TCCGAAATACCCAAA | None | None | TS | None | None | None |
| | | | AATACCTAATCTAAA | | | | | | |
| | | | CAAAATATTTCGCAT | | | | | | |
| | | | ACTTT[G/A]GGTACC | | | | | | |
| | | | CGATCGGGTCTCCG | | | | | | |
| | | | GTAAGATCCAGACC | | | | | | |
| | | | CAAACCGAGATCGT | | | | | | |
| | | | AT (SEQ ID NO: 110) | | | | | | |
| 19,706,563 | C | T | TACATGCTAACAGTG | GAG −> GAA | None | TS | maker- | AT5G25950.1 | \| Symbols: \| Protein of Unknown Function |
| | | | ATGTGATATTGTTCA | | | | N6- | | (DUF239) \| chr5: 9057793-9059963 |
| | | | CTCGAGGGAGTTGA | | | | augustus- | | REVERSE LENGTH = 2171 |
| | | | AACGGG[C/T]TCTAC | | | | gene- | | |
| | | | GGCTGCACCCAGGG | | | | 19.94 | | |
| | | | CGAATCTAGTTGTTG | | | | | | |
| | | | TTTGGACAAAACCG | | | | | | |
| | | | GA (SEQ ID NO: 111) | | | | | | |
| 19,800,643 | C | T | GATGCTTCCACCGAC | GGC −> GGT | None | TS | augustus_ | AT5G40890.1 | \| Symbols: ATCLC-A, CLC-A, CLCA, ATCLCA |
| | | | GGCGTTGGCTTACTC | | | | mask | | \| chloride channel A \| chr5: 16381346- |
| | | | AGCTCCCCCCGTGAC | | | | ed-N6- | | 16385319 REVERSE LENGTH = 3974 |
| | | | GGCGG[C/T]GGTGG | | | | abinit- | | |
| | | | CGTTGATAGTCTTGA | | | | gene- | | |
| | | | TTACGAGGTTATCGA | | | | 19.239 | | |
| | | | GAATTACGCTTACAG | | | | | | |
| | | | (SEQ ID NO: 112) | | | | | | |
| 19,906,666 | A | C | CCGTGTGGTGGTAG | GAA −> GAC | E −> D | TV | maker- | #N/A | #N/A |
| | | | AATCATCGACGAGG | | | | N6- | | |
| | | | TTTGCGTCAAGGAG | | | | augustus- | | |
| | | | GAGTACGA[A/C]ACT | | | | gene- | | |
| | | | CGTCCCGGGAAGCG | | | | 19.53 | | |
| | | | CTTCTTCAGCTGCAT | | | | | | |
| | | | AAACTACGAGGTAA | | | | | | |
| | | | CCCA (SEQ ID NO: 113) | | | | | | |
| 20,000,119 | A | G | TATAAAGCCAGCGG | TAC −> TGC | Y −> C | TS | maker- | AT5G26810.1 | \| Symbols: \| Pectin lyase-like superfamily |
| | | | TAGCGCTTACGGTTT | | | | N6- | | protein \| chr5: 9430952-9432969 |
| | | | ATGGCGATAAATCA | | | | fgenesh- | | FORWARD LENGTH = 2018 |
| | | | GCGTTCT[A/G]CAAC | | | | gene- | | |
| | | | TGCGGTTTCTTGGG | | | | 20.335 | | |
| | | | GTTACAAGATACGTT | | | | | | |
| | | | GTGGGATGTTCAAG | | | | | | |
| | | | GCA (SEQ ID NO: 114) | | | | | | |
| 20,095,002 | C | T | AGGTCTTCTGCCAAA | GTG −> ATG | V −> M | TS | maker- | AT2G01390.1 | \| Symbols: \| Tetratricopeptide repeat |
| | | | CCCAAGCCTGTGGA | | | | N6- | | (TPR)-like superfamily protein \| |
| | | | TCAACGTTGCTCCTT | | | | augustus- | | chr2: 172256-174137 FORWARD |
| | | | GGTACA[C/T]ACCAA | | | | gene- | | LENGTH = 1882 |
| | | | GTGAGTGATGGGCT | | | | 20.62 | | |
| | | | TTCACCATCTCCTTTA | | | | | | |
| | | | CTGCTTCGATAAGTT | | | | | | |
| | | | (SEQ ID NO: 115) | | | | | | |
| 20,205,211 | G | A | CAATAATGTGAACTC | CTG −> TTG | None | TS | augustus_ | AT5G27470.1 | \| Symbols: \| seryl-tRNA synthetase / |
| | | | TCTTTTCTTCAAGAA | | | | mask | | serine-tRNA ligase \| chr5: 9695008- |
| | | | GTCTAGCCCAAACCT | | | | ed-N6- | | 9697389 FORWARD LENGTH = 2382 |
| | | | GATCA[G/A]AGCCT | | | | abinit- | | |
| | | | GGTTAAGTAGCACA | | | | gene- | | |
| | | | CCATCTCCTTTAAGG | | | | 20.166 | | |
| | | | AAAAAGCCTCTTCCT | | | | | | |
| | | | C (SEQ ID NO: 116) | | | | | | |
| 20,300,571 | G | A | CGTCGATAGTTCACA | CGC −> CGT | None | TS | maker- | AT5G27320.1 | \| Symbols: ATGID1C, GID1C \| alpha/beta- |
| | | | GAGACAACAACGAA | | | | N6- | | Hydrolases superfamily protein \| |
| | | | ACCGCACAGACCAA | | | | fgenesh- | | |
| | | | CAAGCCT[G/A]CGG | | | | gene- | | chr5: 9629087-9631210 FORWARD |
| | | | CAAAGAGTGTCATA | | | | 20.419 | | LENGTH = 2124 |
| | | | GATAGCACTGTTTGC | | | | | | |
| | | | AGAAGAGTGCACAA | | | | | | |
| | | | AGCT (SEQ ID NO: 117) | | | | | | |
| 20,406,148 | C | T | GTTACAGATACGAG | CTA −> TTA | None | TS | augustus_ | AT5G28210.1 | \| Symbols: \| mRNA capping enzyme family |
| | | | ATGGAGCCGTTTGG | | | | mask | | protein \| chr5: 10188586-10190463 |
| | | | TGTACGGTTAAAGG | | | | ed-N6- | | FORWARD LENGTH = 1878 |
| | | | CCTTTTGC[C/T]TACT | | | | abinit- | | |
| | | | CTCTTCCGTGGAGAA | | | | gene- | | |
| | | | GAAGGTGTTCAACG | | | | 20.212 | | |
| | | | AGCTGATACCTTCGC | | | | | | |
| | | | TC (SEQ ID NO: 118) | | | | | | |
| 20,407,023 | G | T | TAATCCCTTAGATTC | None | None | TV | None | None | None |
| | | | AAGCAGCAACGCCT | | | | | | |
| | | | GTAGGCTCCCTGAG | | | | | | |
| | | | ATTCATT[G/T]CCCA | | | | | | |
| | | | TTATGTGTCTTAGCC | | | | | | |
| | | | ATCAATCTAATTGCA | | | | | | |
| | | | TAAACTAGTAAGAA | | | | | | |
| | | | CA (SEQ ID NO: 119) | | | | | | |
| 20,505,840 | A | G | GCCGGTCTAGCACA | GAT −> GAC | None | TS | maker- | AT5G28680.1 | \| Symbols: ANX2 \| Malectin/receptor-like |
| | | | TAAGATCTCAAACAA | | | | N6- | | protein kinase family protein |
| | | | AACAACTCCAAAAG | | | | augustus- | | chr5: 10719437-10722013 REVERSE |
| | | | AGTACAC[A/G]TCTG | | | | gene- | | LENGTH = 2577 |
| | | | ACTTCTCGGTTAACT | | | | 20.93 | | |
| | | | GCTGTCTCCTAAAGT | | | | | | |
| | | | ACTCTGGATCTAAGT | | | | | | |
| | | | A (SEQ ID NO: 120) | | | | | | |
| 20,601,198 | G | A | TGGTTGGACTGTGC | ACG −> ACA | None | TS | fgenesh_ | AT5G49580.1 | \| Symbols: \| Chaperone DnaJ-domain |
| | | | AATCCGAGGCTTTG | | | | masked- | | superfamily protein \| chr5: 20123823- |
| | | | ACTCTTTCATCAGGA | | | | N6- | | 20126813 REVERSE LENGTH = 2991 |
| | | | TGGGGAC[G/A]GCA | | | | abinit- | | |
| | | | TCGTTTTTCTCAATC | | | | gene- | | |
| | | | ATGTGGTGTGGTGT | | | | 20.524 | | |
| | | | CTTCTCAGCGTTTTC | | | | | | |
| | | | CAT (SEQ ID NO: 121) | | | | | | |
| 20,631,917 | T | C | CTTGGATAAGAGTTT | None | None | TS | None | None | None |
| | | | ATAGGAAAACCAAA | | | | | | |
| | | | ATTCGCTTGCAATGA | | | | | | |
| | | | GTATCA[T/C]TATTA | | | | | | |
| | | | AACCGATCATGAGG | | | | | | |
| | | | AGATCATAACAACTA | | | | | | |
| | | | GAATTTAAATGATGT | | | | | | |
| | | | T (SEQ ID NO: 122) | | | | | | |
| 20,702,631 | T | C | TTGTGTAGATGGTC | AAT −> AAC | None | TS | maker- | AT5G49330.1 | \| Symbols: MYB111, ATMYB111, PFG3 \| |
| | | | GCATATTGCAACACA | | | | N6- | | myb domain protein 111 \| |
| | | | TCTACCAGGAAGAA | | | | augustus- | | chr5: 19998952-20001378 REVERSE |
| | | | CAGACAA[T/C]GAA | | | | gene- | | LENGTH = 2427 |
| | | | ATTAAAAACTATTGG | | | | 20.37 | | |
| | | | AATTCACATCTCAGC | | | | | | |
| | | | CGCAAAATCTATGCC | | | | | | |
| | | | TT (SEQ ID NO: 123) | | | | | | |
| 20,805,841 | G | A | ACAGGGTATACCTG | CTC −> CTT | None | TS | maker- | AT3G06930.2 | \| Symbols: ATPRMT4B, PRMT4B \| protein |
| | | | TGGCTGAGACATTCT | | | | N6- | | arginine methyltransferase 4B \| |
| | | | ATAATAAGGCTCCTT | | | | augustus- | | chr3: 2185143-2189387 REVERSE |
| | | | CAAGTC[G/A]AGTTT | | | | gene- | | LENGTH = 4245 |
| | | | GCACGATGATGTTT | | | | 20.112 | | |
| | | | GGAGGATTCCACCTT | | | | | | |
| | | | GATTGGCACCGGGC | | | | | | |
| | | | CC (SEQ ID NO: 124) | | | | | | |
| 20,816,431 | C | A | CTCCCTACTGATTTC | None | None | TV | None | None | None |
| | | | TCCAGGTTTAAGAC | | | | | | |
| | | | GATGCACATGTACG | | | | | | |
| | | | ATATCGT[C/A]GTCA | | | | | | |
| | | | AGAACCGCAACATG | | | | | | |
| | | | TTCCAAGTAAGGGA | | | | | | |
| | | | TATGTAGCCATATTT | | | | | | |
| | | | TGG (SEQ ID NO: 125) | | | | | | |
| 20,846,412 | C | A | AAACGAGATGATTTT | None | None | TV | None | None | None |
| | | | CCTTTAAACGTTTAA | | | | | | |
| | | | AAAAAATCAATCTA | | | | | | |
| | | | GGCATT[C/A]AAAAA | | | | | | |
| | | | ATCGGTCTGCCAATA | | | | | | |
| | | | CTATTAGCTATTTTCT | | | | | | |
| | | | GAACTTTGGTTGCT | | | | | | |
| | | | (SEQ ID NO: 126) | | | | | | |
| 20,902,530 | G | A | GCAGCGGTTCGCTC | TCC −> TCT | None | TS | augustus_ | AT5G48890.1 | \| Symbols: \| C2H2-like zinc finger protein |
| | | | CTTCTTGTGGGCGTT | | | | masked- | | \| chr5: 19820353-19820874 FORWARD |
| | | | CTGGTGGCCTCCTAA | | | | N6- | | LENGTH = 522 |
| | | | GGCTTG[G/A]GAAC | | | | abinit- | | |
| | | | TCTGGAACTTTCTAG | | | | gene- | | |
| | | | AACAAAAGAGGCAA | | | | 20.311 | | |
| | | | GAGAATACTCTTGAT | | | | | | |
| | | | GA (SEQ ID NO: 127) | | | | | | |
| 21,001,046 | G | A | TTTAGGAAGATTAG | AAC −> AAT | None | TS | maker- | #N/A | #N/A |
| | | | ACAGAACATACCACT | | | | N6- | | |
| | | | GTCAGTAGCTTTGAC | | | | fgenesh- | | |
| | | | GATGGC[G/A]TTACT | | | | gene- | | |
| | | | GATAAGAGGCACTT | | | | 21.260 | | |
| | | | TAGAACACATGGCA | | | | | | |
| | | | GCAACTACCTGCATG | | | | | | |
| | | | CT (SEQ ID NO: 128) | | | | | | |
| 21,100,769 | G | A | TCCCTCTGTCAAAAC | TCG −> TCA | None | TS | maker- | AT5G48250.1 | \| Symbols: \| B-box type zinc finger protein |
| | | | TGTGACTGGTTAGG | | | | N6- | | with CCT domain \| chr5: 19561319- |
| | | | CCACAACGGAGCTA | | | | fgenesh- | | 19563722 REVERSE LENGTH = 2404 |
| | | | CTAATTC[G/A]CATC | | | | gene- | | |
| | | | ATAAGAAGCAAACC | | | | 21.198 | | |
| | | | ATTAACTGCTACTCT | | | | | | |
| | | | GGTTGCCCCTCGAGT | | | | | | |
| | | | GA (SEQ ID NO: 129) | | | | | | |
| 21,199,766 | G | A | CTGTGATAAACGATC | CTG −> TTG | None | TS | augustus_ | AT5G47840.1 | \| Symbols: AMK2 \| adenosine |
| | | | TTTGACCATCATTAC | | | | masked- | | monophosphate kinase \| chr5: 19375441- |
| | | | AACTATTTCATTAGG | | | | N6- | | 19378339 FORWARD LENGTH = 2899 |
| | | | GACCA[G/A]TTGTCC | | | | abinit- | | |
| | | | TTTCTCCATGTGTTCT | | | | gene- | | |
| | | | TTTGCCAGTCTTCCA | | | | 21.377 | | |
| | | | TTCTCACTCCCAG | | | | | | |
| | | | (SEQ ID NO: 130) | | | | | | |
| 21,218,604 | G | T | GGAACTATTTTCTAA | None | None | TV | None | None | None |
| | | | GTGGATCAACTCAA | | | | | | |
| | | | ACGACGCCGTTTCGC | | | | | | |
| | | | TTCTAT[G/T]TAAGT | | | | | | |
| | | | CAAAAGTCCTCTCTT | | | | | | |
| | | | TTATGTTTTGTATCC | | | | | | |
| | | | AAAGTATACAGCTTT | | | | | | |
| | | | (SEQ ID NO: 131) | | | | | | |

TABLE 14
SNPs in chromosome N6 that are in candidate genes within the QTL3 interval

| | Ref | Alt | | Codon | | | | | |
|---|---|---|---|---|---|---|---|---|---|
| Position | Nt | Nt | Sequence | Change | AA Change | Type | Affected gene | Ortholog | Description |
| 19,336,744 | A | G | GAGTTTTGCAGCAGCAGATTACT | AAG −> | K −> R | TS | maker-N6- | AT5G25120.1 | \| Symbols: CYP71B11 \| ytochrome p450, |
| | | | TCCCGGTAATTGGTAAATTAATCG | AGG | | | fgenesh-gene- | | family 71, subfamily B, polypeptide 11 \| |
| | | | ATA[A/G]GATCACAGGGTTACAT | | | | 19.294 | | chr5: 8662099-8664533 FORWARD |
| | | | AGCAAATGTGAGAAGGTTTTTAA | | | | | | LENGTH = 2435 |
| | | | AGCAATGGATG (SEQ ID NO: 132) | | | | | | |
| 19,336,819 | G | A | TGAGAAGGTTTTTAAAGCAATGG | CGT −> | R −> H | TS | maker-N6- | AT5G25120.1 | \| Symbols: CYP71B11 \| ytochrome p450, |
| | | | ATGCATTTTTCGATCAATCTATAA | CAT | | | fgenesh-gene- | | family 71, subfamily B, polypeptide 11 \| |
| | | | AGC[G/A]TCATCTTGAAGACGAG | | | | 19.294 | | chr5: 8662099-8664533 FORWARD |
| | | | AGCCTTGAAGATGATATCATAGC | | | | | | LENGTH = 2435 |
| | | | CTTGCTCTTAA (SEQ ID NO: 133) | | | | | | |
| 19,337,615 | A | C | TTGATCTTGAAGAATCATATGGA | AAG −> | K −> Q | TV | maker-N6- | AT5G25120.1 | \| Symbols: CYP71B11 \| ytochrome p450, |
| | | | CTCGTATGTCCTAAGAAAGTTCCA | CAG | | | fgenesh-gene- | | family 71, subfamily B, polypeptide 11 \| |
| | | | CTT[A/C]AGCTTATCCCGATTCTTA | | | | 19.294 | | chr5: 8662099-8664533 FORWARD |
| | | | CTCAATGGACTTGACTTCGATTTA | | | | | | LENGTH = 2435 |
| | | | TATTTTCG (SEQ ID NO: 134) | | | | | | |
| 19,350,156 | A | C | ACTGAAACTTCCTATAATTGGCAA | CAT −> | H −> P | TV | maker-N6- | AT5G25130.1 | \| Symbols: CYP71B12 \| cytochrome P450, |
| | | | CTTGCACCAATTAGGCTCGCAGC | CCT | | | fgenesh-gene- | | family 71, subfamily B, polypeptide 12 \| |
| | | | CTC[A/C]CGTTCATTAACAAAAT | | | | 19.295 | | chr5: 8668299-8670194 FORWARD |
| | | | TATCTGAAAAGTATGGACCTCTA | | | | | | LENGTH = 1896 |
| | | | ATGTCCCTAA (SEQ ID NO: 135) | | | | | | |
| 19,353,584 | C | T | GTTGTATGAAAGTCTTGCAGCGT | GAT −> | D −> N | TS | maker-N6- | AT5G25140.1 | \| Symbols: CYP71B13 \| cytochrome P450, |
| | | | ACGTCAAGTAAGGTCGTGAACAA | AAT | | | augustus-gene- | | family 71, subfamily B, polypeptide 13 \| |
| | | | CAAT[C/T]AACGTCGAACGTTTTC | | | | 19.75 | | chr5: 8672424-8674629 FORWARD |
| | | | AAGACATCTTTCACTGTCTCTGGA | | | | | | LENGTH = 2206 |
| | | | GTTGACGCCA (SEQ ID NO: 136) | | | | | | |
| 19,353,648 | A | C | TTCAAGACATCTTTCACTGTCTCT | AAT −> | N −> K | TV | maker-N6- | AT5G25140.1 | \| Symbols: CYP71B13 \| cytochrome P450, |
| | | | GGAGTTGACGCCACAACGGTAGA | AAG | | | augustus-gene- | | family 71, subfamily B, polypeptide 13 \| |
| | | | CAC[A/C]TTTCCAAGCTTGAGGGA | | | | 19.75 | | chr5: 8672424-8674629 FORWARD |
| | | | CATTAGAGCTCCATATTTTTCAGA | | | | | | LENGTH = 2206 |
| | | | TAATTTGAA (SEQ ID NO: 137) | | | | | | |
| 19,353,749 | G | T | CATGGAACGATGAGGTTTGGATC | CTT −> | L −> 1 | TV | maker-N6- | AT5G25140.1 | \| Symbols: CYP71B13 \| cytochrome P450, |
| | | | CCAGTTGGTGCAAGTTACCAATT | ATT | | | augustus-gene- | | family 71, subfamily B, polypeptide 13 \| |
| | | | ATAA[G/T]AAGCCTTGGTGGTCC | | | | 19.75 | | chr5: 8672424-8674629 FORWARD |
| | | | AGGAGGTAGGTTTTTCTTTGTCTT | | | | | | LENGTH = 2206 |
| | | | TCTCGTGTTCT (SEQ ID NO: 138) | | | | | | |
| 19,476,836 | A | C | CTACATATGCATAGTTTCCATACA | None | None | TV | maker-N6- | AT1G52570.1 | \| Symbols: PLDALPHA2 \| phospholipase D |
| | | | GAGAGATAGATAGCAGTAGGTAT | | | | augustus-gene- | | alpha 2 \| chr1: 19583940-19587050 |
| | | | AAG[A/C]TAAGCTAAGCTAACCT | | | | 19.80 | | REVERSE LENGTH = 3111 |
| | | | ACAGATATAATGAAACAATTACA | | | | | | |
| | | | AGATGTAAAAA (SEQ ID NO: 139) | | | | | | |
| 19,783,834 | G | C | TTTAGCTGTGTTCCTCACCTCATTC | CAG −> | Q −> E | TV | augustus_maske | AT5G26220.1 | \| Symbols: \| ChaC-like family protein \| |
| | | | GCCAATTCTATCACGAACTCTTCC | GAG | | | d-N6-abinit- | | chr5: 9162969-9164685 REVERSE |
| | | | T[G/C]ATGCTCTGTTTTGACAACA | | | | gene-19.236 | | LENGTH = 1717 |
| | | | TTCATCAAACAAATCAACGCCAAA | | | | | | |
| | | | AATCTTA (SEQ ID NO: 140) | | | | | | |
| 19,784,007 | T | C | TTGAAAAGGTACTCCCGGTTGTTC | AAA −> | K −> R | TS | augustus_maske | AT5G26220.1 | \| Symbols: \| ChaC-like family protein \| |
| | | | CCACAAGGTTCAGAAGCTGTGGC | AGA | | | d-N6-abinit- | | chr5: 9162969-9164685 REVERSE |
| | | | GAT[T/C]TGCATGGCCATCTCCTC | | | | gene-19.236 | | LENGTH = 1717 |
| | | | CAAAAGTAGTATTTGTTCGACACT | | | | | | |
| | | | TTGTCTGGT (SEQ ID NO: 141) | | | | | | |
| 19,784,367 | T | C | TCGACAAGGGTTTTAGAGTCGTA | ATA −> | I −> M | TS | augustus_maske | AT5G26220.1 | \| Symbols: \| ChaC-like family protein \| |
| | | | TTCACACTCTCTTCGTTCCAAGTA | ATG | | | d-N6-abinit- | | chr5: 9162969-9164685 REVERSE |
| | | | CTC[T/C]ATGGCTAGCTTCTCCTTC | | | | gene-19.236 | | LENGTH = 1717 |
| | | | TCAGGTCCTCCATGAACACAATAA | | | | | | |
| | | | GCAGCACC (SEQ ID NO: 142) | | | | | | |
| 19,784,633 | A | G | CAACATTGATCTATTTCTTTGTTG | TTT −> | F −> L | TS | augustus_maske | AT5G26220.1 | \| Symbols: \| ChaC-like family protein \| |
| | | | ATAATTCAAGAACAAGTGTGTTG | CTT | | | d-N6-abinit- | | chr5: 9162969-9164685 REVERSE |
| | | | TAA[A/G]CATACCGAGATCAAAG | | | | gene-19.236 | | LENGTH = 1717 |
| | | | ACGCGTTTGTAGTCTTTAATATTG | | | | | | |
| | | | CCAATGAGTT (SEQ ID NO: 143) | | | | | | |
| 19,784,672 | T | A | GTGTGTTGTAAACATACCGAGAT | AAT −> | N −> Y | TV | augustus_maske | AT5G26220.1 | \| Symbols: \| ChaC-like family protein \| |
| | | | CAAAGACGCGTTTGTAGTCTTTAA | TAT | | | d-N6-abinit- | | chr5: 9162969-9164685 REVERSE |
| | | | TAT[T/A]GCCAATGAGTTTTTCGT | | | | gene-19.236 | | LENGTH = 1717 |
| | | | CAAAATCGAAACCTGGGTTCCAT | | | | | | |
| | | | ATTATAGATC (SEQ ID NO: 144) | | | | | | |
| 19,784,688 | G | T | CCGAGATCAAAGACGCGTTTGTA | GAC −> | D −> E | TV | augustus_maske | AT5G26220.1 | \| Symbols: \| ChaC-like family protein \| |
| | | | GTCTTTAATATTGCCAATGAGTTT | GAA | | | d-N6-abinit- | | chr5: 9162969-9164685 REVERSE |
| | | | TTC[G/T]TCAAAATCGAAACCTGG | | | | gene-19.236 | | LENGTH = 1717 |
| | | | GTTCCATATTATAGATCCATATCC | | | | | | |
| | | | GAATTCCCA (SEQ ID NO: 145) | | | | | | |
| 19,784,733 | TT | CA | TTTTCGTCAAAATCGAAACCTGG | GAA −> | E −> V | Subst | augustus_maske | AT5G26220.1 | \| Symbols: \| ChaC-like family protein \| |
| | | | GTTCCATATTATAGATCCATATCC | GTG | | | d-N6-abinit- | | chr5: 9162969-9164685 REVERSE |
| | | | GAA[TT/CA]CCCACAATACCATCT | | | | gene-19.236 | | LENGTH = 1717 |
| | | | TGAACGTTAAAGTCTCTTTTAGGA | | | | | | |
| | | | AGAAGAAATAG (SEQ ID NO: 146) | | | | | | |
| 19,800,525 | A | T | CCTAAATCAAGCAAGCTTCTCGAT | CAC −> | H −> L | TV | augustus_maske | AT5G40890.1 | \| Symbols: ATCLC-A, CLC-A, CLCA, ATCLCA |
| | | | TCGATTCGACTCGATCATCATCAT | CTC | | | d-N6-abinit- | | \| chloride channel A \| chr5: 16381346- |
| | | | GC[A/T]CTCGAATCATCTACAGAA | | | | gene-19.239 | | 16385319 REVERSE LENGTH = 3974 |
| | | | CGGGATCGAATCCGATAATCTCC | | | | | | |
| | | | TCTGGTCTC (SEQ ID NO: 147) | | | | | | |
| 20,191,826 | G | A | ACTTAATTCACTTTTTAAAACTGTT | ATC −> | None | TS | augustus_maske | AT5G27600.1 | \| Symbols: LACS7, ATLACS7 \| long-chain |
| | | | ACTATTATCACACACTACCATTTA | ATT | | | d-N6-abinit- | | acyl-CoA synthetase 7 \| chr5: 9742576- |
| | | | A[G/A]ATTGCCTTCTTCTTTGAGT | | | | gene-20.163 | | 9747005 FORWARD LENGTH = 4430 |
| | | | TATAGGCAAGTTGGAAAAGCCTT | | | | | | |
| | | | CTTTTTGTA (SEQ ID NO: 148) | | | | | | |
| 20,300,548 | A | G | AAGGGTAACGATTCTCCGGCGCA | TTC −> | F −> S | TS | maker-N6- | AT5G27320.1 | \| Symbols: ATGID1C, GID1C \| alpha/beta- |
| | | | CGTCGATAGTTCACAGAGACAAC | TCC | | | fgenesh-gene- | | Hydrolases superfamily protein \| |
| | | | AACG[A/G]AACCGCACAGACCAA | | | | 20.419 | | chr5: 9629087-9631210 FORWARD |
| | | | CAAGCCTGCGGCAAAGAGTGTCA | | | | | | LENGTH = 2124 |
| | | | TAGATAGCACTG (SEQ ID NO: 149) | | | | | | |
| 20,375,643 | T | A | GATTCTAGCATTTTACTCACAGTG | GAA −> | E −> D | TV | maker-N6- | AT5G27980.1 | \| Symbols: \| Seed maturation protein \| |
| | | | AGAACATCGGCAAGCGTAGTCTT | GAT | | | augustus-gene- | | chr5: 10015774-10016726 REVERSE |
| | | | TTC[T/A]TCATCGGAGTTAGCTCG | | | | 20.83 | | LENGTH = 953 |
| | | | AGCGTTAAGAGTGGCAGCTGACT | | | | | | |
| | | | GAGCAGAAGC (SEQ ID NO: 150) | | | | | | |
| 20,766,637 | G | A | TTCTGCCTCTTGAGAGAAAATCAT | CGT −> | R −> H | TS | maker-N6- | AT4G02280.1 | \| Symbols: SUS3, ATSUS3 \| sucrose |
| | | | GCCAACGGGTAGGTTCGAGACG | CAT | | | augustus-gene- | | synthase 3 \| chr4: 994927-998967 |
| | | | ATGC[G/A]TGAATGGGTTCACGA | | | | 20.39 | | FORWARD LENGTH = 4041 |
| | | | CGCCATCTCTGCTCAACGCAATGA | | | | | | |
| | | | GCTCCTCTCTC (SEQ ID NO: 151) | | | | | | |
| 20,769,461 | A | C | TATAGTCTCTCCAGGAGCTGATAT | AAG −> | K −> T | TV | maker-N6- | AT4G02280.1 | \| Symbols: SUS3, ATSUS3 \| sucrose |
| | | | GACCATATACTTTCCTTATTCTGA | ACG | | | augustus-gene- | | synthase 3 \| chr4: 994927-998967 |
| | | | CA[A/C]GGAAAGAAGACTAACTG | | | | 20.39 | | FORWARD LENGTH = 4041 |
| | | | CCCTTCATGAGTCTATTGAAGAAC | | | | | | |
| | | | TTCTGTTTA (SEQ ID NO: 152) | | | | | | |
| 20,770,769 | A | G | ATGATAATTTTTTTCTGGTTTTGC | AAT −> | N −> D | TS | maker-N6- | AT4G02280.1 | \| Symbols: SUS3, ATSUS3 \| sucrose |
| | | | AGGCCAATTCAATCCCACTGGCA | GAT | | | augustus-gene- | | synthase 3 \| chr4: 994927-998967 |
| | | | ACT[A/G]ATGAGCATTGAGCAAG | | | | 20.39 | | FORWARD LENGTH = 4041 |
| | | | CTATGGTTGGATTCTAATACTTGC | | | | | | |
| | | | TGCACTCCCT (SEQ ID NO: 153) | | | | | | |
| 20,823,998 | CGC | TGT | TACAAATTTGGCTCAAGTCTGGG | GCG −> | A −> T | Substi- | maker-N6- | AT5G48960.1 | \| Symbols: \| HAD-superfamily hydrolase, |
| | | | CACAAACAACAGCTAGTTTTCCAT | ACA | | tution | augustus-gene- | | subfamily IG, 5′-nucleotidase \| |
| | | | AGC[CGC/TGT]ATCAGGAAGGGG | | | | 20.116 | | chr5: 19849543-19853564 FORWARD |
| | | | AGAATCATGAGCCAGTGACTGCA | | | | | | LENGTH = 4022 |
| | | | GAAGAGAGATACAAC (SEQ ID NO: 154) | | | | | | |
| 20,825,959 | T | C | ATGAAATTTAGGTCCTGTGAAGC | AAT −> | N −> D | TS | maker-N6- | AT5G48960.1 | \| Symbols: \| HAD-superfamily hydrolase, |
| | | | AAATGACCTTGTACAGTCCTTTGT | GAT | | | augustus-gene- | | subfamily IG, 5′-nucleotidase |
| | | | AAT[T/C]AAGAGTGCCAAGATCT | | | | 20.116 | | chr5: 19849543-19853564 FORWARD |
| | | | GCTGAAATAGATCCATCATCCAAT | | | | | | LENGTH = 4022 |
| | | | CTATCAACCA (SEQ ID NO: 155) | | | | | | |
| 20,826,301 | G | C | TGAATGGTGTTGAAAAGTTTACC | AAC −> | N −> K | TV | maker-N6- | AT5G48960.1 | \| Symbols: \| HAD-superfamily hydrolase, |
| | | | ATACCTGACAGCTTTATTTGATAA | AAG | | | augustus-gene- | | subfamily IG, 5′-nucleotidase |
| | | | CAT[G/C]TTCGTACCGTGCATGGC | | | | 20.116 | | chr5: 19849543-19853564 FORWARD |
| | | | TCTCTGCACATACCCAAATCTATC | | | | | | LENGTH = 4022 |
| | | | AGCCTTGAC (SEQ ID NO: 156) | | | | | | |
| 20,827,570 | A | G | TACCGGAGAAGAATAAGCGGTG | TGT −> | C −> R | TS | maker-N6- | AT5G48960.1 | \| Symbols: \| HAD-superfamily hydrolase, |
| | | | AACCTCAGCTTCTTCGCCATATCT | CGT | | | augustus-gene- | | subfamily IG, 5′-nucleotidase \| |
| | | | CCAC[A/G]CATAAGCATTCTGTCG | | | | 20.116 | | chr5: 19849543-19853564 FORWARD |
| | | | AACACCTGATGGGCGCATAGATT | | | | | | LENGTH = 4022 |
| | | | CATTTTTCACT (SEQ ID NO: 157) | | | | | | |
| 20,827,573 | T | G | CGGAGAAGAATAAGCGGTGAAC | ATG −> | M −> L | TV | maker-N6- | AT5G48960.1 | \| Symbols: \| HAD-superfamily hydrolase, |
| | | | CTCAGCTTCTTCGCCATATCTCCA | CTG | | | augustus-gene- | | subfamily IG, 5′-nucleotidase \| |
| | | | CACA[T/G]AAGCATTCTGTCGAAC | | | | 20.116 | | chr5: 19849543-19853564 FORWARD |
| | | | ACCTGATGGGCGCATAGATTCAT | | | | | | LENGTH = 4022 |
| | | | TTTTCACTTTT (SEQ ID NO: 158) | | | | | | |
| 20,912,356 | A | C | AATGTTGTGGCAGGCGTTGTCTA | GAT −> | D −> A | TV | maker-N6- | AT5G48840.1 | \| Symbols: PANC, PTS, ATPTS \| homolog of |
| | | | TAAGTAGGTCACTGGCCATGGCT | GCT | | | fgenesh-gene- | | bacterial PANC \| chr5: 19803557-19805077 |
| | | | AAAG[A/C]TTCTGCACAACAAGG | | | | 20.387 | | REVERSE LENGTH = 1521 |
| | | | GCAAACCAGTTGTAAAGAGCTTA | | | | | | |
| | | | AGGATATGATAA (SEQ ID NO: 159) | | | | | | |
| 20,912,364 | C | G | GGCAGGCGTTGTCTATAAGTAGG | CAA −> | Q −> E | TV | maker-N6- | AT5G48840.1 | \| Symbols: PANC, PTS, ATPTS \| homolog of |
| | | | TCACTGGCCATGGCTAAAGATTCT | GAA | | | fgenesh-gene- | | bacterial PANC \| chr5: 19803557-19805077 |
| | | | GCA[C/G]AACAAGGGCAAACCAG | | | | 20.387 | | REVERSE LENGTH = 1521 |
| | | | TTGTAAAGAGCTTAAGGATATGA | | | | | | |
| | | | TAATTTCAGAG (SEQ ID NO: 160) | | | | | | |
| 20,912,385 | A | G | GGTCACTGGCCATGGCTAAAGAT | AAA −> | K −> E | TS | maker-N6- | AT5G48840.1 | \| Symbols: PANC, PTS, ATPTS \| homolog of |
| | | | TCTGCACAACAAGGGCAAACCAG | GAA | | | fgenesh-gene- | | bacterial PANC \| chr5: 19803557-19805077 |
| | | | TTGT[A/G]AAGAGCTTAAGGATA | | | | 20.387 | | REVERSE LENGTH = 1521 |
| | | | TGATAATTTCAGAGATTGTTGGA | | | | | | |
| | | | GCTGCAGGAAGA (SEQ ID NO: 161) | | | | | | |
| 20,912,629 | C | G | GTGTGTACAACATGCAGATTGTT | CAG −> | Q −> E | TV | maker-N6- | AT5G48840.1 | \| Symbols: PANC, PTS, ATPTS \| homolog of |
| | | | GATCAAGAAACTCTTGAAGGAGT | GAG | | | fgenesh-gene- | | bacterial PANC \| chr5: 19803557-19805077 |
| | | | AGAA[C/G]AGATAGAGAGTGGA | | | | 20.387 | | REVERSE LENGTH = 1521 |
| | | | GTAGTGATTTGTGTTGCTGCCTG | | | | | | |
| | | | GTTTGGAACGGTT (SEQ ID NO: 162) | | | | | | |
| 20,912,710 | GCA | ACT | CTGCCTGGTTTGGAACGGTTAGG | GCA −> | A −> T | Substi- | maker-N6- | AT5G48840.1 | \| Symbols: PANC, PTS, ATPTS \| homolog of |
| | | | CTCATAGACAACATTGAGATCAAT | ACT | | tution | fgenesh-gene- | | bacterial PANC \| chr5: 19803557-19805077 |
| | | | GTC[GCA/ACT]GTCTAATTCCCAC | | | | 20.387 | | REVERSE LENGTH = 1521 |
| | | | TTTCCGCATCCATCTCTGGTTCGT | | | | | | |
| | | | TGACAGGAGCTTC (SEQ ID NO: 163) | | | | | | |
| 20,926,866 | C | G | AAAATCCGAGCTCTTGTTCTGGTT | GCT −> | A −> P | TV | augustus_maske | AT5G48810.1 | \| Symbols: ATB5-B, B5 #3, ATCB5-D, CB5-D |
| | | | AGACTGGGTCTGGTTTGAAGGAG | CCT | | | d-N6-abinit- | | \| cytochrome B5 isoform D \| |
| | | | GAG[C/G]AACAAACTTGGTTTTG | | | | gene-20.316 | | chr5: 19789067-19790334 REVERSE |
| | | | GCTGGGACAGTGGCGGAGTCAA | | | | | | LENGTH = 1268 |
| | | | TATCACCGACGT (SEQ ID NO: 164 | | | | | | |
| 20,926,884 | C | T | CTGGTTAGACTGGGTCTGGTTTG | GCC −> | A −> T | TS | augustus_maske | AT5G48810.1 | \| Symbols: ATB5-B, B5 #3, ATCB5-D, CB5-D |
| | | | AAGGAGGAGCAACAAACTTGGTT | ACC | | | d-N6-abinit- | | \| cytochrome B5 isoform D \| |
| | | | TTGG[C/T]TGGGACAGTGGCGGA | | | | gene-20.316 | | chr5: 19789067-19790334 REVERSE |
| | | | GTCAATATCACCGACGTAGTACTC | | | | | | LENGTH = 1268 |
| | | | ATCAAGCATGG (SEQ ID NO: 165) | | | | | | |
| 20,927,474 | T | G | CGTCGATGACGATCCAACAGTCC | GAG −> | E −> A | TV | augustus_maske | AT5G48810.1 | \| Symbols: ATB5-B, B5 #3, ATCB5-D, CB5-D |
| | | | TGGTTGCTAGTGTGCTGAGAAAC | GCG | | | d-N6-abinit- | | \| cytochrome B5 isoform D \| |
| | | | CTCC[T/G]CCAAGGTGAACACTTT | | | | gene-20.316 | | chr5: 19789067-19790334 REVERSE |
| | | | GCCGTCTCCGCCCATCTCCCGATG | | | | | | LENGTH = 1268 |
| | | | ATCCTCAATT (SEQ ID NO: 166) | | | | | | |
| 21,136,893 | G | C | AGCGTTCGGATTCTTTACGAACAT | CGA −> | R −> P | TV | maker-N6- | AT5G48100.1 | \| Symbols: TT10, LAC15, ATLAC15 \| |
| | | | CAAAAGCTTATACTCCGGACAAG | CCA | | | augustus-gene- | | Laccase/Diphenol oxidase family protein \| |
| | | | TTC[G/C]AGTCAAAATCTCACGTA | | | | 21.546 | | chr5: 19489327-19492744 REVERSE |
| | | | GAATAATCTCAACGGTTTCACTAA | | | | | | LENGTH = 3418 |
| | | | ATCTTCTAG (SEQ ID NO: 167) | | | | | | |
| 21,137,125 | T | G | GGAACGCGGTTTCCTGCGTTTCC | GAT −> | D −> E | TV | maker-N6- | AT5G48100.1 | \| Symbols: TT10, LAC15, ATLAC15 \| |
| | | | GCCTCTGGTTTTCAATTTTACCGC | GAG | | | augustus-gene- | | Laccase/Diphenol oxidase family protein \| |
| | | | GGA[T/G]GATCAACCGTTGATTTT | | | | 21.546 | | chr5: 19489327-19492744 REVERSE |
| | | | GCAGACTTCGAGACTTGCTACGG | | | | | | LENGTH = 3418 |
| | | | AAGTAAAGAT (SEQ ID NO: 168) | | | | | | |
| 21,137,150 | T | C | CTCTGGTTTTCAATTTTACCGCGG | TCG −> | S −> P | TS | maker-N6- | AT5G48100.1 | \| Symbols: TT10, LAC15, ATLAC15 \| |
| | | | ATGATCAACCGTTGATTTTGCAGA | CCG | | | augustus-gene- | | Laccase/Diphenol oxidase family protein \| |
| | | | CT[T/C]CGAGACTTGCTACGGAA | | | | 21.546 | | chr5: 19489327-19492744 REVERSE |
| | | | GTAAAGATGATTAAGTACGGAGA | | | | | | LENGTH = 3418 |
| | | | AGCGGTTGAA (SEQ ID NO: 169) | | | | | | |
| 21,137,202 | C | T | GAGACTTGCTACGGAAGTAAAGA | ACG −> | T −> M | TS | maker-N6- | AT5G48100.1 | \| Symbols: TT10, LAC15, ATLAC15 \| |
| | | | TGATTAAGTACGGAGAAGCGGTT | ATG | | | augustus-gene- | | Laccase/Diphenol oxidase family protein \| |
| | | | GAAA[C/T]GGTTTTTCAAGGGAC | | | | 21.546 | | chr5: 19489327-19492744 REVERSE |
| | | | GAGTTTAGGTGGTGGTGGAATCG | | | | | | LENGTH = 3418 |
| | | | ACCACCCCATGC (SEQ ID NO: 170) | | | | | | |
| 21,194,862 | G | A | ACTATGATCTCAGAAGTTTTGGAC | ACC −> | T −> 1 | TS | maker-N6- | AT5G47860.1 | \| Symbols: \| Protein of unknown function |
| | | | CCATATTTGAAGCCATCCAAGAT | ATC | | | augustus-gene- | | (DUF1350) \| chr5: 19381519-19384422 |
| | | | GTG[G/A]TTGCATCGACGAGCTG | | | | 21.614 | | FORWARD LENGTH = 2904 |
| | | | GGCAAGATCTTTGGACGTGTATG | | | | | | |
| | | | CACGAAGAATC (SEQ ID NO: 171) | | | | | | |
| 21,194,879 | GGC | AGT | TTTGGACCCATATTTGAAGCCATC | GCC −> | A −> T | Substi- | maker-N6- | AT5G47860.1 | \| Symbols: \| Protein of unknown function |
| | | | CAAGATGTGGTTGCATCGACGAG | ACT | | tution | augustus-gene- | | (DUF1350) \| chr5: 19381519-19384422 |
| | | | CTG[GGC/AGT]AAGATCTTTGGA | | | | 21.614 | | FORWARD LENGTH = 2904 |
| | | | CGTGTATGCACGAAGAATCTTCG | | | | | | |
| | | | AGTCCATTCCAAGCG (SEQ ID NO: 172) | | | | | | |
| 21,194,895 | G | A | AAGCCATCCAAGATGTGGTTGCA | ACG −> | T −> M | TS | maker-N6- | AT5G47860.1 | \| Symbols: \| Protein of unknown function |
| | | | TCGACGAGCTGGGCAAGATCTTT | ATG | | | augustus-gene- | | (DUF1350) \| chr5: 19381519-19384422 |
| | | | GGAC[G/A]TGTATGCACGAAGAA | | | | 21.614 | | FORWARD LENGTH = 2904 |
| | | | TCTTCGAGTCCATTCCAAGCGTAC | | | | | | |
| | | | CTCCAACCTCT (SEQ ID NO: 173) | | | | | | |
| 21,194,899 | A | C | CATCCAAGATGTGGTTGCATCGA | TAC −> | Y −> D | TV | maker-N6- | AT5G47860.1 | \| Symbols: \| Protein of unknown function |
| | | | CGAGCTGGGCAAGATCTTTGGAC | GAC | | | augustus-gene- | | (DUF1350) \| chr5: 19381519-19384422 |
| | | | GTGT[A/C]TGCACGAAGAATCTTC | | | | 21.614 | | FORWARD LENGTH = 2904 |
| | | | GAGTCCATTCCAAGCGTACCTCCA | | | | | | |
| | | | ACCTCTTTAG (SEQ ID NO: 174) | | | | | | |
| 21,196,738 | G | C | ACTCCATCCAATACAGCTAAGCTA | CTC −> | L −> V | TV | maker-N6- | AT5G47860.1 | \| Symbols: \| Protein of unknown function |
| | | | CTGCGAAAGAAAATCAATCACCT | GTC | | | augustus-gene- | | (DUF1350) \| chr5: 19381519-19384422 |
| | | | CGA[G/C]TTCTGACCAGTCGGCG | | | | 21.614 | | FORWARD LENGTH = 2904 |
| | | | GATCCAGCGGAGCTACTTTGAGA | | | | | | |
| | | | TTGCTTGACAG (SEQ ID NO: 175) | | | | | | |
| 21,197,016 | A | G | GACTCTTCGTGAGACGCGCTCTTT | TCT −> | S −> P | TS | maker-N6- | AT5G47860.1 | \| Symbols: \| Protein of unknown function |
| | | | GTCGAATCTGATCACCGAGAATG | CCT | | | augustus-gene- | | (DUF1350) \| chr5: 19381519-19384422 |
| | | | GAG[A/G]CCGGCGAGTGGAAGA | | | | 21.614 | | FORWARD LENGTH = 2904 |
| | | | GAAGTTAGAGAGTCTCCGGCGG | | | | | | |
| | | | GGAGAGAAAGACG (SEQ ID NO: 176) | | | | | | |

It can be hypothesized that some of these genes which contain SNPs in genes which have or are predicted to have a function in lipid biosynthesis or a related pathway in the parent which increases PUFA could be contributing to the increase in PUFA by some functional difference in those genes. For example, a desaturase enzyme may prefer different substrates depending on minor changes in its substrate binding pocket, which could impact the amount of PUFA produced.

### Example 2: Crosses/Hybrids

As demonstrated herein, adding certain genetic elements (QTL) can increase the amount of very long chain PUFAs. However, in a hybrid production system, the combination of male and female parents both containing very long chain PUFAs can yield a mid-parent amount of EPA+DPA+DHA, and in some cases even lower EPA+DPA+DHA than either parent.

Surprisingly, when hybrids were produced using a female heterozygous for the three QTL as previously described (and a male that is homozygous, heterozygous, or lacking all three QTL described herein, but all plants contain one or more tDNAs as described herein), the F1 hybrid seed demonstrated heterosis, that is it yielded higher EPA+DPA+DHA than either of the parents. In this case, the female alone produced 18.7% EPA+DPA+DHA, the male produced 13.6-14.8% EPA+DPA+DHA (in this case the male did not have the three QTL as described herein), and the F1 hybrid made from this cross produced 20.2-24.8% EPA+DPA+DHA. The same effect was observed when the female was heterozygous for the three QTL and the male was homozygous for the three QTL. In this case, the female alone produced 18.7% EPA+DPA+DHA, the male produced 13.8-14.5% EPA+DPA+DHA, and the F1 hybrid made from this cross produced 18.8-21.8% EPA+DPA+DHA.

### BIBLIOGRAPHY

Clarke et al. A high-density SNP genotyping array for Brassica napus and its ancestral diploid species based on optimised selection of single-locus markers in the allotetraploid genome. Theor Appl Genet (2016) 129:1887-1899.
Endelman, J. B. Ridge regression and other kernels for genomic selection with R package rrBLUP. Plant Gen. (2011) 4(3):250-255.
Lipka, A., Tian, Q. Wang, J. Peiffer, M. Li, P. Bradbury, M. Gore, E. Buckler, and Z. Zhang. GAPIT: genome association and prediction integrated tool. Bioinformatics (2012) 28: 2397-2399
R Development Core Team. R: a language and environment for statistical computing. R Foundation for Statistical Computing. Vienna, Austria. (2015) http://www.R-project.org (accessed 31 Jul. 2016).

All publications, patents, and patent applications mentioned in this specification are herein incorporated by reference to the same extent as if each individual publication, patent, or patent application was specifically and individually indicated to be incorporated by reference. In the event that the definition of a term incorporated by reference conflicts with a term defined herein, this specification shall control.

1. (canceled)

2. A Brassica plant or a part thereof comprising:
(i) one or more T-DNAs heritably integrated into its genome, the T-DNAs comprising one or more expression cassettes having nucleotide sequences encoding one or more d12DES, one or more d6Elo, one or more d6Des, one or more d5Des, one or more d5Elo, one or more d4Des, one or more o3Des; or a combination thereof; and
(ii) all or part of at least one genomic sequence of a B. napus parent genome that confers a higher polyunsaturated fatty acid content, wherein said genome sequence is selected from the group consisting of:
a) the genomic sequence on chromosome N1 between nucleotide positions 8,879,780 and 11,922,690;
b) the genomic sequence on chromosome N1 between nucleotide positions 22,823,086 and 24,045,492; and
c) the genomic sequence on chromosome N6 between nucleotide positions 19,156,645 and 20,846,412;
wherein seeds of said Brassica plant have a greater amount of one or more polyunsaturated fatty acids selected from the group consisting of EPA, DPA, and DHA than seeds of a control Brassica plant lacking (i) a (ii).

3. The Brassica plant or part thereof of claim 2, wherein said genomic sequence on chromosome N1 between nucleotide positions 8,879,780 and 11,922,690 comprises a single nucleotide polymorphism (SNP) at a position selected from the group 8,952,616, 9,040,901, 9,046,609, 9,048,617, 9,136,686, 9,143,608, 9,248,592, 9,347,120, 9,352,326, 9,454,361, 9,549,523, 9,641,936, 9,652,028, 9,794,198, 9,847,417, 9,921,975, 9,952,792, 10,052,015, 10,402,684, 10,425,211, 10,558,464, 10,613,015, 10,659,284, 10,706,805, 10,748,492, 10,852,010, 11,007,740, 11,047,958, 11,150,929, 11,269,217, 11,343,118, 11,455,979, 11,565,970, 11,659,776, 11,726,807, and 11,850,103.

4.-8. (canceled)

9. The Brassica plant or part thereof of claim 2, wherein said genomic sequence on chromosome N1 between nucleotide positions 8,879,780 and 11,922,690 comprises a SNP at a position selected from the group consisting of 9,136,686, 9,641,936, 10,613,015, 9,040,901, 9,048,617, 9,352,326, 9,921,975, and 10,706,805.

10.-13. (canceled)

14. The Brassica plant or part thereof of claim 2, wherein said genomic sequence on chromosome N1 between nucleotide positions 22,823,086 and 24,045,492 comprises a SNP at a position selected from the group 22,823,086, 22,880,595, 22,902,670, 22,949,738, 23,011,207, 23,044,228, 23,099,592, 23,176,771, 23,201,595, 23,257,618, 23,302,268, 23,367,822, 23,380,089, 23,457,696, 23,520,607, 23,552,773, 23,598,941, 23,670,623, 23,682,848, 23,745,365, 23,792,572, 23,855,829, 23,910,029, 23,947,522, and 24,021,883.

15.-18. (canceled)

19. The Brassica plant or part thereof of claim 2, wherein said genomic sequence on chromosome N1 between nucleotide positions 22,823,086 and 24,045,492 comprises a SNP at a position selected from the group 23,089,542, 23,089,635, 23,090,743, 23,090,785, 23,091,367, 23,092,042, 23,150,402, 23,150,595, 23,155,220, 23,155,766, 23,314,197, 23,318,357, 23,343,089, 23,679,276, 23,679,287, 23,679,396, 23,886,929, 23,925,895, 23,963,309, 24,029,270, 24,029,279, and 24,029,294.

20.-23. (canceled)

24. The Brassica plant or part thereof of claim 2, wherein said genomic sequence on chromosome N6 between nucleotide positions 19,156,645 and 20,846,412 comprises a SNP at a position selected from the group 19,156,645, 19,199,109, 19,325,186, 19,402,086, 19,513,420, 19,583,431, 19,601,021, 19,706,563, 19,800,643, 19,906,666, 20,000,119, 20,095,002, 20,205,211, 20,300,571, 20,406,148, 20,407,023, 20,505,840, 20,601,198, 20,631,917, and 20,702,631.

25.-29. (canceled)

30. The Brassica plant or part thereof of claim 2, wherein said genomic sequence on chromosome N6 between nucleotide positions 19,156,645 and 20,846,412 comprises a SNP at a position selected from the group 19,336,744, 19,336,819, 19,337,615, 19,350,156, 19,353,584, 19,353,648, 19,353,749, 19,476,836, 19,783,834, 19,784,007, 19,784,367, 19,784,633, 19,784,672, 19,784,688, 19,784,733, 19,800,525, 20,191,826, 20,300,548, 20,375,643, 20,766,637, 20,769,461, 20,770,769, 20,823,998, 20,825,959, 20,826,301, 20,827,570, and 20,827,573.

31.-39. (canceled)

40. The Brassica plant or part thereof of claim 2, wherein said expression cassettes comprise nucleotide sequences encoding:
a) one or more d12DES a one or more d6Elo;
b) one or more d6Elo one or more d6Des;
c) one or more d6Des a one or more d5Des;
d) one or more d5Des a one or more d5Elo;
e) one or more d5Elo a one or more d4Des;
f) one or more d4Des a one or more o3Des;
g) one or more d12DES a one or more d6Des;
h) one or more d12DES a one or more d5Des;
i) one or more d12DES a one or more d5Elo;
j) one or more d12DES & one or more d4Des;
k) one or more d12DES, Wd one or more o3Des;
l) one or more d6Elo Wd one or more d5Des;
m) one or more d6Elo Wd one or more d5Elo;
n) one or more d6Elo & one or more d4Des; and/or
o) one or more d6Elo & one or more o3Des.

41. The Brassica plant or the part thereof of claim 2, wherein said one or more expression cassettes comprise at least two nucleotide sequences encoding a d6Des, at least two nucleotide sequences encoding a d6Elo, at least two nucleotide sequences encoding an o3Des and combinations thereof.

42. The Brassica plant or part thereof of claim 2, wherein the one or more expression cassettes comprise at least one nucleotide sequences encoding CoA-dependent d4Des a at least one nucleotide sequence encoding phospholipid dependent d4Des.

43. The Brassica plant or part thereof of claim 2, wherein the one or more expression cassettes comprise nucleotide sequences encoding at least one, or at least two, d12Des.

44. The Brassica plant or part thereof of claim 2, wherein said seeds further comprises a greater amount of one or more polyunsaturated fatty acids selected from the group consisting of eicosadienoic acid, dihomo-gamma linolenic acid, and arachidonic acid.

45. The Brassica plant or part thereof of claim 2, wherein said seeds have an EPA content of from about 11.5% to about 15%.

46. The Brassica plant or part thereof of claim 2, wherein said seeds have a DHA content of from about 0.9% to about 1.5%

47. The Brassica plant or part thereof of claim 2, wherein said seeds have a DPA content of from about 3.5% to about 5%.

48. (canceled)

49. The Brassica plant or part thereof of claim 2, wherein said plant is selected from the group consisting of Brassica napus, Brassica oleracea, Brassica juncea, Brassica nigra, Brassica rapa, and Brassica carinata.

50. The Brassica plant or part thereof of claim 49, wherein said plant is selected from the group consisting of Brassica napus, Brassica rapa, and Brassica juncea.

51. The Brassica plant or part thereof of claim 2, wherein said plant is tolerant of an herbicide.

52. The Brassica plant or part thereof of claim 51, wherein said herbicide is selected from the group consisting of imidazolinone, dicamba, cyclohexanedione, sulfonylurea, glyphosate, glufosinate, phenoxy propionic acid, L-phosphinothricin, triazine, and benzonitrile.

53. The Brassica plant or part thereof of claim 2, wherein said plant further comprises a gene encoding a Bacillus thuringiensis endotoxin, and wherein said endotoxin is produced in said Brassica plant or said part thereof.

54. A method of producing a Brassica plant or a part thereof, said method comprising crossing a first Brassica parent plant producing one or more of DPA, DHA and/or EPA in its seeds with a second Brassica parent plant to produce progeny plants, wherein one or both of said first and said second Brassica parent plants comprises all or part of at least one genomic sequence of a B. napus parent genome that confers a higher polyunsaturated fatty acid content,
wherein said genome sequence is selected from the group consisting of:
a) the genomic sequence on chromosome N1 between nucleotide positions 8,879,780 and 11,922,690;
b) the genomic sequence on chromosome N1 between nucleotide positions 22,823,086 and 24,045,492; and
c) the genomic sequence on chromosome N6 between nucleotide positions 19,156,645 and 20,846,412; and
wherein seeds of said Brassica plant have a greater amount of one or more polyunsaturated fatty acids selected from the group consisting of EPA, DPA, and DHA than seeds of said first parental Brassica plant and/or the second parental Brassica plant.

55. The method of claim 54, wherein the first Brassica plant comprises one or more T-DNAs heritably integrated into its genome, the T-DNAs comprising one or more expression cassettes having nucleotide sequences encoding one or more d12DES, one or more d6Elo, one or more d6Des, one or more d5Des, one or more d5Elo, one or more d4Des, and/or one or more o3Des, wherein the one or more genes are heritably integrated into the plant genome.

56.-59. (canceled)

60. The method of claim 54, wherein progeny are selected that produce a greater amount of DPA, DHA and/or EPA in their seed than said first Brassica parent plant and/or said second Brassica parent plant, and which comprise all or part of said genomic sequences.

61.-67. (canceled)


SEQUENCE LISTING (177 sequences)

SEQ ID NO: 1 - DNA, 101 bp, Brassica napus
  variation 51: note = n = T or C
  source 1..101: mol_type = genomic DNA
atttgagagtgggaaaagactttgtaggtagtagatacagagtccaagaanctatccagggtcttggtgtagctgtcaacattcatgacgcagatgacatt

SEQ ID NO: 2 - DNA, 101 bp, Brassica napus
  variation 51: note = n = T or C
  source 1..101: mol_type = genomic DNA
ctgatacatttaatagattaccgtaaaacaagtttgctgtacaccagcttnggaggtgctgagagcacttctgcttctgcagaaacgggaagataaacaac

SEQ ID NO: 3 - DNA, 101 bp, Brassica napus
  variation 51: note = n = G or A
  source 1..101: mol_type = genomic DNA
tttgtcgtcaacgtcgccttcttccacatcccccatataaaccttaaacancaacaagagatttttaatggaaactgatgaagactgatctaatagtcata

SEQ ID NO: 4 - DNA, 101 bp, Brassica napus
  variation 51: note = n = G or A
  source 1..101: mol_type = genomic DNA
tcattgctaatgcttttgttttgaacaccatttgcctcatctaagctcggntgaacttcagtgaccgtggtcaagaaatctaaaccaaggacggcgcatag

SEQ ID NO: 5 - DNA, 101 bp, Brassica napus
  variation 51: note = n = G or A
  source 1..101: mol_type = genomic DNA
agttggacctgagcttagtttccttaaagacgtgggacgggctgagtactncagggttaccgatgaagaagccttagaaggtgctctactacttacttggc

SEQ ID NO: 6 - DNA, 101 bp, Brassica napus
  variation 51: note = n = C or T
  source 1..101: mol_type = genomic DNA
acatatcttattggttatacatgatcctcgtttctgtcatcttcatactcngaaacaaaaaaattaaaaatcaccatattaattgcaaagttgtatcaatt

SEQ ID NO: 7 - DNA, 101 bp, Brassica napus
  variation 51: note = n = G or A
  source 1..101: mol_type = genomic DNA
ccgttgagaatcgtcctgagcgaattcatccgaggaagatcaaaagaccgntctttcatctccgggaacgaatcaagaacgtcgtcgagctgagacgaaga

SEQ ID NO: 8 - DNA, 101 bp, Brassica napus
  variation 51: note = n = C or T
  source 1..101: mol_type = genomic DNA
ctcccatctgtcactgcagtacttgaacagctcccaaagtgctaaacttgntaacaaccccaccaatgcaagtggcatctgatacctacaaagaaacacca

SEQ ID NO: 9 - DNA, 101 bp, Brassica napus
  variation 51: note = n = C or T
  source 1..101: mol_type = genomic DNA
aaccatatctactcgtaggttggtcttgatcgattctagttgagaagtatngatcggtgccagttcacaagtgtcgatcgatgatgtgcgtctttttttgc

SEQ ID NO: 10 - DNA, 101 bp, Brassica napus
  variation 51: note = n = G or T
  source 1..101: mol_type = genomic DNA
ttcgcagtggttccggtgctttttcgacattcgttgaaagaaggggaggangatagagtgacgagcttggatcacatattcagcgttgagccgatgaagat

SEQ ID NO: 11 - DNA, 101 bp, Brassica napus
  variation 51: note = n = C or T
  source 1..101: mol_type = genomic DNA
accaaactctggatacgaagcttctggtccaagatccagagaagatcaagntccaccagtgccacaaccatgacttgaaaacagtcaacaaataagcgttt

SEQ ID NO: 12 - DNA, 101 bp, Brassica napus
  variation 51: note = n = G or A
  source 1..101: mol_type = genomic DNA
atttcgatagagtcgctggagagagcgactccaaagccattaagaaaccgnttgaatcaaagtccatcgtgaaggataagaagaagaagactgagtcaagc

SEQ ID NO: 13 - DNA, 101 bp, Brassica napus
  variation 51: note = n = G or A
  source 1..101: mol_type = genomic DNA
tgggtaatctcagccactactcgggctctgggttgagcgggcttggcgganccgggtcgaacaatcctggttctcccggtgatggccatgaccacggcgtc

SEQ ID NO: 14 - DNA, 101 bp, Brassica napus
  variation 51: note = n = A or C
  source 1..101: mol_type = genomic DNA
atagtcaacagccgtctcaagaacatcatcctaaggtgtcccaaccaggtngcacccgttaagtttctacagaagaagttcaagactcttgatctccaagg

SEQ ID NO: 15 - DNA, 101 bp, Brassica napus
  variation 51: note = n = A or C
  source 1..101: mol_type = genomic DNA
tgcataagtactactcgaacccatgtcataaaatataacatgtctcgaccnattagagaaatccttatcaatcccatactgcaaagccgcaccggaatgct

SEQ ID NO: 16 - DNA, 101 bp, Brassica napus
  variation 51: note = n = A or T
  source 1..101: mol_type = genomic DNA
gagctcgagttcctcaaccacgaactaaacagtagcggggaagacaaagcngaagaacgtaaaggtcaagcagaagctgaagaagacgaagatgagaagcc

SEQ ID NO: 17 - DNA, 101 bp, Brassica napus
  variation 51: note = n = T or C
  source 1..101: mol_type = genomic DNA
aagtttggtgttgacctgttaacatgttctagctttcaggtagagcatcanagaaagaaagagcagcctgacaagacactacaaggtctttgcatcactgg

SEQ ID NO: 18 - DNA, 101 bp, Brassica napus
  variation 51: note = n = A or G
  source 1..101: mol_type = genomic DNA
tcacaacctaagtcatattcatattaccaattgcagttactgtgaatgagnttgaagctctttacgaaatgttcaagagcatcagcaaagacggccttatc

SEQ ID NO: 19 - DNA, 101 bp, Brassica napus
  variation 51: note = n = G or A
  source 1..101: mol_type = genomic DNA
gactatgctgctttcgagagtaagagcttcactgtacaatgctgagtcagntggtgtctcagtatgatcttctgtgaagccaagtctgtgagagacatgag

SEQ ID NO: 20 - DNA, 101 bp, Brassica napus
  variation 51: note = n = G or A
  source 1..101: mol_type = genomic DNA
tcaccaaactcacatgtctgtttcttgacgtctcttgcacacactttgtanaagattgcaataatattaagctttccttgttccacacgtttcttcatcat

SEQ ID NO: 21 - DNA, 101 bp, Brassica napus
  variation 51: note = n = G or A
  source 1..101: mol_type = genomic DNA
ttccatcaggaggttctgatgattttatggtgcaaaatgaggggagtatangttctaagcaactcatcgaggaggatgggggtggtagcattcaagaatct

SEQ ID NO: 22 - DNA, 101 bp, Brassica napus
  variation 51: note = n = G or C
  source 1..101: mol_type = genomic DNA
ttatctctagcaagttacaatgagatattggatggaagtgttgatactaantgtcttattggtatataattttgaactgcttctcttagattatttttatc

SEQ ID NO: 23 - DNA, 101 bp, Brassica napus
  variation 51: note = n = G or A
  source 1..101: mol_type = genomic DNA
ctctcctcgcttggtgaaaccggagagaccattggtttgcctcattgtcgncatctttcctcttctaccggtaagaatcttgtgaggataagactgatgac

SEQ ID NO: 24 - DNA, 101 bp, Brassica napus
  variation 51: note = n = G or A
  source 1..101: mol_type = genomic DNA
cctgtctctaacatctgctgcagagttacccgcttccaccatatctccacngagcctgtacgcgattaggcgaagaggactatctcttggttctttcttga

SEQ ID NO: 25 - DNA, 101 bp, Brassica napus
  variation 51: note = n = G or C
  source 1..101: mol_type = genomic DNA
tccgaaaccttgaccaaattctaccccctcgcgggaagaatcaacggagtnactgtcgactgtaacgacgaaggagctgttttcgtcgacgctcgtgtcga

SEQ ID NO: 26 - DNA, 101 bp, Brassica napus
  variation 51: note = n = A or T
  source 1..101: mol_type = genomic DNA
ggcttggtctggtaggcataatgaaagccgcttgacatgtgtgattaaacnaacaaagttctgtatgtcaaggtatgtcgaatcgacttccatctcctcca

SEQ ID NO: 27 - DNA, 101 bp, Brassica napus
  variation 51: note = n = C or T
  source 1..101: mol_type = genomic DNA
aagagcgatgggcttggtgacttttcttcctttccggagacgagtgattcngagtctagcgataagcccctcatggcacactatggcaatgtagaagagcc

SEQ ID NO: 28 - DNA, 101 bp, Brassica napus
  variation 51: note = n = C or G
  source 1..101: mol_type = genomic DNA
gatctgtcgagcttagtcgctatccacggataatactcataatgactttcntttcctaattgagatgaagctctatgcacccgtacggccttaagtacacc

SEQ ID NO: 29 - DNA, 101 bp, Brassica napus
  variation 51: note = n = C or T
  source 1..101: mol_type = genomic DNA
gtgtatatcgttaaccccacgaccattgtagctgtgagaaccgcagcctcnagaacaatcttccctgttttttttattcacaagagttagattgtaacatc

SEQ ID NO: 30 - DNA, 101 bp, Brassica napus
  variation 51: note = n = A or G
  source 1..101: mol_type = genomic DNA
aagcactcgacttataaccatgtttgatatgtttcgacgatctaccagtcngcttctgtgaagtctgctcagcaacgtggtcagccctttcatgaccactc

SEQ ID NO: 31 - DNA, 101 bp, Brassica napus
  variation 51: note = n = C or T
  source 1..101: mol_type = genomic DNA
caccatcaccagctatgggctttccatctccaccatcaccagaaaaagccncagggaccattctgctcggtaaatacgaactcggtcgccgattaggcagt

SEQ ID NO: 32 - DNA, 101 bp, Brassica napus
  variation 51: note = n = A or C
  source 1..101: mol_type = genomic DNA
tagttgagaaacgttttgtgagtcctcctctttccaacgacccaactctgnagtctacatggactcaccgcttatgggttgcagctggttgcaccaccttg

SEQ ID NO: 33 - DNA, 101 bp, Brassica napus
  variation 51: note = n = G or A
  source 1..101: mol_type = genomic DNA
tattgataagtatggattcgagtaccaattcaatggtaaaaaagatgtcangtcttacctgctttagattatttaagaaacttcttgttagatatgcctga

SEQ ID NO: 34 - DNA, 101 bp, Brassica napus
  variation 51: note = n = C or G
  source 1..101: mol_type = genomic DNA
tttaaacaggttgcttgaacggtggtgggcagattaagccaaaaacggganagactccgaaagaactgatcaactcacttgaagctacttatatgaatgat

SEQ ID NO: 35 - DNA, 101 bp, Brassica napus
  variation 51: note = n = A or G
  source 1..101: mol_type = genomic DNA
aggaggatgagaattcgtgttaccggaagagtagcatagctaagggccaanttgattacagcggctgcgataagcgatacaatgatattgaaaccaagcat

SEQ ID NO: 36 - DNA, 101 bp, Brassica napus
  variation 51: note = n = C or A
  source 1..101: mol_type = genomic DNA
tatattaatccaggacattggcattgtaagaaaaatcattttcaaaagggnaatggagaaagatctgaatcagaactctccacgaatcagaaagctaaggg

SEQ ID NO: 37 - DNA, 101 bp, Brassica napus
  variation 51: note = n = A or G
  source 1..101: mol_type = genomic DNA
agcaaatacatattatatttatatattaaaatacctgatcttgtaagtganaaaggtccgttttccaccaaaggagaccacataagaaagacacgacaaaa

SEQ ID NO: 38 - DNA, 102 bp, Brassica napus
  variation 51: note = n = T or G
  variation 52: note = n = G or A
  source 1..102: mol_type = genomic DNA
ggatatgattgagacagggattaatgatcaatgctcaagcgttgaagctannagcttcacacagctccttaggaacctctccagctcccctggctactgctt

SEQ ID NO: 39 - DNA, 101 bp, Brassica napus
  variation 51: note = n = G or T
  source 1..101: mol_type = genomic DNA
aaagcaatcgactcagcattttccctgacgcggacaaggttgtaacgaaantcagcctctttcttctcttgcataaagttgagattcactagtccctgaga

SEQ ID NO: 40 - DNA, 101 bp, Brassica napus
  variation 51: note = n = T or C
  source 1..101: mol_type = genomic DNA
caccatttctagatctatagaatcagaattcgaattggatcttcggtgaanctaatctccaatatcatctcctctgatctctaaagcttgaggaggataac

SEQ ID NO: 41 - DNA, 101 bp, Brassica napus
  variation 51: note = n = A or C
  source 1..101: mol_type = genomic DNA
atagagtaacgattatcatcaaccttagtgaaccctgagagatacttctcnacttcgtcccattcaccggcgagagctttctcttcaaagtactttatatt

SEQ ID NO: 42 - DNA, 101 bp, Brassica napus
  variation 51: note = n = G or A
  source 1..101: mol_type = genomic DNA
atgtttattcatttttcataagttttttttagttttcatttacattttatntatgattttatcaaactacataaataaaaaatctacaaactttaatcaaa

SEQ ID NO: 43 - DNA, 101 bp, Brassica napus
  variation 51: note = n = C or T
  source 1..101: mol_type = genomic DNA
cattatcctgaagcaatgagtttctaacctgtaaatatacttataacctgngtgtcctctcccccaaaactgcaactgagccatcatatgcttgagatcaa

SEQ ID NO: 44 - DNA, 101 bp, Brassica napus
  variation 51: note = n = C or T
  source 1..101: mol_type = genomic DNA
tcctcccacataggtcaaatcaataagaatggtgatggtgtgaagcaagtngaacctaagagggaaggcatgtttgtaaagatcaacatggacagtgttcc

SEQ ID NO: 45 - DNA, 101 bp, Brassica napus
  variation 51: note = n = C or G
  source 1..101: mol_type = genomic DNA
ctatattttataacaacaactttcgcaatatcatatttttatggattaatnatgtggtaataaaaattctagttctcccaaattaaggtacaaaattaatg

SEQ ID NO: 46 - DNA, 101 bp, Brassica napus
  variation 51: note = n = A or G
  source 1..101: mol_type = genomic DNA
cagcaagccagcctccagtcgcagatgagccgaatgatgaacctcctgctntggattttgcgttcgtccatgtaagtagtgatcttcactcgctagttgct

SEQ ID NO: 47 - DNA, 101 bp, Brassica napus
  variation 51: note = n = A or G
  source 1..101: mol_type = genomic DNA
ctcacagagctatacataaacaaaccatccacaacaagaagaaacgccatnaacgaaacaatacgaacgtgcgcgagcttagagacgttaggagtcgtctc

SEQ ID NO: 48 - DNA, 101 bp, Brassica napus
  variation 51: note = n = G or A
  source 1..101: mol_type = genomic DNA
tgataaggttactgcaaagaaagttgtcaggaggagaaagagcagtactanttcttgaggagacaaaggttataatcaccagttcttgctccagttattat

SEQ ID NO: 49 - DNA, 101 bp, Brassica napus
  variation 51: note = n = A or C
  source 1..101: mol_type = genomic DNA
agataatgtgactgattggcatgaactaatcacagaatctggacttcttgntaagagtcattttactgatagagctgcagaaaccggagaggcatcaatat

SEQ ID NO: 50 - DNA, 101 bp, Brassica napus
  variation 51: note = n = G or A
  source 1..101: mol_type = genomic DNA
ctggagcatgtactcctctacgcccgtgaagaacagaactcctaccgctancaaacacggtatccaatggactctcaccacgtggacaggattgcacatcc

SEQ ID NO: 51 - DNA, 101 bp, Brassica napus
  variation 51: note = n = T or C
  source 1..101: mol_type = genomic DNA
gactcagtgagagcccaagtccgaaaagccctagaagagtacggatgtttngaggccacgttcgatggagtttcagcggagctaaggaaggctattttcaa

SEQ ID NO: 52 - DNA, 101 bp, Brassica napus
  variation 51: note = n = A or C
  source 1..101: mol_type = genomic DNA
tgatcaggcttctctatgtcatcaatcttctctttaccaccatcattaagnttctctttgtaatcaggcattactttatgagcatgctttttttcaggatc

SEQ ID NO: 53 - DNA, 101 bp, Brassica napus
  variation 51: note = n = A or G
  source 1..101: mol_type = genomic DNA
aaagaagagcttagtcaacgcattgaagaaagatagtccggtgggccagantcttgttctttgtttgaatttggcattaccgggaaaggcatgtgagtgtg

SEQ ID NO: 54 - DNA, 101 bp, Brassica napus
  variation 51: note = n = C or T
  source 1..101: mol_type = genomic DNA
acagaactggtattaatagtagttgtaataacaatagtgtccagtgttgtncgatgggagggagtttacagagaactcagacgttacctagttacatagga

SEQ ID NO: 55 - DNA, 101 bp, Brassica napus
  variation 51: note = n = A or G
  source 1..101: mol_type = genomic DNA
cggggaggacgggaggctttttgacctcgaggtagggagtaggtgaggtgnggttttgggatggaggagagatgaggagaggggagaaatgtggtggtttg

SEQ ID NO: 56 - DNA, 101 bp, Brassica napus
  variation 51: note = n = C or T
  source 1..101: mol_type = genomic DNA
tgtggctttgggaaatcataaacgttgttgaatacttctttgatttgctcnggatccattatggtgatggttggtataggtccaaaccatgtatagtaagt

SEQ ID NO: 57 - DNA, 101 bp, Brassica napus
  variation 51: note = n = C or A
  source 1..101: mol_type = genomic DNA
tgacgagagcacagtcacggaacagataatcaacttgctttgaatagattnggacaacaccaagtaaaagatgaccagacaatctcaatgccaaggtgcct

SEQ ID NO: 58 - DNA, 101 bp, Brassica napus
  variation 51: note = n = T or A
  source 1..101: mol_type = genomic DNA
gggatgctaggaatgtttttcgagccacgatattcacttggaagaattcanaccgttaaacttacaatggtcctcactgttgtggatgatacatgtgatgc

SEQ ID NO: 59 - DNA, 101 bp, Brassica napus
  variation 51: note = n = C or A
  source 1..101: mol_type = genomic DNA
tcaatggcggcggatcggagcaaaggcgttgtgagagacgagttcgtgacntatgcttacattcttctctacatcgctctctctagcggtcaaatcttctt

SEQ ID NO: 60 - DNA, 101 bp, Brassica napus
  variation 51: note = n = G or A
  source 1..101: mol_type = genomic DNA
cggaacggtcgacttcatcttcggaaacgccgctgtcgttctccaaaactncgacatccacgctcgccgaccaaactccggccagaaaaacattgtcacgg

SEQ ID NO: 61 - DNA, 101 bp, Brassica napus
  variation 51: note = n = T or C
  source 1..101: mol_type = genomic DNA
atagagtccggtggtcaagctgagaagtctgctaagagaaagagaaagaancagtacagggggattaggcagcgaccttggggaaaatgggctgctgagat

SEQ ID NO: 62 - DNA, 101 bp, Brassica napus
  variation 51: note = n = C or T
  source 1..101: mol_type = genomic DNA
acgacgaccagatccgctctcactccgactcacccgatccgtcttcttctncgccgccgccgtcgggaaaagtcacggtaacggtggctttcccaggtccg

SEQ ID NO: 63 - DNA, 101 bp, Brassica napus
  variation 51: note = n = A or C
  source 1..101: mol_type = genomic DNA
gagaatcttgaaagcttctagaagcttctggaaaaaaagttctttccccanctccaactctctccccaatcccttgaaaatatctacaattaatcataatt

SEQ ID NO: 64 - DNA, 101 bp, Brassica napus
  variation 51: note = n = G or A
  source 1..101: mol_type = genomic DNA
cacagaacgctaacgcgccagcgtaagtgaacttatcgagcttcaaattgnaatctctagcttctaggaacagcttcatggagtgttcgtgaaacccaact

SEQ ID NO: 65 - DNA, 101 bp, Brassica napus
  variation 51: note = n = T or C
  source 1..101: mol_type = genomic DNA
ggcagagactaaagagacaagaaaccctagaagaaacagagcagaagctgntgccatagccacattcttcattactgttgaaagcaaaatatcttgcttta

SEQ ID NO: 66 - DNA, 101 bp, Brassica napus
  variation 51: note = n = A or T
  source 1..101: mol_type = genomic DNA
tagtagtgaatgcgagcatttgatctccgctgcttatgagtttctgttgtntaatggtttcattaactttggggtgtctccgttgtttaatggatatgttc

SEQ ID NO: 67 - DNA, 101 bp, Brassica napus
  variation 51: note = n = C or T
  source 1..101: mol_type = genomic DNA
cgatgaaatttgatcttcaaggaattggaaacgaaagagacttcattgatncatctataaagcaagtaagtatacttgaccaagtagagatcactcaagta

SEQ ID NO: 68 - DNA, 101 bp, Brassica napus
  variation 51: note = n = G or T
  source 1..101: mol_type = genomic DNA
ggaggagggaagattgattcaggtagccaagggagagataccatcaagaangggagtgaagagcgcatagattcggacttgagaagtagcgattttgtgtt

SEQ ID NO: 69 - DNA, 101 bp, Brassica napus
  variation 51: note = n = G or C
  source 1..101: mol_type = genomic DNA
gctacgttgcgccgtatctcatggacagttttgttcagtacctgaacggtnaaaggcaatacaaataccaaggatacgttttggtaacgattttcttcgtt

SEQ ID NO: 70 - DNA, 101 bp, Brassica napus
  variation 51: note = n = T or G
  source 1..101: mol_type = genomic DNA
ctcaatgatgaagaagatgagaatgatgatgctgttaaggagatgcaagancgtgtccacacaattaagtcgttcatattcctacacaagcctcagatccc

SEQ ID NO: 71 - DNA, 101 bp, Brassica napus
  variation 51: note = n = T or A
  source 1..101: mol_type = genomic DNA
atcatatggggaggcactggtagccgtgggaggttgaagctgaaagtaggngagtctccagagagctggactactggagttgatctgggaaggttacttac

SEQ ID NO: 72 - DNA, 101 bp, Brassica napus
  variation 51: note = n = A or C
  source 1..101: mol_type = genomic DNA
aaagctgcttcgatctgccaatcactggctttaagagcctgaagagcactnttctcactgctcccccagcacattattaaacaaatcacacacacaaaaaa

SEQ ID NO: 73 - DNA, 101 bp, Brassica napus
  variation 51: note = n = C or T
  source 1..101: mol_type = genomic DNA
gtaaataaagagtaacaatcattacattggcgacaaagattgttcgagacntagcatcttccaaagggcggctagtagataatgtccctacaaataccagt

SEQ ID NO: 74 - DNA, 101 bp, Brassica napus
  variation 51: note = n = C or G
  source 1..101: mol_type = genomic DNA
cttgaaaacgtagaagaaagcgttgcgttttatgagaagcagttctctgtnccaacagatcttgaagaggtcagatttggggacagagtgtttgttgaata

SEQ ID NO: 75 - DNA, 101 bp, Brassica napus
  variation 51: note = n = A or T
  source 1..101: mol_type = genomic DNA
gttgaatacgagagaagctggatgtgatttgaatctgtcgtaagggacggntagttcgttttcaagattggcaccgacatggaaggttttggactgtttgg

SEQ ID NO: 76 - DNA, 101 bp, Brassica napus
  variation 51: note = n = G or T
  source 1..101: mol_type = genomic DNA
aatggggaaaactagacctgttgtgactcttttcttttgaccacctgaaantcctcgtgtcatttcgtctccgaccatcgtatctttgcataagtcaagcc

SEQ ID NO: 77 - DNA, 101 bp, Brassica napus
  variation 51: note = n = A or T
  source 1..101: mol_type = genomic DNA
acctgaaattcctcgtgtcatttcgtctccgaccatcgtatctttgcatangtcaagccctaatatctaaaatagaaaatgatcaatagttgcccaaattt

SEQ ID NO: 78 - DNA, 101 bp, Brassica napus
  variation 51: note = n = T or C
  source 1..101: mol_type = genomic DNA
gttagagatattaaatctaggttaaaagtgagatcattactttgagagtgnagtctgtgatgagactgctcttgacattcccagctgcgatggacttcatg

SEQ ID NO: 79 - DNA, 101 bp, Brassica napus
  variation 51: note = n = A or G
  source 1..101: mol_type = genomic DNA
gagttaattaccttgagggtttcatgataccagaagcttctctaagaatantgagtttagtggtttttgcaaagtttaaaccaaataaacgaaggcctctc

SEQ ID NO: 80 - DNA, 101 bp, Brassica napus
  variation 51: note = n = C or G
  source 1..101: mol_type = genomic DNA
gagtgcaagtggaactgctacgccaccagtaaaccaagtcccaaggactcntgcaacaaactcagctgatggtttcgccacaattccaactctagctcctt

SEQ ID NO: 81 - DNA, 102 bp, Brassica napus
  variation 51: note = n = T or C
  variation 52: note = n = C or A
  source 1..102: mol_type = genomic DNA
ggataggttagaagtagaaatagcaacaaaaattcaaacagatcaccatanntacatttttagtatcttccttgtggaacaacttagagatggtaaaggcag

SEQ ID NO: 82 - DNA, 101 bp, Brassica napus
  variation 51: note = n = G or T
  source 1..101: mol_type = genomic DNA
ccacaaaccacctctcctttattcgacacggcaatgagtccagcgaacccntcgtcgagccgatgcttgatgacgtaatcaaccgcttcttgtagcccaac

SEQ ID NO: 83 - DNA, 101 bp, Brassica napus
  variation 51: note = n = C or A
  source 1..101: mol_type = genomic DNA
gcttctttggccagcttgagcatccccacgttgtcttccgtgacgaagtantcgttgtccacagtttcaactccctaataaaaaccaaatcgatttcggtt

SEQ ID NO: 84 - DNA, 101 bp, Brassica napus
  variation 51: note = n = T or C
  source 1..101: mol_type = genomic DNA
cagcaagtgaccaggaaggtcatgttccagtgactcacttgagtaaaagtnccagtacttatcagcaatacggttaactttctcaatgcattccaagcttg

SEQ ID NO: 85 - DNA, 101 bp, Brassica napus
  variation 51: note = n = C or T
  source 1..101: mol_type = genomic DNA
ttgtagatcgatcgttgcgtagagctgtgtttctcctttgccgaaaccaantgcctcttcaacatttgctctaatctggaatttaaaacagaggttaaaga

SEQ ID NO: 86 - DNA, 101 bp, Brassica napus
  variation 51: note = n = C or T
  source 1..101: mol_type = genomic DNA
tggtgctttcctttaggcctcacaacataagtccttccaaactcatatccncttgtacttctactacctacaaatattttgccagaaacatattcaagcag

SEQ ID NO: 87 - DNA, 101 bp, Brassica napus
  variation 51: note = n = T or G
  source 1..101: mol_type = genomic DNA
ggaagtcgagagatgatgagtgctcaacggttcctgagaatgagtctgtgnttggttcagaatgtcagtttgaatattctgatgatgatgatactgcacat

SEQ ID NO: 88 - DNA, 101 bp, Brassica napus
  variation 51: note = n = A or T
  source 1..101: mol_type = genomic DNA
gatgatgagtgctcaacggttcctgagaatgagtctgtgtttggttcagantgtcagtttgaatattctgatgatgatgatactgcacatggaagtgtgca

SEQ ID NO: 89 - DNA, 101 bp, Brassica napus
  variation 51: note = n = A or C
  source 1..101: mol_type = genomic DNA
caaggaaagcaaaagatgaaggtaaaggtgtgagggcaggagaagatggtnttgtgaatacatcagcaaactctaaagaagattctagaggtgttacacat

SEQ ID NO: 90 - DNA, 101 bp, Brassica napus
  variation 51: note = n = G or A
  source 1..101: mol_type = genomic DNA
cgatcctcgctacggaactctccacttccaaagctagtggaaggccgcaantatccccacgcacctgaggcatctccctcattatcttcttcctcgtcaaa

SEQ ID NO: 91 - DNA, 101 bp, Brassica napus
  variation 51: note = n = C or A
  source 1..101: mol_type = genomic DNA
agataaaaaccgaaccttttggaaggttctcaagctagacagaacagagcnaacagagctcaactttcaccaagactcaactccttacacagaatccgaat

SEQ ID NO: 92 - DNA, 103 bp, Brassica napus
  variation 51: note = n = C or absent
  variation 52: note = n = T or absent
  variation 53: note = n = T or absent
  source 1..103: mol_type = genomic DNA
cacattaaaccggaagctaatcgccggtgtcgacgacgagagctccgaagnnncttcttcttctactactcatggtgcttctttgaggattgatcgtgtttcg

SEQ ID NO: 93 - DNA, 101 bp, Brassica napus
  variation 51: note = n = G or A
  source 1..101: mol_type = genomic DNA
attatctattgatacctgagtcttaagccgctgatgactagtagaatgagnctcatcgaagcagaaaataaaacggtactgctaataaccaatccaagata

SEQ ID NO: 94 - DNA, 101 bp, Brassica napus
  variation 51: note = n = A or T
  source 1..101: mol_type = genomic DNA
tgatacctgagtcttaagccgctgatgactagtagaatgaggctcatcgangcagaaaataaaacggtactgctaataaccaatccaagatatatagagac

SEQ ID NO: 95 - DNA, 101 bp, Brassica napus
  variation 51: note = n = G or T
  source 1..101: mol_type = genomic DNA
aagccgctgatgactagtagaatgaggctcatcgaagcagaaaataaaacngtactgctaataaccaatccaagatatatagagaccacgcttggaactct

SEQ ID NO: 96 - DNA, 101 bp, Brassica napus
  variation 51: note = n = C or T
  source 1..101: mol_type = genomic DNA
cctctgtatttgacatctcttacatctataaatgtcccttatttttacttnactgtaagtttggacccaaatcttgtcttgttgaaaattctgccattgaa

SEQ ID NO: 97 - DNA, 101 bp, Brassica napus
  variation 51: note = n = T or C
  source 1..101: mol_type = genomic DNA
ggtttcggcctttcaaaaaaaactgttaaccaaatttcgaaatgaacgaancgaattaatcaaaattttgaactgaaattaccatagtttaattgaatttt

SEQ ID NO: 98 - DNA, 101 bp, Brassica napus
  variation 51: note = n = A or T
  source 1..101: mol_type = genomic DNA
cgcaaacgccaagtcaggagcattaatcgctgaaactccacttgttgttgngttggcttttgcagcagaaggtggcttagcagtagcggcagtagcagcag

SEQ ID NO: 99 - DNA, 101 bp, Brassica napus
  variation 51: note = n = A or T
  source 1..101: mol_type = genomic DNA
gaggtttctacacttatgatgcctttatcaccgccgctaaatatttccctngcttctgcaacaatggagacactgccgcaaggaagaaagagctctctgcc

SEQ ID NO: 100 - DNA, 101 bp, Brassica napus
  variation 51: note = n = A or T
  source 1..101: mol_type = genomic DNA
aatcgcaaatgtttcctgtcacagggacaagcttgtctaacatgaaagacntgaaagatgctccatgagtctctcttagatttttaaaaagctctgcatct

SEQ ID NO: 101 - DNA, 101 bp, Brassica napus
  variation 51: note = n = A or T
  source 1..101: mol_type = genomic DNA
cgcaaatgtttcctgtcacagggacaagcttgtctaacatgaaagacatgnaagatgctccatgagtctctcttagatttttaaaaagctctgcatctaac

SEQ ID NO: 102 - DNA, 101 bp, Brassica napus
  variation 51: note = n = G or A
  source 1..101: mol_type = genomic DNA
tatcttattaaaatagaaatacatttaaagaatatttggaaacataaatancagtaaaaaaatataatattatttgaaaacatagatatcaatatattaaa

SEQ ID NO: 103 - DNA, 101 bp, Brassica napus
  variation 51: note = n = C or T
  source 1..101: mol_type = genomic DNA
cttagacaaagactcagcggactctttgtgcacaatagtaacggcatcacngtcttcaggcacaggaagagacgaaatggtttcagattccctgggaggac

SEQ ID NO: 104 - DNA, 101 bp, Brassica napus
  variation 51: note = n = G or T
  source 1..101: mol_type = genomic DNA
ttcaagtccctaacctctctcaaagccataaaagctcttaactagagaatnaattcaaagaccaatagtatgttcttcattcccactatttatagcctatc

SEQ ID NO: 105 - DNA, 101 bp, Brassica napus
  variation 51: note = n = G or A
  source 1..101: mol_type = genomic DNA
gatcatgttccgttttggatacttatgaggaaatacaccaagttgaggcgnctagacgaggcgtatttagcttacaaaaaatggctaatgacgaggagcag

SEQ ID NO: 106 - DNA, 101 bp, Brassica napus
  variation 51: note = n = G or C
  source 1..101: mol_type = genomic DNA
gcgttgaaaaacttcggcgacaccaccaccgccggtaccgccgctccgtcntcctcctcctcgccgtcgttaacgaccgtgatcattggagccttgaagct

SEQ ID NO: 107 - DNA, 101 bp, Brassica napus
  variation 51: note = n = C or T
  source 1..101: mol_type = genomic DNA
ttaagctcaagaaagccaaagtcagacctgttctgttctcgtgtccgacgntccggtggggaaaccggcagccacggtcgagaaaagaagctgaatcagcg

SEQ ID NO: 108 - DNA, 101 bp, Brassica napus
  variation 51: note = n = A or C
  source 1..101: mol_type = genomic DNA
tatatataggtggatctctgagtgctgtggaaacattagcttctacttctntttgcggaatcatccacgccatctttggtggacagccattgttgatactt

SEQ ID NO: 109 - DNA, 101 bp, Brassica napus
  variation 51: note = n = T or C
  source 1..101: mol_type = genomic DNA
tgttttaataggacgagctatgggaagcctgtttaacaatgatgggcctgnctgcaaaccctgaaacgtctccccagaatttagcattgtacaaactttaa

SEQ ID NO: 110 - DNA, 101 bp, Brassica napus
  variation 51: note = n = G or A
  source 1..101: mol_type = genomic DNA
tccgaaatacccaaaaatacctaatctaaacaaaatatttcgcatactttnggtacccgatcgggtctccggtaagatccagacccaaaccgagatcgtat

SEQ ID NO: 111 - DNA, 101 bp, Brassica napus
  variation 51: note = n = C or T
  source 1..101: mol_type = genomic DNA
tacatgctaacagtgatgtgatattgttcactcgagggagttgaaacgggntctacggctgcacccagggcgaatctagttgttgtttggacaaaaccgga

SEQ ID NO: 112 - DNA, 101 bp, Brassica napus
  variation 51: note = n = C or T
  source 1..101: mol_type = genomic DNA
gatgcttccaccgacggcgttggcttactcagctccccccgtgacggcggnggtggcgttgatagtcttgattacgaggttatcgagaattacgcttacag

SEQ ID NO: 113 - DNA, 101 bp, Brassica napus
  variation 51: note = n = A or C
  source 1..101: mol_type = genomic DNA
ccgtgtggtggtagaatcatcgacgaggtttgcgtcaaggaggagtacganactcgtcccgggaagcgcttcttcagctgcataaactacgaggtaaccca

SEQ ID NO: 114 - DNA, 101 bp, Brassica napus
  variation 51: note = n = A or G
  source 1..101: mol_type = genomic DNA
tataaagccagcggtagcgcttacggtttatggcgataaatcagcgttctncaactgcggtttcttggggttacaagatacgttgtgggatgttcaaggca

SEQ ID NO: 115 - DNA, 101 bp, Brassica napus
  variation 51: note = n = C or T
  source 1..101: mol_type = genomic DNA
aggtcttctgccaaacccaagcctgtggatcaacgttgctccttggtacanaccaagtgagtgatgggctttcaccatctcctttactgcttcgataagtt

SEQ ID NO: 116 - DNA, 101 bp, Brassica napus
  variation 51: note = n = G or A
  source 1..101: mol_type = genomic DNA
caataatgtgaactctcttttcttcaagaagtctagcccaaacctgatcanagcctggttaagtagcacaccatctcctttaaggaaaaagcctcttcctc

SEQ ID NO: 117 - DNA, 101 bp, Brassica napus
  variation 51: note = n = G or A
  source 1..101: mol_type = genomic DNA
cgtcgatagttcacagagacaacaacgaaaccgcacagaccaacaagcctncggcaaagagtgtcatagatagcactgtttgcagaagagtgcacaaagct

SEQ ID NO: 118 - DNA, 101 bp, Brassica napus
  variation 51: note = n = C or T
  source 1..101: mol_type = genomic DNA
gttacagatacgagatggagccgtttggtgtacggttaaaggccttttgcntactctcttccgtggagaagaaggtgttcaacgagctgataccttcgctc

SEQ ID NO: 119 - DNA, 101 bp, Brassica napus
  variation 51: note = n = G or T
  source 1..101: mol_type = genomic DNA
taatcccttagattcaagcagcaacgcctgtaggctccctgagattcattncccattatgtgtcttagccatcaatctaattgcataaactagtaagaaca

SEQ ID NO: 120 - DNA, 101 bp, Brassica napus
  variation 51: note = n = A or G
  source 1..101: mol_type = genomic DNA
gccggtctagcacataagatctcaaacaaaacaactccaaaagagtacacntctgacttctcggttaactgctgtctcctaaagtactctggatctaagta

SEQ ID NO: 121 - DNA, 101 bp, Brassica napus
  variation 51: note = n = G or A
  source 1..101: mol_type = genomic DNA
tggttggactgtgcaatccgaggctttgactctttcatcaggatggggacngcatcgtttttctcaatcatgtggtgtggtgtcttctcagcgttttccat

SEQ ID NO: 122 - DNA, 101 bp, Brassica napus
  variation 51: note = n = C or T
  source 1..101: mol_type = genomic DNA
cttggataagagtttataggaaaaccaaaattcgcttgcaatgagtatcantattaaaccgatcatgaggagatcataacaactagaatttaaatgatgtt

SEQ ID NO: 123 - DNA, 101 bp, Brassica napus
  variation 51: note = n = C or T
  source 1..101: mol_type = genomic DNA
ttgtgtagatggtcgcatattgcaacacatctaccaggaagaacagacaangaaattaaaaactattggaattcacatctcagccgcaaaatctatgcctt

SEQ ID NO: 124 - DNA, 101 bp, Brassica napus
  variation 51: note = n = G or A
  source 1..101: mol_type = genomic DNA
acagggtatacctgtggctgagacattctataataaggctccttcaagtcnagtttgcacgatgatgtttggaggattccaccttgattggcaccgggccc

SEQ ID NO: 125 - DNA, 101 bp, Brassica napus
  variation 51: note = n = C or A
  source 1..101: mol_type = genomic DNA
ctccctactgatttctccaggtttaagacgatgcacatgtacgatatcgtngtcaagaaccgcaacatgttccaagtaagggatatgtagccatattttgg

SEQ ID NO: 126 - DNA, 101 bp, Brassica napus
  variation 51: note = n = C or A
  source 1..101: mol_type = genomic DNA
aaacgagatgattttcctttaaacgtttaaaaaaaatcaatctaggcattnaaaaaatcggtctgccaatactattagctattttctgaactttggttgct

SEQ ID NO: 127 - DNA, 101 bp, Brassica napus
  variation 51: note = n = G or A
  source 1..101: mol_type = genomic DNA
gcagcggttcgctccttcttgtgggcgttctggtggcctcctaaggcttgngaactctggaactttctagaacaaaagaggcaagagaatactcttgatga

SEQ ID NO: 128 - DNA, 101 bp, Brassica napus
  variation 51: note = n = G or A
  source 1..101: mol_type = genomic DNA
tttaggaagattagacagaacataccactgtcagtagctttgacgatggcnttactgataagaggcactttagaacacatggcagcaactacctgcatgct

SEQ ID NO: 129 - DNA, 101 bp, Brassica napus
  variation 51: note = n = G or A
  source 1..101: mol_type = genomic DNA
tccctctgtcaaaactgtgactggttaggccacaacggagctactaattcncatcataagaagcaaaccattaactgctactctggttgcccctcgagtga

SEQ ID NO: 130 - DNA, 101 bp, Brassica napus
  variation 51: note = n = G or A
  source 1..101: mol_type = genomic DNA
ctgtgataaacgatctttgaccatcattacaactatttcattagggaccanttgtcctttctccatgtgttcttttgccagtcttccattctcactcccag

SEQ ID NO: 131 - DNA, 101 bp, Brassica napus
  variation 51: note = n = G or A
  source 1..101: mol_type = genomic DNA
ggaactattttctaagtggatcaactcaaacgacgccgtttcgcttctatntaagtcaaaagtcctctcttttatgttttgtatccaaagtatacagcttt

SEQ ID NO: 132 - DNA, 101 bp, Brassica napus
  variation 51: note = n = A or G
  source 1..101: mol_type = genomic DNA
gagttttgcagcagcagattacttcccggtaattggtaaattaatcgatangatcacagggttacatagcaaatgtgagaaggtttttaaagcaatggatg

SEQ ID NO: 133 - DNA, 101 bp, Brassica napus
  variation 51: note = n = G or A
  source 1..101: mol_type = genomic DNA
tgagaaggtttttaaagcaatggatgcatttttcgatcaatctataaagcntcatcttgaagacgagagccttgaagatgatatcatagccttgctcttaa

SEQ ID NO: 134 - DNA, 101 bp, Brassica napus
  variation 51: note = n = A or C
  source 1..101: mol_type = genomic DNA
ttgatcttgaagaatcatatggactcgtatgtcctaagaaagttccacttnagcttatcccgattcttactcaatggacttgacttcgatttatattttcg

SEQ ID NO: 135 - DNA, 101 bp, Brassica napus
  variation 51: note = n = A or C
  source 1..101: mol_type = genomic DNA
actgaaacttcctataattggcaacttgcaccaattaggctcgcagcctcntcgttcattaacaaaattatctgaaaagtatggacctctaatgtccctaa

SEQ ID NO: 136 - DNA, 101 bp, Brassica napus
  variation 51: note = n = C or T
  source 1..101: mol_type = genomic DNA
gttgtatgaaagtcttgcagcgtacgtcaagtaaggtcgtgaacaacaatnaacgtcgaacgttttcaagacatctttcactgtctctggagttgacgcca

SEQ ID NO: 137 - DNA, 101 bp, Brassica napus
  variation 51: note = n = A or C
  source 1..101: mol_type = genomic DNA
ttcaagacatctttcactgtctctggagttgacgccacaacggtagacacntttccaagcttgagggacattagagctccatatttttcagataatttgaa

SEQ ID NO: 138 - DNA, 101 bp, Brassica napus
  variation 51: note = n = G or T
  source 1..101: mol_type = genomic DNA
catggaacgatgaggtttggatcccagttggtgcaagttaccaattataanaagccttggtggtccaggaggtaggtttttctttgtctttctcgtgttct

SEQ ID NO: 139 - DNA, 101 bp, Brassica napus
  variation 51: note = n = A or C
  source 1..101: mol_type = genomic DNA
ctacatatgcatagtttccatacagagagatagatagcagtaggtataagntaagctaagctaacctacagatataatgaaacaattacaagatgtaaaaa

SEQ ID NO: 140 - DNA, 101 bp, Brassica napus
  variation 51: note = n = G or C
  source 1..101: mol_type = genomic DNA
tttagctgtgttcctcacctcattcgccaattctatcacgaactcttcctnatgctctgttttgacaacattcatcaaacaaatcaacgccaaaaatctta

SEQ ID NO: 141 - DNA, 101 bp, Brassica napus
  variation 51: note = n = T or C
  source 1..101: mol_type = genomic DNA
ttgaaaaggtactcccggttgttcccacaaggttcagaagctgtggcgatntgcatggccatctcctccaaaagtagtatttgttcgacactttgtctggt

SEQ ID NO: 142 - DNA, 101 bp, Brassica napus
  variation 51: note = n = T or C
  source 1..101: mol_type = genomic DNA
tcgacaagggttttagagtcgtattcacactctcttcgttccaagtactcnatggctagcttctccttctcaggtcctccatgaacacaataagcagcacc

SEQ ID NO: 143 - DNA, 101 bp, Brassica napus
  variation 51: note = n = A or G
  source 1..101: mol_type = genomic DNA
caacattgatctatttctttgttgataattcaagaacaagtgtgttgtaancataccgagatcaaagacgcgtttgtagtctttaatattgccaatgagtt

SEQ ID NO: 144 - DNA, 101 bp, Brassica napus
  variation 51: note = n = T or A
  source 1..101: mol_type = genomic DNA
gtgtgttgtaaacataccgagatcaaagacgcgtttgtagtctttaatatngccaatgagtttttcgtcaaaatcgaaacctgggttccatattatagatc

SEQ ID NO: 145 - DNA, 101 bp, Brassica napus
  variation 51: note = n = G or T
  source 1..101: mol_type = genomic DNA
ccgagatcaaagacgcgtttgtagtctttaatattgccaatgagtttttcntcaaaatcgaaacctgggttccatattatagatccatatccgaattccca

SEQ ID NO: 146 - DNA, 102 bp, Brassica napus
  variation 51: note = n = T or C
  variation 52: note = n = T or A
  source 1..102: mol_type = genomic DNA
ttttcgtcaaaatcgaaacctgggttccatattatagatccatatccgaanncccacaataccatcttgaacgttaaagtctcttttaggaagaagaaatag

SEQ ID NO: 147 - DNA, 101 bp, Brassica napus
  variation 51: note = n = A or T
  source 1..101: mol_type = genomic DNA
cctaaatcaagcaagcttctcgattcgattcgactcgatcatcatcatgcnctcgaatcatctacagaacgggatcgaatccgataatctcctctggtctc

SEQ ID NO: 148 - DNA, 102 bp, Brassica napus
  variation 51: note = n = G or A
  source 1..102: mol_type = genomic DNA
acttaattcactttttaaaactgttactattatcacacactaccatttaanattgccttcttctttgagttataggcaagttggaaaagccttctttttgta

SEQ ID NO: 149 - DNA, 101 bp, Brassica napus
  variation 51: note = n = A or G
  source 1..101: mol_type = genomic DNA
aagggtaacgattctccggcgcacgtcgatagttcacagagacaacaacgnaaccgcacagaccaacaagcctgcggcaaagagtgtcatagatagcactg

SEQ ID NO: 150 - DNA, 101 bp, Brassica napus
  variation 51: note = n = T or A
  source 1..101: mol_type = genomic DNA
gattctagcattttactcacagtgagaacatcggcaagcgtagtcttttcntcatcggagttagctcgagcgttaagagtggcagctgactgagcagaagc

SEQ ID NO: 151 - DNA, 101 bp, Brassica napus
  variation 51: note = n = G or A
  source 1..101: mol_type = genomic DNA
ttctgcctcttgagagaaaatcatgccaacgggtaggttcgagacgatgcntgaatgggttcacgacgccatctctgctcaacgcaatgagctcctctctc

SEQ ID NO: 152 - DNA, 101 bp, Brassica napus
  variation 51: note = n = A or C
  source 1..101: mol_type = genomic DNA
tatagtctctccaggagctgatatgaccatatactttccttattctgacanggaaagaagactaactgcccttcatgagtctattgaagaacttctgttta

SEQ ID NO: 153 - DNA, 101 bp, Brassica napus
  variation 51: note = n = A or G
  source 1..101: mol_type = genomic DNA
atgataatttttttctggttttgcaggccaattcaatcccactggcaactnatgagcattgagcaagctatggttggattctaatacttgctgcactccct

SEQ ID NO: 154 - DNA, 103 bp, Brassica napus
  variation 51: note = n = C or T
  variation 53: note = n = C or T
  source 1..103: mol_type = genomic DNA
tacaaatttggctcaagtctgggcacaaacaacagctagttttccatagcngnatcaggaaggggagaatcatgagccagtgactgcagaagagagatacaac

SEQ ID NO: 155 - DNA, 101 bp, Brassica napus
  variation 51: note = n = T or C
  source 1..101: mol_type = genomic DNA
atgaaatttaggtcctgtgaagcaaatgaccttgtacagtcctttgtaatnaagagtgccaagatctgctgaaatagatccatcatccaatctatcaacca

SEQ ID NO: 156 - DNA, 101 bp, Brassica napus
  variation 51: note = n = G or C
  source 1..101: mol_type = genomic DNA
tgaatggtgttgaaaagtttaccatacctgacagctttatttgataacatnttcgtaccgtgcatggctctctgcacatacccaaatctatcagccttgac

SEQ ID NO: 157 - DNA, 101 bp, Brassica napus
  variation 51: note = n = A or G
  source 1..101: mol_type = genomic DNA
taccggagaagaataagcggtgaacctcagcttcttcgccatatctccacncataagcattctgtcgaacacctgatgggcgcatagattcatttttcact

SEQ ID NO: 158 - DNA, 101 bp, Brassica napus
  variation 51: note = n = T or G
  source 1..101: mol_type = genomic DNA
cggagaagaataagcggtgaacctcagcttcttcgccatatctccacacanaagcattctgtcgaacacctgatgggcgcatagattcatttttcactttt

SEQ ID NO: 159 - DNA, 101 bp, Brassica napus
  variation 51: note = n = A or C
  source 1..101: mol_type = genomic DNA
aatgttgtggcaggcgttgtctataagtaggtcactggccatggctaaagnttctgcacaacaagggcaaaccagttgtaaagagcttaaggatatgataa

SEQ ID NO: 160 - DNA, 101 bp, Brassica napus
  variation 51: note = n = C or G
  source 1..101: mol_type = genomic DNA
ggcaggcgttgtctataagtaggtcactggccatggctaaagattctgcanaacaagggcaaaccagttgtaaagagcttaaggatatgataatttcagag

SEQ ID NO: 161 - DNA, 101 bp, Brassica napus
  variation 51: note = n = A or G
  source 1..101: mol_type = genomic DNA
ggtcactggccatggctaaagattctgcacaacaagggcaaaccagttgtnaagagcttaaggatatgataatttcagagattgttggagctgcaggaaga

SEQ ID NO: 162 - DNA, 101 bp, Brassica napus
  variation 51: note = n = C or G
  source 1..101: mol_type = genomic DNA
gtgtgtacaacatgcagattgttgatcaagaaactcttgaaggagtagaanagatagagagtggagtagtgatttgtgttgctgcctggtttggaacggtt

SEQ ID NO: 163 - DNA, 103 bp, Brassica napus
  variation 51: note = n = G or A
  variation 53: note = n = A or T
  source 1..103: mol_type = genomic DNA
ctgcctggtttggaacggttaggctcatagacaacattgagatcaatgtcncngtctaattcccactttccgcatccatctctggttcgttgacaggagcttc

SEQ ID NO: 164 - DNA, 101 bp, Brassica napus
  variation 51: note = n = C or G
  source 1..101: mol_type = genomic DNA
aaaatccgagctcttgttctggttagactgggtctggtttgaaggaggagnaacaaacttggttttggctgggacagtggcggagtcaatatcaccgacgt

SEQ ID NO: 165 - DNA, 101 bp, Brassica napus
  variation 51: note = n = C or T
  source 1..101: mol_type = genomic DNA
ctggttagactgggtctggtttgaaggaggagcaacaaacttggttttggntgggacagtggcggagtcaatatcaccgacgtagtactcatcaagcatgg

SEQ ID NO: 166 - DNA, 101 bp, Brassica napus
  variation 51: note = n = T or G
  source 1..101: mol_type = genomic DNA
cgtcgatgacgatccaacagtcctggttgctagtgtgctgagaaacctccnccaaggtgaacactttgccgtctccgcccatctcccgatgatcctcaatt

SEQ ID NO: 167 - DNA, 101 bp, Brassica napus
  variation 51: note = n = G or C
  source 1..101: mol_type = genomic DNA
agcgttcggattctttacgaacatcaaaagcttatactccggacaagttcnagtcaaaatctcacgtagaataatctcaacggtttcactaaatcttctag

SEQ ID NO: 168 - DNA, 101 bp, Brassica napus
  variation 51: note = n = T or G
  source 1..101: mol_type = genomic DNA
ggaacgcggtttcctgcgtttccgcctctggttttcaattttaccgcggangatcaaccgttgattttgcagacttcgagacttgctacggaagtaaagat

SEQ ID NO: 169 - DNA, 101 bp, Brassica napus
  variation 51: note = n = C or T
  source 1..101: mol_type = genomic DNA
ctctggttttcaattttaccgcggatgatcaaccgttgattttgcagactncgagacttgctacggaagtaaagatgattaagtacggagaagcggttgaa

SEQ ID NO: 170 - DNA, 101 bp, Brassica napus
  variation 51: note = n = C or T
  source 1..101: mol_type = genomic DNA
gagacttgctacggaagtaaagatgattaagtacggagaagcggttgaaanggtttttcaagggacgagtttaggtggtggtggaatcgaccaccccatgc

SEQ ID NO: 171 - DNA, 101 bp, Brassica napus
  variation 51: note = n = G or A
  source 1..101: mol_type = genomic DNA
actatgatctcagaagttttggacccatatttgaagccatccaagatgtgnttgcatcgacgagctgggcaagatctttggacgtgtatgcacgaagaatc

SEQ ID NO: 172 - DNA, 103 bp, Brassica napus
  variation 51: note = n = G or A
  variation 53: note = n = C or T
  source 1..103: mol_type = genomic DNA
tttggacccatatttgaagccatccaagatgtggttgcatcgacgagctgngnaagatctttggacgtgtatgcacgaagaatcttcgagtccattccaagcg

SEQ ID NO: 173 - DNA, 101 bp, Brassica napus
  variation 51: note = n = G or A
  source 1..101: mol_type = genomic DNA
aagccatccaagatgtggttgcatcgacgagctgggcaagatctttggacntgtatgcacgaagaatcttcgagtccattccaagcgtacctccaacctct

SEQ ID NO: 174 - DNA, 101 bp, Brassica napus
  variation 51: note = n = A or C
  source 1..101: mol_type = genomic DNA
catccaagatgtggttgcatcgacgagctgggcaagatctttggacgtgtntgcacgaagaatcttcgagtccattccaagcgtacctccaacctctttag

SEQ ID NO: 175 - DNA, 101 bp, Brassica napus
  variation 51: note = n = G or C
  source 1..101: mol_type = genomic DNA
actccatccaatacagctaagctactgcgaaagaaaatcaatcacctcganttctgaccagtcggcggatccagcggagctactttgagattgcttgacag

SEQ ID NO: 176 - DNA, 101 bp, Brassica napus
  variation 51: note = n = A or G
  source 1..101: mol_type = genomic DNA
gactcttcgtgagacgcgctctttgtcgaatctgatcaccgagaatggagnccggcgagtggaagagaagttagagagtctccggcggggagagaaagacg

SEQ ID NO: 177 - DNA, 101 bp, Brassica napus
  variation 51: note = n = C or A
  source 1..101: mol_type = genomic DNA
tctaggttgcagcccaaatccggtagcctcggatcttttcataatcctcancctcctgcatgactctatgaacattctgcaaaatcaaaatacccaaccca